(19) United States
(12) Patent Application Publication
(10) Pub. No.: US 2026/0345699 A1
(43) Pub. Date: Nov. 5, 2026

(54) MICROBIOME INGREDIENT COMBINATION FOR LOWERING UREMIC TOXINS IN CARDIOMETABOLIC OR NEURODEGENERATIVE CONDITIONS

(71) Applicant: SOCIÉTÉ DES PRODUITS NESTLÉ S.A., Vevey (CH)

(72) Inventors: Sophie Hélène NUTTEN, Palézieux-Village (CH); Stéphane DUBOUX, St-Prex (CH); Amaury PATIN, Lausanne 26 (CH); Lisa Marcela LAMOTHE, Lausanne 26 (CH); Claudia ROESSLE, Morges (CH); Maureen HUHMANN, Branchburg, NJ (US); Jane Mea M NATÎVIDAD, Lausanne (CH); Maximilian VON EYNATTEN, La Conversion (CH)

(21) Appl. No.: 19/148,591

(22) PCT Filed: Jan. 16, 2024
(86) PCT No.: PCT/EP2024/050839

§ 371 Date: Jul. 16, 2025

Related U.S. Application Data
(60) Provisional application No. 63/439,638, filed on Jan. 18, 2023.
(60) Provisional application No. 63/480,729, filed on Jan. 20, 2023.

(51) Int. Cl. A61K 35/747; A61K 31/20; A61K 31/7016; A61K 31/702; A61P 13/12; C12N 1/20; C12N 9/26; C12N 9/34; C12N 9/42; C12R 1/225

(52) U.S. Cl. CPC A61K 35/747; A61K 31/20; A61K 31/7016; A61K 31/702; A61P 13/12; C12N 1/20; C12N 9/2428; C12N 9/2431; C12N 9/2437; C12N 9/2445; C12N 9/2448; C12R 2001/225; C12Y 302/01003; C12Y 302/01004; C12Y 302/01022; C12Y 302/01023; C12Y 302/01026; C12Y 302/01073

(57) ABSTRACT

The invention relates to composition for use in lowering or avoiding accumulation of uremic toxins in cardiometabolic or neurodegenerative conditions, particularly compositions that comprise a probiotic bacteria, a prebiotic (carbohydrates or fibers), and a lipid.

### CROSS REFERENCE TO RELATED APPLICATIONS

The present application is a National Stage of International Application No. PCT/EP2024/050839 filed Jan. 16, 2024, which claims priority to U.S. Patent Application No. 63/480,729 filed Jan. 20, 2023, and U.S. Patent Application No. 63/439,638 filed Jan. 18, 2023, the entire contents of which are incorporated herein by reference.

### SEQUENCE LISTING

The instant application contains a Sequence Listing which has been submitted electronically in XML file format and is hereby incorporated by reference in its entirety. Said XML copy, created on Jul. 16, 2025, is named 3712036-05065_SL.xml and is 1,994,647 bytes in size.

### BACKGROUND OF THE INVENTION

The present disclosure generally relates to compositions and methods that allow lowering or avoiding accumulation of uremic toxins, preferably in cardiometabolic or neurodegenerative conditions, more preferably in chronic kidney disease (CKD). Such a composition comprises a specifically selected prebiotic, a specifically selected probiotic and a specifically selected lipid as defined herein.

Uremic toxins typically can be classified into three categories: free water-soluble low-molecular-weight solutes, protein-bound solutes, and middle molecules, which are usually products of protein/amino acid metabolism by body tissues, particularly the liver. Uremic toxins include among others for example urea, indoxyl sulfate, TMAO, p-cresol sulfate, 3-carboxy-4-methyl-5-propyl-2-furanpropionic acid, p-cresylglucuronide and uric acid. An accumulation of such systemic uremic toxins, also known as uremia or uremic state, is a condition often observed in individuals with cardiometabolic and neurological conditions, including but not limited to chronic kidney disease (CKD).

Chronic kidney disease for example, is a disease that is characterized by a progressive and gradual loss of renal function and may lead to an accumulation of such uremic toxins, normally cleared by the kidneys, resulting in uremia. CKD patients have increased risk of developing cardiovascular disease (CVD), due to an assortment of CKD-specific risk factors. The accumulation of uremic toxins in the circulation and in tissues is associated with the progression of CKD and its co-morbidities, including CVD (see Yong Jin Lim et al., Toxins 2021, 13, 142).

Chronic kidney disease (CKD) and CVD are not the sole conditions that show elevated levels of uremic toxins but also concern other cardiometabolic and neurological conditions. Moreover, early indicators of excessive uremic toxin contents are often widespread and not always clearly attributable to such cardiometabolic and neurological conditions as a primary cause at first sight. Generally, the accumulation of uremic toxins may lead to syndromes, and symptom manifestations, including but not exhaustive to fatigue, anorexia and protein energy wasting, which ultimately affect the quality of life of individuals afflicted with high levels of circulating uremic toxins. In this context, several publications support the causal role of uremic toxins, including indoxyl sulfate, p-cresyl sulfate, TMAO and urea, in inducing tissue damages and impairments such as the kidneys or other organs, leading to disease progression and organ failure and mortality [Reference: Vanholder R., Schepers E, Pletinck A, Nagler E V, Glorieux G. 2014. J Am Soc Nephrol. 25(9):1897-907. doi: 10.1681/ASN.2013101062; Falconi et al. 2021. Front Physiol. 12: 686249].

Therefore, poor management of systemic uremic toxins could lead to progression of disease, appearance of associated symptoms and syndromes (e.g. uremic syndrome, anorexia) and poor quality of life and even death of patients. Early intervention is therefore of high importance and essential in effectively treating such diseases to positively influence their outcome.

As generally indicated above, there are usually two main reasons contributing to disturbed levels of uremic toxins in the circulation. The first reason is typically an increased accumulation due to poor kidney filtration. The second reason is an altered production due to several modifications in metabolism including the liver, gut and microbiome.

In the prior art there are currently several proposals for managing uremic toxins and such causes for disturbed levels of uremic toxins.

Since some uremic toxins may stem also from a high protein content in the diet, one current effective solution to manage uremic toxins is to administer patients a low protein diet. A low protein diet is however often not sustainable as the patients usually require a balanced diet and good nutrition. For instance, protein is required to manage other complications associated with kidney disease (e.g. protein energy wasting). In many cases, a low protein diet is even detrimental, e.g., in case of anorexia and muscle dystrophy, as well as generally in elderly people, all of which require a minimum protein content if not even elevated protein levels to prevent or treat excessive muscle protein degradation (muscle catabolism).

A further method of addressing uremic toxins, which could be regarded as the gold standard in the management of kidney disease, is the removal of uremic toxins via dialysis. However, this requires continued treatment, that needs to be carried out in many cases in specific nursing facilities or hospitals, and is therefore burdensome and cost-intensive. Moreover, in late-stage diseases such as end-stage renal disease, uremic toxins cannot be removed by dialysis. In particular, dialysis is inefficient in removing protein-bound uremic toxins such as p-cresyl sulfate, indoxyl sulfate, 3-carboxy-4-methyl-5-propyl-2-furanpropionic acid and p-cresylglucuronide

There is also the possibility of reducing gut microbiota-derived metabolites, which are used as substrates for tissue uremic toxin metabolism. Such methods include e.g. (1) AST-120, which is a charcoal base solution that aims to remove the microbiota-derived precursors in the gut level; (2) the use of probiotics/live therapeutics to correct dysbiosis, to break down uremic toxin precursors; and/or (3) the use of fiber/glycans/oligosaccharides to correct dysbiosis.

However, the one drawback of such solutions is that they only partly address the production problem of uremic toxins and still may lead to or do not avoid an accumulation of uremic toxins in the body and the organs since the primary causes are not removed. There are also reports that uremic toxin accumulation in the systemic circulation may be at least partially independent from the production of uremic toxin precursors by the gut microbiota.

The prior art has also developed medicines, such as SGLT2 inhibitors intended to help improving glomerular hemodynamic function. Such SGLT2 inhibitors are thought to ameliorate other local and systemic mechanisms involved in the pathogenesis of CKD. However, these medicines have not been fully shown to improve uremic toxin levels in circulation and to provide a reliable basis for treatment of conditions caused by such uremic toxins, e.g. in individuals with cardiometabolic and neurological conditions.

Therefore, there exists a need in the art to provide an alternative and preferably improved concept of therapy to more effectively address such issues and to avoid and/or treat the accumulation of uremic toxins in a patient, preferably in individuals with cardiometabolic and neurological conditions, particularly chronic kidney disease.

The invention as described herein addresses such needs and provides compositions, uses and corresponding therapeutic methods, that avoid and/or treat the accumulation of uremic toxins in such patients. The invention also provides kits of parts for such purposes.

### SUMMARY OF THE INVENTION

The problems described above are solved by the subject matter of the independent claims and are described in further detail in the following specification, its embodiments and aspects, and the dependent claims. Embodiments and aspects as disclosed herein may be combined with each other as required and if not explicitly stated otherwise.

The invention is particularly directed to a new composition targeting multiple direct and indirect mechanisms that contribute to the avoidance of production of uremic toxins and prevent or at least lower the levels of accumulation of uremic toxins.

According to a first embodiment, there is a composition, preferably suitable for use in lowering and/or avoiding accumulation of uremic toxins in cardiometabolic or neurodegenerative conditions. The composition comprises a specifically selected probiotic bacterium, a specifically selected prebiotic and a specifically selected lipid. More precisely, the composition comprises:
(a) a probiotic bacterium selected from:
   a probiotic bacterium lacking a gene for producing at least one of urea, uric acid, p-cresol, p-cresyl sulfate, indoxyl sulfate, indole, indole acetic acid, indoxyl, trimethylamine, TMAO, dimethylglycine, betaine and/or glutarate; and/or
   a probiotic bacterium lacking at least one bacterial enzyme selected from urease, carnitine monooxygenase & reductase, tryptophanase and/or hydroxyphenylacetate; and/or
   a probiotic bacterium expressing at least one bacterial enzyme selected from of α-galactosidase, β-galactosidase, glucan 1,4-β-glucosidase, cellulase, β-fructofuranosidase and/or Licheninase;
(b) a prebiotic selected from carbohydrates, wherein the carbohydrate can be hydrolyzed by at least one bacterial enzyme selected from α-galactosidase, β-galactosidase, glucan 1,4-β-glucosidase, cellulase, β-fructofuranosidase and/or Licheninase;
(c) a lipid, selected from a triglyceride, a short chain fatty acid and/or a medium chain fatty acid, wherein the triglyceride comprises butyrate, short chain fatty acids, medium chain fatty acids, long chain fatty acids, or a mixture thereof.

According to a first aspect, the composition as defined herein comprises a probiotic bacterium. According to the first criterion, such a probiotic bacterium may be selected from probiotic bacteria, lacking a gene for producing at least one of urea, uric acid, p-cresol, p-cresyl sulfate, indoxyl sulfate, indole, indole acetic acid, indoxyl, trimethylamine, TMAO, dimethylglycine, betaine and/or glutarate. According to the second criterion, such a probiotic bacterium may also be selected from probiotic bacteria, lacking, at least one bacterial enzyme selected from urease, carnitine monooxygenase & reductase, tryptophanase and/or hydroxyphenylacetate. According to the third criterion, such a probiotic bacterium may be selected from probiotic bacteria, expressing at least one bacterial enzyme selected from α-galactosidase, β-galactosidase, glucan 1,4-β-glucosidase, cellulase, β-fructofuranosidase and/or Licheninase. Any of these three criteria may be applied alone or in combination with the other two criteria. Preferably, all three criteria are met.

Such a probiotic bacterium preferably meeting all three criteria may be selected from any of the following species or subspecies: Bifidobacterium animalis subspecies lactis, Bifidobacterium longum subspecies infantis, Bifidobacterium longum subspecies longum, Enterococcus faecium, Lactobacillus johnsonii, Lactococcus lactis, Lacticaseibacillus paracasei (previously classified as Lactobacillus paracasei), Limosilactobacillus reuteri (previously classified as Lactobacillus reuteri), Lacticaseibacillus rhamnosus (previously classified as Lactobacillus rhamnosus), Staphylococcus carnosus and/or Streptococcus thermophiles, preferably, Lactobacillus johnsonii, or a probiotic bacterium having a genome that has at least 95% Average Nucleotide Identity (ANI), preferably at least 96% ANI, more preferably at least 97% ANI, even more preferably at least 98% ANI, most preferably at least 99% ANI or even 99.5% ANI or 99.9% ANI to the genomic sequence of any one of the probiotic bacteria as defined before.

Even more preferably, such a probiotic bacterium preferably meeting all three criteria may be selected from any of the following strains:
a. Bifidobacterium animalis subspecies lactis NCC 2818, which was deposited on 7 Jun. 2005 with CNCM [Collection Nationale de Cultures de Microorganismes at Institute Pasteur, Paris, France] and assigned accession number CNCM I-3446);
b. Bifidobacterium longum subspecies infantis NCC 341 (ATCC 15697 (T)), which is available from worldwideweb.atcc.org/products/15697]);
c. Bifidobacterium longum subspecies longum NCC 2705 (CNCM I-2618) (NCBI refseq; GCA_000007525.1) [NCC 2075 was deposited on 29 Jan. 2001 with CNCM and assigned accession number CNCM I-2618];
d. Enterococcus faecium NCC 2768 (NCIMB 10415—which is available from Cerbios-Pharma SA Barbengo Switzerland (cerbios.swiss/e-faecium-sf68-a-model-for-efficacy-safety-for-pharmaceutical-probiotics/);
e. Lactobacillus johnsonii NCC 533 [originally known as La 1 which was deposited on 30 Jun. 1992 with CNCM and assigned accession number CNCM I-1225) (see also NCBI refseq; GCA_000008065.1, and GenBank AE017198.1 (included as SEQ ID NO: 1))]; f. Lactococcus lactis NCC 2287 (CNCM I-4154) [NCC 2287 was deposited on 24 Apr. 2009 with CNCM and assigned accession number CNCM I-4154];
g. Lacticaseibacillus paracasei NCC 2461 (CNCM I-2116) [NCC 2461 was deposited on 12 Jan. 1999 with CNCM and assigned accession number CNCM I-2116];
h. Lacticaseibacillus rhamnosus NCC 4007 (CGMCC 1.3724) [NCC 4007 was deposited in October 2004 with identification number CGMCC 1.3724 with CGMCC [China General Microbiological Culture Collection Center (CGMCC) Institute of Microbiology, Chinese Academy of Sciences, P.O. Box 2714, Beijing 100080, China];
i. Staphylococcus carnosus NCC 1052 (CNCM I-5400) [NCC 1052 was deposited on 1 Feb. 2019 with CNCM and assigned accession number CNCM I-5400];
j. Staphylococcus carnosus NCC 971 (CNCM I-5398) [NCC 971 was deposited with CNCM on 1 Feb. 2019 and assigned accession number CNCM I-5398]; and/or
k. Streptococcus thermophilus NCC 2496 (CNCM I-3915) [NCC 2496 was deposited with CNCM on 5 Feb. 2008 and assigned accession number CNCM I-3915];
   or a probiotic bacterium having a genome that has at least 95% Average Nucleotide Identity (ANI), preferably at least 96% ANI, more preferably at least 97% ANI, even more preferably at least 98% ANI, most preferably at least 99% ANI or even 99.5% ANI or 99.9% ANI to the respective genomic sequences according to any one of a. to k. as defined above. CNCM identifications refer to the Collection Nationale de Cultures de Microorganismes at Institut Pasteur, 22 rue du docteur Roux, 75724 Paris, France. CGMCC identifications refer to China General Microbiological Culture Collection Center (CGMCC) Institute of Microbiology, Chinese Academy of Sciences, P.O. Box 2714, Beijing 100080, China.

Even more preferably, the probiotic bacteria of the composition as defined herein is selected from Lactobacillus johnsonii NCC 533 (CNCM I-1225) or a probiotic having a genome that has at least 95% ANI, preferably at least 96% ANI, more preferably at least 97% ANI, even more preferably at least 98% ANI, most preferably at least 99% ANI or even 99.5% ANI or 99.9% ANI to the genomic sequence of Lactobacillus johnsonii NCC 533 (CNCM I-1225). The NCBI reference sequence of Lactobacillus johnsonii NCC 533 (CNCM I-1225) is GCA_000008065.1 (SEQ ID NO: 1).

According to a second aspect the composition as defined herein also comprises a prebiotic. Such a prebiotic is selected from carbohydrates, wherein the carbohydrate can be hydrolyzed by at least one bacterial enzyme selected from α-galactosidase, β-galactosidase, glucan 1,4-β-glucosidase, cellulase, β-fructofuranosidase and/or Licheninase. Such a carbohydrate is preferably a prebiotic selected from α-galacto-oligosaccharides, raffinose, β-galacto-oligosaccharides, and cello-oligosaccharides, or a combination thereof. Even more preferably, such a prebiotic is selected from pea galacto-oligosaccharides (α-GOS), soy galacto-oligosaccharides (α-GOS), β-galacto-oligosaccharides (β-GOS), bovine milk oligosaccharides (BMOS) (β-GOS), Vivinal GOS (β-GOS), cellobiose, cellotriose, cellotetraose, human milk oligosaccharides (HMOs), soluble hydrolyzed wheat, soluble hydrolyzed oat and/or β-glucan hydrolysates containing cellobiose or cellotriose or cellotetraose, or a combination thereof. Preferred HMOs include 2′-fucosyllactose (2′FL), 3-fucosyllactose (3′FL), lacto-N-neotetraose (LNnT), lacto-N-tetraose (LNT), 3′-sialyllactose (3SL), and/or 6′-sialyllactose (6SL), or a combination thereof. Particularly preferred HMOs include LNT, 2′FL, and/or LNnT, or a combination thereof.

According to a third aspect, the composition as defined herein also comprises a lipid (c). Such a lipid (c) is selected from a triglyceride, a short chain fatty acid and/or a medium chain fatty acid, wherein the triglyceride comprises short chain fatty acids, medium chain fatty acids, long chain fatty acids, or a mixture thereof. Preferred triglycerides include a triglyceride comprising butyrate, a triglyceride comprising butyrate and caprylate and a triglyceride comprising butyrate and oleate. More preferably, such a lipid (c) is selected from a. Triglycerides (TG) composed of a mixture of butyrate and medium chain fatty acids (MCFAs); b. Triglycerides (TG) composed of a mixture of short and medium chain fatty acids (SMCFAs); c. Triglycerides (TG) composed of a mixture of butyrate and long chain fatty acids (such as a mixture of butyrate and oleate); d. Triglycerides composed of a medium chain fatty acid; e. Triglycerides (TG) composed of a short chain fatty acid; f. Short chain fatty acids that could be metabolized into ketone bodies; and/or g Short and/or medium chain fatty acids that could be metabolized into ketone bodies, preferably C4 and/or C8 fatty acids (such as butyric acid (C4:0) and/or caprylic acid (C8:0)). Any combination is possible, e.g. a., b., c., d., a./b., a./c., a./d., a./e., a./f., a./g., b./c., b./d., b./e., b./f., c./d., c./e., c./f., a./b./c., a./d./c.; a./e./c., a./f./c., b./c./d., a./b./c./d., etc. In one embodiment, the composition defined herein comprises a triglyceride consisting of butyrate and caprylate. In one embodiment, the composition defined herein comprises a triglyceride consisting of butyrate and oleate.

According to a further aspect the probiotics are preferably contained in the composition as defined herein in an effective amount, preferably in an amount of between 10³ cfu to 10¹² cfu, typically in an amount of between 10⁴ cfu to 10¹¹ cfu per daily dose, preferably in an amount of between 10⁵ cfu to 10¹⁰ cfu per daily dose, or 10⁵ cfu to 10⁹ cfu per daily dose, likewise preferably in an amount of between 10⁶ cfu to 10⁹ cfu per daily dose, 10⁶ cfu to 10⁸ cfu per daily dose or in an amount of 10⁸ cfu to 10¹⁰ cfu per daily dose, more preferably around 10⁷ cfu to 10⁹ cfu per daily dose. A preferred daily dose is around 10⁸ total cfu per daily dose, 10⁷ to 10⁹ cfu per daily dose or 10⁸ to 10⁹ cfu per daily dose.

Moreover, according to another aspect, the prebiotics are preferably contained in the composition as defined herein in an amount of between 0.1 g to 30 g per daily dose, preferably in an amount of between 2 g to 15 g per daily dose.

According to a further aspect, the lipids are preferably contained in the composition as defined herein in an amount of between 0.1 to 30 g per daily dose, preferably in an amount of between 2 g to 15 g per daily dose.

According to a preferred aspect, the composition as defined herein may comprise a probiotic, a prebiotic and a lipid as defined above, wherein:
(a) the probiotic bacterium is selected from at least one of the probiotic bacteria according to a. to k. as defined above, or a probiotic having a genome that has at least 95% ANI, preferably at least 96% ANI, more preferably at least 97% ANI, even more preferably at least 98% ANI, most preferably at least 99% ANI or even 99.5% ANI or 99.9% ANI to any one of the genomic sequences of the probiotic bacteria according to a. to k. as defined above, or a combination thereof, more preferably selected from Lactobacillus johnsonii NCC 533 (CNCM I-1225) or a probiotic having a genome that has at least 95% ANI, preferably at least 96% ANI, more preferably at least 97% ANI, even more preferably at least 98% ANI, most preferably at least 99% ANI or even 99.5% ANI or 99.9% ANI to the genomic sequence of Lactobacillus johnsonii NCC 533 (CNCM I-1225) (SEQ ID NO: 1).
(b) the prebiotic is selected from α-galacto-oligosaccharides, raffinose, β-galacto-oligosaccharides, and cello-oligosaccharides, or a combination thereof, preferably selected from pea galacto-oligosaccharides (pea GOS, α-GOS), soy galacto-oligosaccharides (soy GOS, α-GOS), β-galacto-oligosaccharides (β-GOS), bovine milk oligosaccharides (e.g. Vivinal GOS, β-GOS), cellobiose, cellotriose, cellotetraose, soluble hydrolyzed wheat, soluble hydrolyzed oat, human milk oligosaccharides (HMOs), β-glucan hydrolysates containing cellobiose or cellotriose or cellotetraose, or a combination thereof;
(c) the lipid is selected from of a triglyceride, a short chain fatty acid and/or a medium chain fatty acid, wherein the triglyceride comprises short chain fatty acids, medium chain fatty acids, or a mixture thereof, further wherein in one aspect the triglyceride comprises butyrate and/or caprylate. Preferably the lipid is selected from:
   a. Triglycerides (TG) composed of a mixture of butyrate and medium chain fatty acids (MCFAs);
   b. Triglycerides (TG) composed of a mixture of short and medium chain fatty acids (SMCFAs);
   c. Triglycerides (TG) composed of a mixture of butyrate and long chain fatty acids (such as a mixture of butyrate and oleate);
   d. Triglycerides composed of a medium chain fatty acid;
   e. Triglycerides (TG) composed of a short chain fatty acid;
   f. Short chain fatty acids that could be metabolized into ketone bodies; and
   g. Short and/or Medium chain fatty acids that could be metabolized into ketone bodies, preferably C4 and/or C8 fatty acids (such as butyric acid (C4:0) and/or caprylic acid (C8:0)); and
   h. Triglycerides (TG) composed of butyrate and caprylate.

According to another aspect, the composition as defined herein is selected from of a food product, a food for special medical purposes (FSMP), a nutritional supplement, a dairy-based drink, a low-volume liquid supplement, a meal replacement beverage, and combinations thereof.

According to a second embodiment, there is the use of the composition as defined herein for lowering or avoiding accumulation of uremic toxins, preferably in the treatment of cardiometabolic or neurodegenerative conditions. The composition is thereby preferably used for the reduction of uremic toxins in cardiometabolic or neurodegenerative conditions and related comorbidities, for delaying the progression of such cardiometabolic or neurodegenerative conditions and comorbidities and/or for managing symptoms and syndrome associated with toxic effects of uremic solutes of such cardiometabolic or neurodegenerative conditions and related comorbidities.

Cardiometabolic or neurodegenerative conditions and related comorbidities that can be treated by using the composition particularly concern the following cases:
treatment or prevention of Kidney disease, including chronic and acute; dialysis and pre-dialysis states of kidney disease; rare (kidney) diseases, genetic- and metabolic-induced (kidney) diseases;
treatment or prevention of Uremic syndrome, including protein energy wasting syndrome, bone-loss, hyper anorexia, fatigue, or inflammation;
delay of advanced kidney disease complication, including anemia, hyperphosphatemia, secondary hyperparathyroidism, bone disease, and neurological conditions;
delay of kidney disease comorbidities, including cardiovascular disease;
prevention of the risk or management of malnutrition;
delay of the progression of cardiometabolic disease;
prevention of the risk or management of cardiovascular disease and comorbidities such as diabetes; and/or
prevention of the risk or management of neurodegenerative and neurological conditions.

According to a third embodiment, there is a method of treatment of cardiometabolic or neurodegenerative conditions as defined above, preferably comprising as a first step (a) a step of preparing and providing a composition as discussed above comprising the specifically selected probiotic, the specifically selected prebiotic and the specifically selected lipid as defined above; and (b) administering such a composition to a patient in need thereof, typically suffering from an increase of uremic toxins, typically in the context of cardiometabolic or neurodegenerative conditions as defined herein.

According to a fourth embodiment, there is a kit (of parts) suitable for use in lowering and/or avoiding accumulation of uremic toxins in cardiometabolic or neurodegenerative conditions as defined herein comprising the composition as described herein, e.g. comprising the specifically selected probiotic, the specifically selected prebiotic and the specifically selected lipid as defined above; for admixing to form one or more of the compositions disclosed herein and/or for use in one or more of the methods disclosed herein, for example in separate containers as two or more liquid solutions or dried powders.

### FIGURE DESCRIPTION

FIG. 1: shows a graphical representation of the biochemical pathways for formation of some of the intestinal bacterially-derived uremic toxins and their precursors. In this example, the generation of such as urea (NH3), p-cresyl sulfate (PCS), indoxyl sulfate (IS) and trimethylamine-N-oxide (TMAO) are shown with corresponding enzymes in Enzyme Code (EC) numbers. Uremic toxins precursors, such as p-cresol, indole and TMA are by-products of microbial metabolism of amino acids, such as Tryptophan, Tyrosine, Carnitine and Choline. These by-product metabolites are then further chemically modified in the liver where then they turn into uremic toxins, reaching the circulation and impacting target organs.

FIG. 2: shows a graphical representation for production and retention of uremic toxins in the body. FIG. 2(A) demonstrates how microbiome dysbiosis may contribute to build-up of uremic toxins in the systemic circulation leading to exacerbation of symptoms, comorbidities and progression of the disease. As shown in FIG. 2(B), uremic toxins negatively impact multitude of organs leading to clinical outcomes and manifestation of symptoms. FIG. 2B was adapted from Rosner et al., Clin J Am Soc Nephrol. 2021.

FIG. 3: shows an exemplary selection of probiotic strains for the purposes of the composition disclosed herein and treatments. As can be seen, the probiotic strains were selected already in an in silico screening process to lack different genes and key bacterial enzymes involved in uremic toxin metabolism. Lactobacillus johnsonii NCC 533 proved to lack the most number of relevant enzymes for avoiding production of uremic toxins or avoiding accumulation of uremic toxins and to provide most optimal basis for the envisaged treatment. White cell: enzymes not present; Gray cell: enzymes presence uncertain; Black cell: enzymes present. Tra: Tyramine; Trp: tryptophan; Tyr: tyrosine; indole-3-pyruvate: IPA; IAM: indole-3-acetamide; IAN: indole-3-acetonitrile; 4-HPPA: 4-Hydroxyphenylpyruvic acid.

FIG. 4: shows growth profiles of Lactobacillus johnsonii NCC 533 with different carbohydrate sources (alpha-galacto-oligosaccharides, galactomannans & fructans, which requires alpha-galactosidase and beta-fructo-furanosidase as enzymes encoded by the probiotic) during a 48-hour incubation. As can be seen, addition of Pea-GOS and Soy-GOS are most efficient, while addition of PHGG, Fenugreek, ScFOS and Inulin, although suitable, leads to smaller growth of Lactobacillus johnsonii NCC 533 (from top to bottom: soy GOS, pea GOS, Inulin, sFOS, PHGG, and Fenugreek). Growth tests using Lactobacillus johnsonii NCC 533 as a strain serve as examples only and can be applied to any of the further selected probiotics. Data were normalized with a negative control.

FIG. 5: shows growth profiles of Lactobacillus johnsonii NCC 533 with different carbohydrate sources (beta-galacto-oligosaccharides, which requires beta-galactosidase as enzymes encoded by the probiotic) during a 48-hour incubation. As can be seen, addition of BMOs and Vivianal GOS lead to particularly efficient growth of Lactobacillus johnsonii NCC 533 (from top to bottom: BMOs and Vivinal® GOS). Growth tests using Lactobacillus johnsonii NCC 533 as a strain serve as examples only and can be applied to any of the further selected probiotics. Data were normalized with a negative control.

FIG. 6: shows growth profiles of Lactobacillus johnsonii NCC 533 with different carbohydrate sources (cellooligosaccarides, which requires glucan 1,4-beta-glucosidase, cellulases and licheninase as enzymes encoded by the probiotic) during a 48-hour incubation. As can be seen, addition of Cellobiose and Cellotriose lead to particularly efficient growth of Lactobacillus johnsonii NCC 533 closely followed by Cellotetraose, soluble hydrolyzed wheat and soluble hydrolyzed oat (from top to bottom: Cellobiose, Cellotriose, Cellotetraose, soluble hydrolyzed wheat and soluble hydrolyzed oat). Growth tests using Lactobacillus johnsonii NCC 533 as a strain serve as examples only and can be applied to any of the further selected probiotics.

FIG. 7: shows an overview of the in-vitro/ex-vivo experimental set-up to assess the difference in the microbiome profile between healthy and Chronic Kidney Disease (CKD) donors using Prodigest's short-term single-stage colonic simulation technology.
FIG. 7(A) shows a sample schematic of the conditions and donor groups. For each donor group, two conditions ran in parallel: (1) standard condition consisting of basic nutrition food with minimal amino acid, mimicking amino acid content in low protein diet (2) AA spiked condition consisting of nutritional medium with a mixture of L-tryptophan, L-tyrosine, L-carnitine, choline and L-phenylalanine. The AA mixture was chosen as those are the substrates converted by the gut bacteria into uremic toxins and their precursors. AA concentration mimics the required amino acid intake for adult human per day. AA concentration mimics the required amino acid intake per day. A total of 9 healthy and 8 CKD donors were used in this example.
The timeline for experiments and sample analyses are shown in FIG. 7(B). Fecal microbiomes were inoculated in ProDigest's short-term single stage colonic system at day 0. From day 1 to day 2, the system was fed with basic nutrition food with or without AA mixture. Standard group fed with basic nutritional feed and AA spiked group fed with basic nutritional feed plus amino acid mixture. Samples were taken at different time points to measure overall fermentative activity, microbial community activity and microbiome community composition.
The readouts were as follows:
(1) Overall fermentative activity: acid/base consumption
(2) Microbial community activity:
   Lactate
   Short-chain fatty acids (SCFA): butyrate, propionate, acetate
   Markers of proteolytic activity: ammonium and branched SCFA (isobutyric acid, isovaleric acid and isocaprioic acid)
   Uremeic toxins & precursors: p-cresol, p.cresylsulfate, indole, indole-3-3acetic acid, betaine, trimethylamine, trimethylamine-N-oxide, indoxyl, indoxyl sulfate, semialdehyde glutaric acid, uric acid, urea
(3) Microbial community composition: quantitative deep shotgun sequencing

FIG. 8: shows an example of the difference in the microbiome metabolic capacity of the microbiome from Chronic Kidney Disease (CKD) patients and healthy donors. After 48-hour incubation in Prodigest's short-term single-stage colonic simulation system, CKD microbiome showed higher production of uremic toxin precursors, such as p-cresol, than healthy microbiome especially in the presence of excess amino acid substrates (FIG. 8(A)), suggesting a dysregulated amino acid metabolism of CKD microbiome. Moreover, CKD microbiome showed increased production of branched-chain fatty acids (BCFA) compared to healthy microbiome (FIG. 8(B)), suggesting higher proteolytic activity by CKD microbiome. As expected, byproducts of protein metabolism, such as BCFA, are not affected by excess amino acid substrates. Data represents the mean±SEM. *p<0.05 by Fisher's Least Significant Difference LSD (alpha=5%).

FIG. 9: shows an overview of the in-vitro/ex-vivo experimental set-up using a modified Prodigest's SHIME® technology to assess the impact of the new nutritional/symbiotic ingredients on CKD microbiome and the fecal microbiota from patients with Chronic Kidney Disease (CKD).
In particular, a sample schematic of the modified SHIME® system, consisting of UpperGIT vessel, which serves as stomach and small intestine, and colon vessel, which represents the transversal colon are shown in FIG. 9(A). For each donor, three conditions, consisting of 2 treatment groups and 1 control group, ran in parallel. In this example, a total of 8 Chronic Kidney Disease (CKD) donors were used.
Sample experimental timeline and sample analysis are depicted in FIG. 9(B). Fecal microbiomes from CKD donors were inoculated in ProDigest's SHIME system at day 0. From day 1 to day 10, the system was fed with basic nutrition food (with minimal amino acid, mimicking amino acid content in a low protein diet), with or without the nutritional/symbiotic blend interventions. In this example, two nutritional/symbiotic blend combinations (P1 and P2) were tested. P1 intervention consisted of an ingredient blend containing Lactobacillus johnsonii NCC533, cellobiose, short-medium chain triglycerides containing butyrate and caprylate. P2 intervention consisted of an ingredient blend containing Lactobacillus johnsonii NCC533, cellobiose, pea GOS, short-medium chain triglycerides containing butyrate and caprylate. During the last 2 days (from day 8 to day 10), the system was further challenged with additional amino acid (AA) mixture containing L-tryptophan, L-tyrosine, L-carnitine, choline and L-phenylalanine. The AA mixture was chosen as those are the substrates converted by the gut bacteria into uremic toxins and their precursors. Furthermore, AA concentrations were formulated to mimic the required amino acid intake for adult humans per day. Samples were taken at different time points to measure overall fermentative activity, microbial community activity and microbiome community composition.
The readouts were as follows:
(1) Overall fermentative activity: acid/base consumption
(2) Microbial community activity:
   Lactate
   Short-chain fatty acids (SCFA): butyrate, propionate, acetate
   Markers of proteolytic activity: ammonium and branched SCFA (isobutyric acid, isovaleric acid and isocaprioic acid)
   Uremeic toxins & precursors: p-cresol, p.cresylsulfate, indole, indole-3-3acetic acid, betaine, trimethylamine, trimethylamine-N-oxide, indoxyl, indoxyl sulfate, semialdehyde glutaric acid, uric acid, urea
(3) Microbial community composition: quantitative deep shotgun sequencing

FIG. 10: shows an example of the impact of the new nutritional or symbiotic blend on production of microbiota-derived uremic toxin precursors in-vitro/ex-vivo by the fecal microbiota from patients with Chronic Kidney Disease (CKD). As shown, interventions, labeled P1 and P2, significantly reduced the increased production of clinically relevant uremic toxin precursors by the CKD microbiome compared to the untreated control. Indole (A), p-cresol (B) and trimethylamine (C) are by-products of amino acid metabolism by the gut microbiota; thus, it follows that there is higher production in conditions where additional amino acid substrates are available. Notably, the interventions were effective in conditions with normal and minimal amino acid levels, thus suggesting the broad potential benefits of the intervention to CKD patients with different dietary requirements, such as low to high-protein diets. Overall, this data highlights the benefits of the invention to correct the amino acid dysmetabolism in the microbiome in CKD patients, leading to less uremic toxin build-up and associated clinical consequences. P1 intervention consisted of treating CKD microbiota with an ingredient blend containing Lactobacillus johnsonii NCC533, cellobiose and short-medium-chain triglycerides containing butyrate and caprylate while P2 intervention consisted of treating CKD microbiota with an ingredient blend containing Lactobacillus johnsonii NCC533, cellobiose, pea galacto-oligosaccharides and short-medium-chain triglycerides containing butyrate and caprylate. Control represents CKD microbiome that was left untreated. Data represents the mean±SEM. *p<0.05 by Student t-Test.

FIG. 11: shows an example of the impact of the new nutritional or symbiotic blend on production of microbiota-derived uremic toxins in-vitro/ex-vivo by the fecal microbiota from patients with Chronic Kidney Disease (CKD). As shown, interventions, labeled P1 and P2, significantly reduced the increased urea production by the CKD microbiome compared to the untreated control. Urea is a by-product of protein metabolism; thus, it follows that there is little impact on urea levels after the supplementation of additional amino acid. Overall, this data highlights the benefits of the invention to help improve uremic toxin build-up and associated clinical consequences, particularly in CKD patients. P1 intervention consisted of treating CKD microbiota with an ingredient blend containing Lactobacillus johnsonii NCC533, cellobiose and short-medium-chain triglycerides containing butyrate and caprylate while P2 intervention consisted of treating CKD microbiota with an ingredient blend containing Lactobacillus johnsonii NCC533, cellobiose, pea galacto-oligosaccharides and short-medium-chain triglycerides containing butyrate and caprylate. Control represents CKD microbiome that was left untreated. Data represents the mean±SEM. *p<0.05 by Student t-Test.

FIG. 12: shows the impact of the new nutritional or symbiotic blend on protein dysmetabolism by CKD microbiota from patients with Chronic Kidney Disease (CKD) in-vitro/ex-vivo. As shown, CKD microbiome treated with P1 and P2 interventions showed significantly lower levels of branched chain fatty acids (A) and ammonium (B), suggesting an improvement in overactive proteolytic activity that contributes to higher uremic toxin build-up. Proteolytic activity by gut microbiota is not significantly impacted by the supplementation of additional amino acid. Overall, this data highlights the benefits of the invention to correct dysbiosis in CKD microbiome and thus may help improve uremic toxin build-up. P1 intervention consisted of treating CKD microbiota with an ingredient blend containing Lactobacillus johnsonii NCC533, cellobiose and short-medium-chain triglycerides containing butyrate and caprylate while P2 intervention consisted of treating CKD microbiota with an ingredient blend containing Lactobacillus johnsonii NCC533, cellobiose, pea galacto-oligosaccharides and short-medium-chain triglycerides containing butyrate and caprylate. Control represents CKD microbiome that was left untreated. Data represents the mean±SEM. *p<0.05 by Student t-Test.

FIG. 13: shows the impact of the new nutritional or symbiotic blend on saccharolytic activity and the production of microbiota-derived beneficial metabolites by the fecal microbiota from patients with Chronic Kidney Disease (CKD) in-vitro/ex-vivo, particularly on short-chain fatty acid (SCFA) production. The gut microbiota may affect host metabolic health through microbial metabolites. The balance between producing microbial metabolites by saccharolytic and proteolytic fermentation may be an important determinant of metabolic health. Indeed, dysbiosis in Chronic Kidney Disease (CKD) patients has been characterized to be dominated by certain bacterial groups with a higher proteolytic to saccharolytic activity ratios. Amongst the best-studied saccharolytic microbial metabolites are the short-chain fatty acids (SCFA) acetate (A), propionate (B) and butyrate (C). SCFA have been shown to have multiple benefits to the host including but not exhaustive to the improvement of intestinal epithelial barrier function and inflammation. Increased gut permeability has been suggested to contribute to the increased availability of gut-derived uremic toxins and precursors in systemic circulation. Henceforth, increased production of metabolites, which may help improve the integrity of gut lining, may indirectly help to prevent excessive uremic toxins build-up in the systemic circulation, especially for CKD patients. As shown in the graph, P1 and P2 interventions showed significantly higher SCFA levels compared to non-treated control, suggesting the ability of the invention to correct the dysbiosis in CKD microbiome, leading to possible metabolic health benefits. P1 intervention consisted of treating CKD microbiota with an ingredient blend containing Lactobacillus johnsonii NCC533, cellobiose and short-medium-chain triglycerides containing butyrate and caprylate while P2 intervention consisted of treating CKD microbiota with an ingredient blend containing Lactobacillus johnsonii NCC533, cellobiose, pea galacto-oligosacharrides and short-medium-chain triglycerides containing butyrate and caprylate. Control represents CKD microbiome that was left untreated. Data represents the mean±SEM. *p<0.05 by Student t-Test.

FIG. 14: shows an overview of the animal experimental set-up to assess the impact of the new nutritional or symbiotic ingredients on uremic toxin production and chronic kidney disease (CKD) progression. The animal model that was used is the 5/6 nephrectomy animal model, which is one of the gold-standard and most frequently used rodent model in CKD research, including renal pharmaceutical research. Kidneys were ablated during 2-step surgery procedure to reduce kidney function (reminiscent of human CKD stage 3b and above). Additional animals were sham-operated and served as non-CKD animal control. From week 3 to week 10, the CKD animals were fed a diet with or without the nutritional or symbiotic blend interventions. In this example, two nutritional or symbiotic blend combinations (P1 and P2) were tested. P1 intervention consisted of an ingredient blend containing Lactobacillus johnsonii NCC533, cellobiose, short-medium chain triglycerides containing butyrate and caprylate. P2 intervention consisted of an ingredient blend containing Lactobacillus johnsonii NCC533, cellobiose, pea GOS, short-medium chain triglycerides containing butyrate and caprylate. Starting at week 8, metabolic parameters were evaluated including glucose tolerance test was performed. At week 9, mice were placed in metabolic cages to collect urine for uremic toxin and kidney parameter analysis. At week, 10, the mice were sacrificed and different tissues were collected for subsequent analysis. Tissue collection: plasma & serum, urine, liver heart, eWAT, scWAT, gastrocnemius muscle, soleus muscle, kidney all intestinal segments, caecum. Throughout the study, the physiological, behavioral and intake of food and water were monitored.
Endpoint Analysis
   Blood and urine concentrations of uremic toxins (e.g. urea, indoxyl sulfate, p-cresyl sulfate, TMAO, uric acid, CMPF, IAA, PCG)
   Assessment of kidney function—blood and urine renal markers (e.g. proteinuria, creatinine, albumin, Cystatin C), and kidney histopathology analysis
   Metabolic-inflammation markers (blood and tissue cytokines IL6, TNFa, IL1b); blood CRP; calprotectin and albumin in the feces)
   Intestinal function (epithelial function and permeability markers in blood such as citrulline; intestinal histology analysis; lipid/fat fecal levels)
   Improvement of CKD comorbidities (glucose regulation evaluated during oral glucose tolerance test, fasting glucose, insulin)
   Improvement of other metabolic parameters (plasma triglycerides, AST, ALT, cholesterol, LDL or HDL)
   Improvement of CKD comorbidities (adipose, liver, heart, gut and muscle will be collected for protein or RNA analysis)
   Cecal microbiome analysis—composition and function (metagenomics)
   Fecal, urine and serum untargeted metabolomics analysis

FIG. 15: shows the impact of the new nutritional or symbiotic blend on clinically relevant plasma uremic toxin levels in an animal model of Chronic Kidney Disease (CKD). As shown, animals that underwent nephrectomy (CKD group, represents animals with CKD, reminiscent of human CKD Stage 3b and above) showed higher uremic toxin production compared to non-CKD control animals (sham group, reminiscent of healthy individuals without CKD disease). P1 group is CKD animals treated with the intervention consisting of an ingredient blend containing Lactobacillus johnsonii NCC533, cellobiose, short-medium chain triglycerides containing butyrate and caprylate. P2 group is CKD animals treated with the intervention consisting of an ingredient blend containing Lactobacillus johnsonii NCC533, cellobiose, Pea GOS, short-medium chain triglycerides containing butyrate and caprylate. CKD animals treated with P1 and P2 interventions for 7 weeks showed significantly lower levels of plasma uremic toxins, such as p-cresyl sulfate (PCS) (A), indoxyl sulfate (IS) (B), P-cresylglucuronide (PCG) (C), Indole acetic acid (IAA) (D), 3-Carboxy-4-methyl-5-propyl-2-furanpropionate (CMPF) (E) and uric acid (F) compared to untreated CKD animals. The data clearly showed the benefit of the intervention to improve the build-up of uremic toxins in the systemic circulation, particularly for individuals with impaired kidney function. Each data points represent 1 animal. Line represents the mean. Multiple comparison was performed using ANOVA followed by uncorrected Fisher's LSD. *p<0.05, **p<0.01, ***p<0.001.

FIG. 16: shows the impact of the new nutritional or symbiotic blend on kidney parameters/markers of kidney function in an animal model of Chronic Kidney Disease (CKD). Sham group represents non-CKD animals, reminiscent of healthy individuals without CKD. CKD group represents animals with CKD, reminiscent of human CKD Stage 3b and above. P1 group is CKD animals treated with the intervention consisting of an ingredient blend containing Lactobacillus johnsonii NCC533, cellobiose, short-medium chain triglycerides containing butyrate and caprylate. P2 group is CKD animals treated with the intervention consisting of an ingredient blend containing Lactobacillus johnsonii NCC533, cellobiose, Pea GOS, short-medium chain triglycerides containing butyrate and caprylate. As shown, animals that underwent nephrectomy (CKD group) showed impaired kidney function demonstrated by increased (A) proteinuria, demonstrated by higher protein-to-creatinine ratio in the urine, and (B) increased concentration of urea in the plasma compared to non-CKD control animals (sham group, reminiscent of healthy individuals without CKD disease). CKD animals treated with P1 and P2 interventions for 7 weeks showed significantly better proteinuria and plasma urea levels compared to non-treated CKD animals. The data supports the benefits of the invention in stabilizing kidney function, and thus may be useful to help in preventing the vicious cycle of uremic toxin build-up and in slowing down the progression of kidney damage and related clinical outcomes. For proteinuria, each data point represents 2 animals whereas for urea levels, each data points represent 1 animal. Line represents the mean. Multiple comparison was performed using ANOVA followed by uncorrected Fisher's LSD. *p<0.05, **p<0.01, ***p<0.001.

FIG. 17: shows the impact of the new nutritional or symbiotic blend on kidney histology in an animal model of Chronic Kidney Disease (CKD). Sham group represents non-CKD animals, reminiscent of healthy individuals without CKD. CKD group represents animals with CKD, reminiscent of human CKD Stage 3b and above. P1 group is CKD animals treated with the intervention consisting of an ingredient blend containing Lactobacillus johnsonii NCC533, cellobiose, short-medium chain triglycerides containing butyrate and caprylate. P2 group is CKD animals treated with the intervention consisting of an ingredient blend containing Lactobacillus johnsonii NCC533, cellobiose, Pea GOS, short-medium chain triglycerides containing butyrate and caprylate. As shown, the remaining kidney of the animals that underwent nephrectomy (CKD group) showed microscopic damages, characterized by increased fibrosis (A) and impaired glomerular size (B) and volume (C), compared to non-CKD control animals (sham group, reminiscent of healthy individuals without CKD disease). CKD animals treated with P1 and P2 interventions for 7 weeks showed reduced fibrosis and glomerular size and volume. The data supports the benefits of the invention in reducing kidney microscopic damages, and thus may be useful to help in preventing the vicious cycle of uremic toxin build-up and in slowing down the progression of kidney damage and related clinical outcomes. Each data points represent 1 animal. Multiple comparison was performed using ANOVA followed by uncorrected Fisher's LSD. *p<0.05, **p<0.01, ***p<0.001. HES: Haemotoxylin and Eosin Staining.

FIG. 18: shows the impact of the new nutritional or symbiotic blend on (A) body weight evolution, (B) food intake and energy reserve wasting/protein energy wasting in animal model of Chronic Kidney Disease (CKD) disease. Some detrimental consequences of the build-up of uremic toxins in kidney disease are loss of appetite and loss of energy reserves including muscle and fat reserves. As shown in the figure, after the surgery, the animals that underwent nephrectomy (CKD group, represents animals with CKD, reminiscent of human CKD Stage 3b and above) showed slower body weight gain compared to non-CKD control groups (sham group, reminiscent of healthy individuals without CKD disease). Furthermore, CKD animals showed significantly lower food intake starting from week 2 all the way to week 10 compared to sham group. At the end of the study, (C) CKD animals further showed significantly reduced epididymal white adipose tissues (eWAT), suggesting reduced energy reserves reminiscent of what is seen in human CKD. CKD animals treated with the P1 and P2 intervention showed significantly improved body weight evolution and normalization of food intake to the same level as non-CKD sham animals. P1 group is CKD animals treated with the intervention consisting of an ingredient blend containing Lactobacillus johnsonii NCC533, cellobiose, short-medium chain triglycerides containing butyrate and caprylate. P2 group is CKD animals treated with the intervention consisting of an ingredient blend containing Lactobacillus johnsonii NCC533, cellobiose, Pea GOS, short-medium chain triglycerides containing butyrate and caprylate. Furthermore, treated animals, particularly those treated with P1 showed better adipose reserves as seen by improved epididymal white adipose tissue (eWAT) compared to non-treated CKD animals. The data supported the potential benefits of intervention to alleviate uremia-related symptoms including loss of appetite leading to weight loss and protein energy wasting. For the body weight graph, line represents the average of all animals per group with SEM as error bars. For the food intake, each data points represent 1 animal. Multiple comparison was performed using ANOVA followed by uncorrected Fisher's LSD. *p<0.05, **p<0.01, ***p<0.001.

FIG. 19: shows the impact of the new nutritional or symbiotic blend on intestinal barrier dysfunction in animal model of Chronic Kidney Disease (CKD). As shown, animals that underwent nephrectomy (CKD group, represents animals with CKD, reminiscent of human CKD Stage3b and above) showed impaired intestinal barrier, demonstrated by lower protein expression of the tight junction occluding in the ileum, compared to non-CKD control animals (sham group, reminiscent of healthy individuals without CKD disease). Tight junctions are specialized connection of two adjacent cell membranes, and in the case of gut lining, an important structure to prevent excessive translocation of gut-derived molecules, such as uremic toxin precursors, into the systemic circulation. CKD animals treated with P1 and P2 interventions for 7 weeks showed significantly higher protein expression of occludin compared to non-treated CKD animals. P1 group is CKD animals treated with the intervention consisting of an ingredient blend containing Lactobacillus johnsonii NCC533, cellobiose, short-medium chain triglycerides containing butyrate and caprylate. P2 group is CKD animals treated with the intervention consisting of an ingredient blend containing Lactobacillus johnsonii NCC533, cellobiose, Pea GOS, short-medium chain triglycerides containing butyrate and caprylate. The data supports the benefits of the invention in improving gut dysfunction, particularly those suffering with kidney impairments. Each data points represent 1 animal. Line represents the mean. Multiple comparison was performed using ANOVA followed by uncorrected Fisher's LSD. *p<0.05, **p<0.01, ***p<0.001.

FIG. 20: shows the impact of the new nutritional or symbiotic blend on (A) urine albumin, (B) urine creatinine, (C) urine protein (albumin) to creatine ratio, and (D) LOG urine protein (albumin) to creatine ratio at week 7 in a rat animal model of CKD. From left to right: sham, 5/6 Vehicle, 5/6 Nx P1-rat intervention (P1-rat intervention consisted of an ingredient blend containing Lactobacillus johnsonii NCC533 10⁸, cellobiose 1%, short-medium chain triglycerides containing butyrate and caprylate 1%), 5/6 Nx P3 intervention (intervention consisted of an ingredient blend containing Lactobacillus johnsonii NCC533 10⁸, cellobiose 0.3%, short-medium chain triglycerides containing butyrate and caprylate 0.3%), and 5/6 Nx Lisinopril 20 mg/kg. (A) Values expressed as mean of n=12-18+SEM. Dunnett's test one-factor linear model. **: P<0.01 compared to 5/6 Nx Vehicle and (B) Values expressed as mean of n=12-18+SEM. Dunnett's test one-factor linear model. **: P<0.01, ***: P<0.001 compared to 5/6 Nx Vehicle. Sham had a decreased level of Urine Albumin—Week 7 when compared to 5/6 Nx Vehicle. Sham had a decreased level of Urine Albumin—Week 7 when compared to 5/6 Nx Vehicle. (C) Values expressed as mean of n=12-18+SEM. Dunnett's test one-factor linear model. *: P<0.05, **: P<0.01 compared to 5/6 Nx Vehicle. (D) Values expressed as mean of n=12-18+SEM. Dunnett's test one-factor linear model. *: P<0.05, ***: P<0.001 compared to 5/6 Nx Vehicle. Sham and 5/6 Nx P1 had a decreased Urine ACR—Week 7 when compared to 5/6 Nx Vehicle. Sham and 5/6 Nx Lisinopril 20 mg/kg had a decreased LOG Urine ACR—Week 7 when compared to 5/6 Nx Vehicle.

FIG. 21: shows the plasma levels of (A) indoxyl sulfate (IS) and (B) p-cresyl sulfate (PCS) and (C) p-cresol glucuronide (PCG) in 5/6 Nx rats in the groups (from left to right) sham, 5/6 Vehicle, 5/6 Nx P3 intervention (intervention consisted of an ingredient blend containing Lactobacillus johnsonii NCC533 10⁸, cellobiose 0.3%, short-medium chain triglycerides containing butyrate and caprylate 0.3%), 5/6 Nx P1-rat intervention (indicated as “5/6 Nx P1” in FIG. 21(A)-(C)) (P1-rat intervention consisted of an ingredient blend containing Lactobacillus johnsonii NCC533 10⁸, cellobiose 1%, short-medium chain triglycerides containing butyrate and caprylate 1%) and 5/6 Nx Lisinopril 20 mg/kg. The 5/6 Nx P1-rat intervention reduced both IS an pCS plasma levels (~36% for IS and ~77% for pCS in comparison to 5/6 Nx Vehicle group).

### DETAILED DESCRIPTION OF THE INVENTION

### Definitions

Some definitions are provided hereafter are applicable for the specification and invention described. Nevertheless, definitions may be located in the “Background of invention”, in the section “Summary of invention”, in the “Examples” section below, and the following section “Definitions”.

All percentages expressed herein are by weight of the total weight of the composition unless expressed otherwise.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. A composition “consisting essentially of” contains at least 50 wt. % of the referenced components, preferably at least 75 wt. % of the referenced components, more preferably at least 85 wt. % of the referenced components, most preferably at least 95 wt. % of the referenced components.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. A food product typically includes at least one of a protein, a lipid, a carbohydrate and optionally includes one or more vitamins and minerals. The compositions of the present disclosure, including the many embodiments described herein, can comprise, consist of, or consist essentially of the elements disclosed herein, as well as any additional or optional ingredients, components, or elements described herein or otherwise useful in a diet.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The term “unit dosage form”, as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

A “subject” or “individual” is a mammal, preferably a human. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly individuals.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition disclosed herein, namely a composition comprising at least one probiotic bacteria, at least one prebiotic, and at least one lipid, all as defined herein. As used herein, “promoting” refers to enhancing or inducing relative to the level before administration of the composition disclosed herein.

The term “probiotic bacteria” means bacteria that are viable (live bacteria) and that provide health benefits when consumed, generally by improving or restoring the gut flora. The “probiotic bacteria” are preferably present in the composition in an effective amount. The manufacturing procedure of probiotic bacteria is typically standardized and involves a step of fermenting the bacteria in a growth medium comprising a carbohydrate source, such as a sugar, for example glucose, fructose, sucrose, lactose or dextrose. Following the fermentation, the probiotic bacteria are usually cryo-protected and frozen or freeze-dried and packaged into a finished product to be used in the composition. The “probiotic bacteria” as used according to the invention are specifically selected in view of the requirements to lower uremic toxins and preferably to allow avoiding or lowering an accumulation of such uremic toxins in the circulation, the tissues and organs as already depicted above.

The term “prebiotic” are typically to be understood to comprise non-digestible fiber compounds that pass undigested through the upper part of the gastrointestinal tract and stimulate the growth or activity of advantageous bacteria in the colon by acting as substrates for them. The prebiotics of the invention are specifically selected to allow stimulation of growth or activity of the “probiotic bacteria” as used according to the invention. The “prebiotics” are preferably present in the composition in an effective amount. The manufacturing procedure of probiotic bacteria is typically standardized and well known to a skilled person.

The term “Average Nucleotide Identity (ANI)” is a measure of nucleotide-level genomic similarity between the coding regions of two genomes. ANI can be readily determined by the skilled person using common knowledge and available tools, which are well detailed in the literature. For example, ANI can be assessed as describe here: Yoon S H, Ha S M, Lim J, Kwon S, Chun J. A large-scale evaluation of algorithms to calculate average nucleotide identity. Antonie Van Leeuwenhoek. 2017 October; 110(10):1281-1286.

As used herein, the term “cardiometabolic condition” refers to any condition involving a spectrum of conditions that are related to or share risk factors, such as overweight and obesity, dyslipidemia, and high blood pressure.

As used herein, the term “neurodegenerative condition” refers to any condition involving progressive loss of functional neurons in the central nervous system. In an embodiment, the neurodegenerative disease is associated with age-related cell death. Non-limiting examples of such neurodegenerative conditions include particularly, cardiometabolic or neurodegenerative conditions preferably concern the treatment or prevention of Kidney disease, including chronic and acute; dialysis and pre-dialysis; rare (kidney) diseases, genetic- and metabolic-induced (kidney) diseases; the treatment or prevention of Uremic syndrome, including protein energy wasting, bone-loss, hyper anorexia, fatigue, or inflammation; the delay of advanced kidney disease complication, including anemia, hyperphosphatemia, secondary hyperparathyroidism, bone disease, neurological conditions; the delay of kidney disease comorbidities, including cardiovascular disease; the prevention of the risk or management of malnutrition; the delay of the progression of cardiometabolic disease; the prevention of the risk or management of cardiovascular disease and comorbidities (diabetes); and/or the prevention of the risk or management of neurodegenerative and neurological conditions

Neurodegenerative conditions furthermore may include or be correlated with Alzheimer's disease, Parkinson's disease, Huntington's disease, amyotrophic lateral sclerosis (also known as ALS and as Lou Gehrig's disease), AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome.

“Sarcopenia” is defined as the age-associated loss of muscle mass and functionality (including muscle strength and gait speed). As used herein, “frailty” is defined as a clinically recognizable state of increased vulnerability resulting from aging-associated decline in reserve and function across multiple physiologic systems such that the ability to cope with everyday or acute stressors is compromised. In the absence of an established quantitative standard, frailty has been operationally defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with VO₂ max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females) and/or unintentional weight loss (10 lbs. in past year). Fried L P, Tangen C M, Walston J, et al., “Frailty in older adults: evidence for a phenotype.” J. Gerontol. A. Biol. Sci. Med. Sci. 56(3):M146-M156 (2001). A pre-frail stage, in which one or two of these criteria are present, identifies a high risk of progressing to frailty.

“Cachexia” is a severe body wasting condition characterized by marked weight loss, anorexia, asthenia, and anaemia. Cachexia is a common feature of a number of illnesses, such as cancer, sepsis, chronic heart failure, rheumatoid arthritis, and acquired immune deficiency syndrome (AIDS).

“Overweight” is defined for a human as a body mass index (BMI) between 25 and 30 kg/m². “Obese” is defined for a human as a BMI of at least 30 kg/m², for example 30-39.9 kg/m2. “Weight loss” is a reduction of the total body weight. Weight loss may, for example, refer to the loss of total body mass in an effort to improve one or more of health, fitness or appearance.

“Diabetes” encompasses both the type I and type II forms of the disease. Non-limiting examples of risk factors for diabetes include: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

As used herein, the term “metabolic syndrome” refers to a combination of medical disorders that, when occurring together, increase the risk of developing cardiovascular disease and diabetes. It affects one in five people in the United States and prevalence increases with age. Some studies have shown the prevalence in the United States to be an estimated 25% of the population. In accordance with the International Diabetes Foundation consensus worldwide definition (2006), metabolic syndrome is central obesity plus any two of the following:
Raised triglycerides: >150 mg/dL (1.7 mmol/L), or specific treatment for this lipid abnormality;
Reduced HDL cholesterol: <40 mg/dL (1.03 mmol/L) in males, <50 mg/dL (1.29 mmol/L) in females, or specific treatment for this lipid abnormality;
Raised blood pressure: systolic BP>130 or diastolic BP>85 mm Hg, or treatment of previously diagnosed hypertension; and
Raised fasting plasma glucose: (FPG)>100 mg/dL (5.6 mmol/L), or previously diagnosed type 2 diabetes.

### EMBODIMENTS

The present disclosure provides according to the first embodiment, a composition, preferably suitable for use in lowering and/or avoiding accumulation of uremic toxins, preferably in cardiometabolic or neurodegenerative conditions, comprising a specifically selected prebiotic, a specifically selected probiotic and a specifically selected lipid as defined herein.

The composition provides particular health benefits to a patient to be treated. Such a patient is typically a patient suffering from a cardiometabolic or neurodegenerative condition and is typically experiencing or at least being at the risk of experiencing an increase in and accumulation of uremic toxins. The health benefits, that can be advantageously provided to such a patient by administering the composition is preferably a delay in the progression of disease and comorbidities, and furthermore the possibility to manage symptoms and syndromes associated with toxic effects of such uremic solutes, both typically by lowering the amount of uremic toxins in such a patient, that otherwise would be accumulated in the patient.

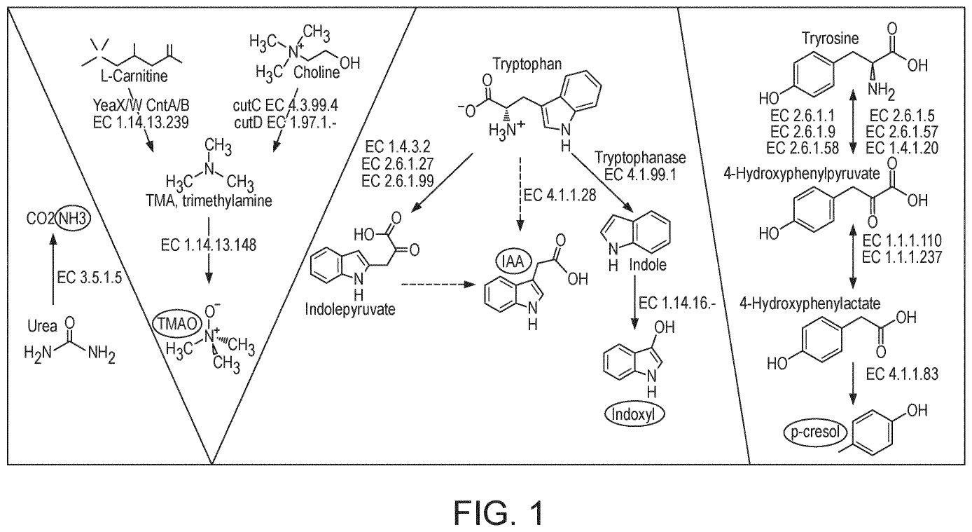

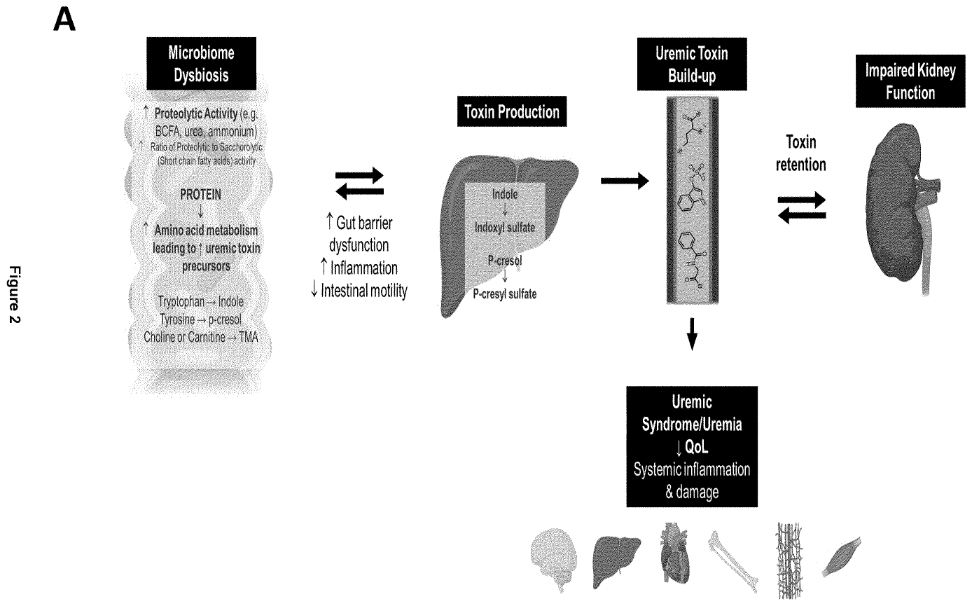

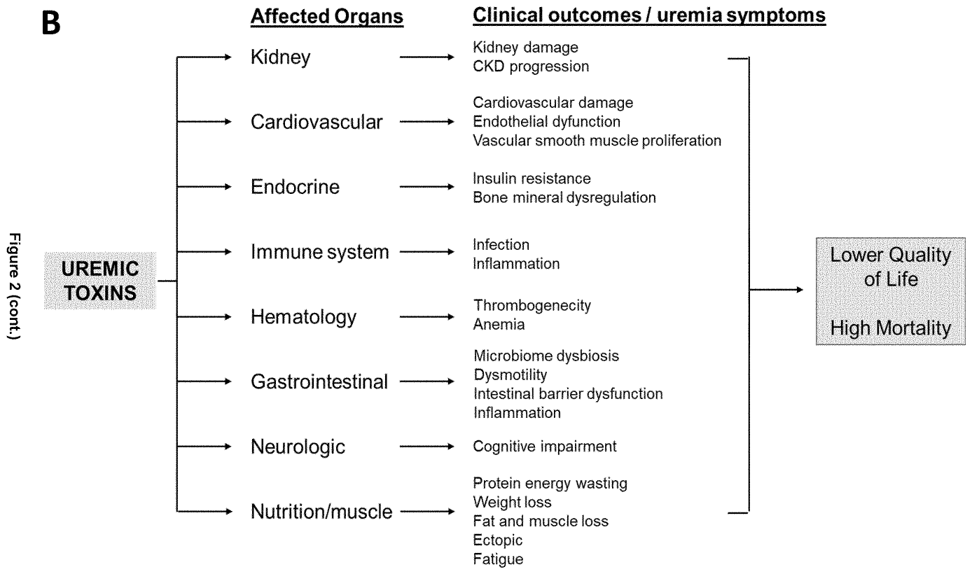

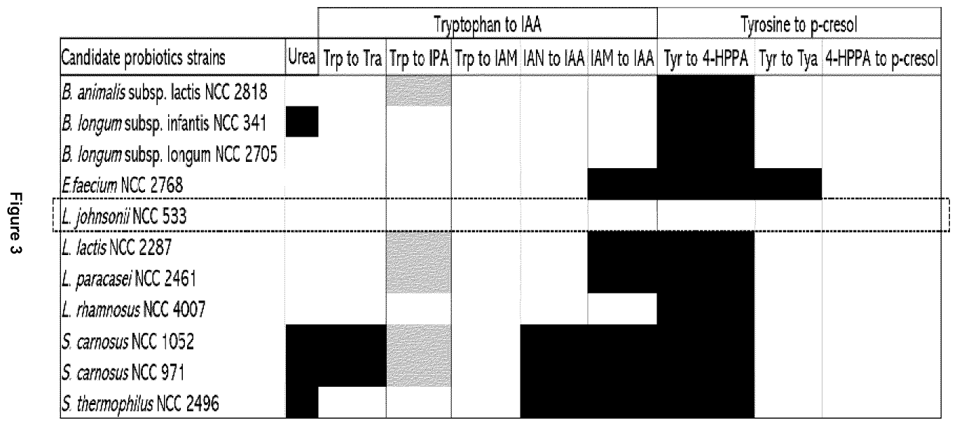

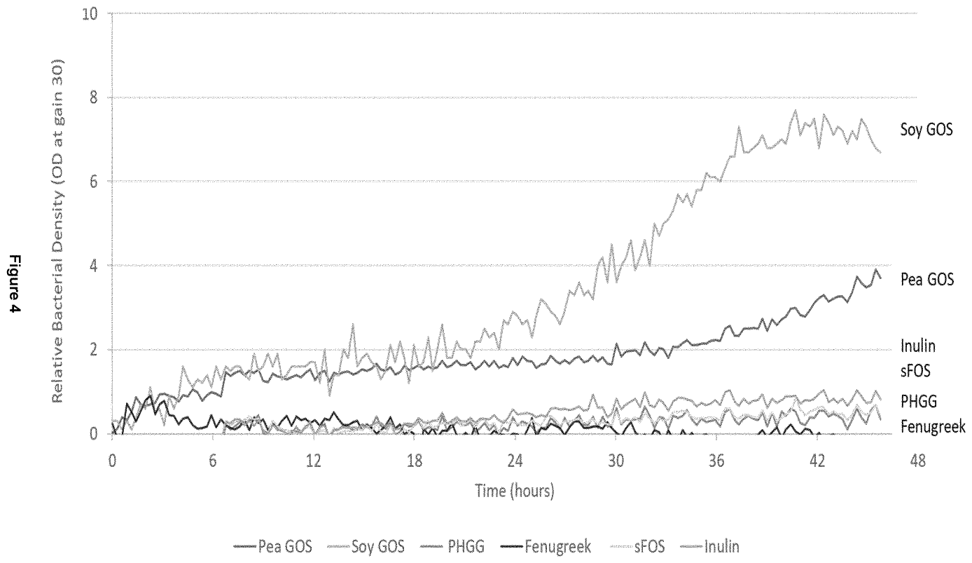

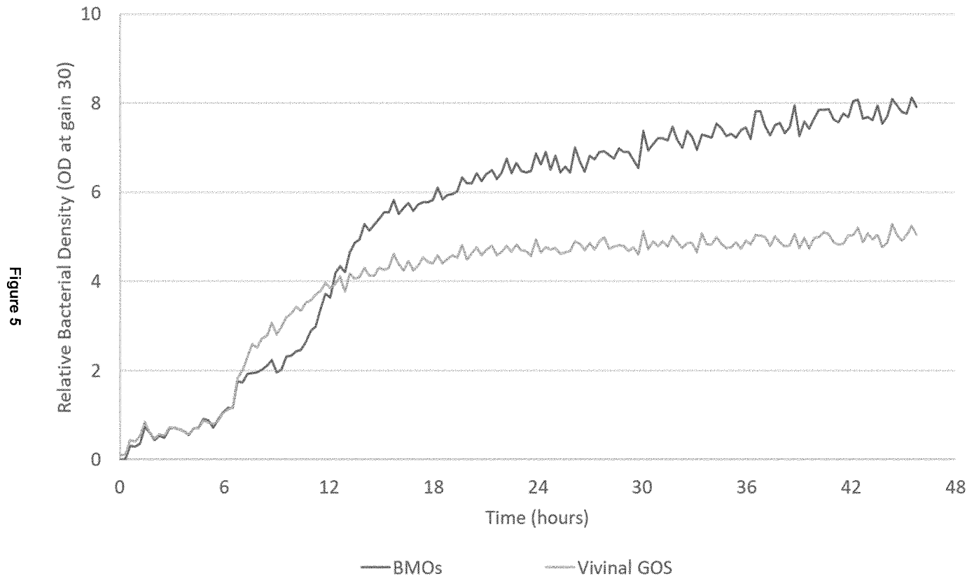

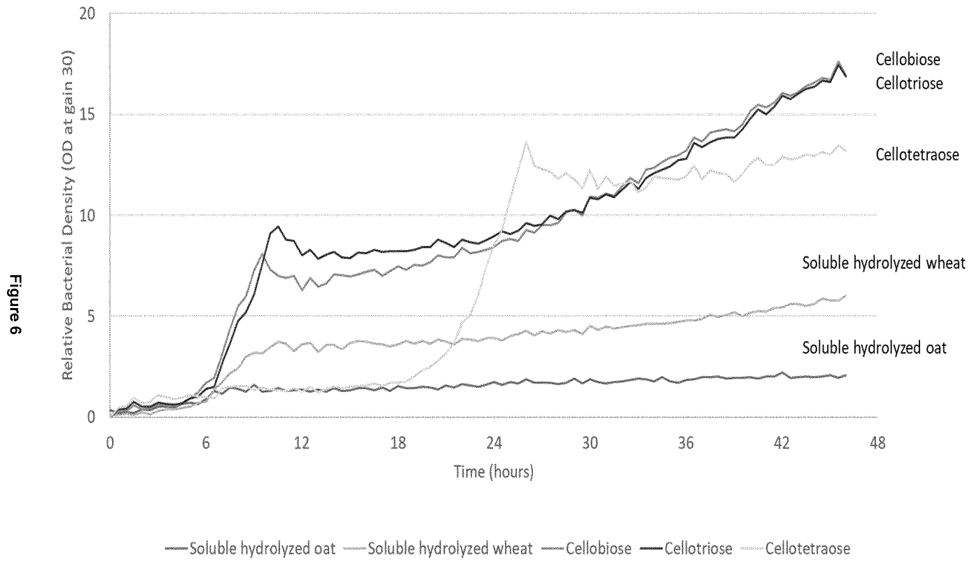

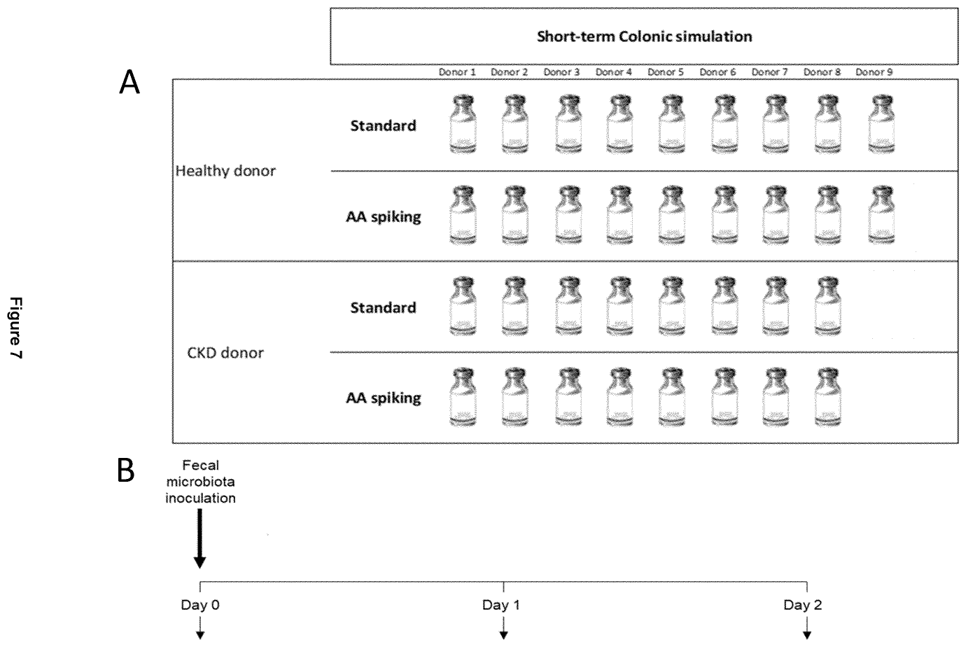

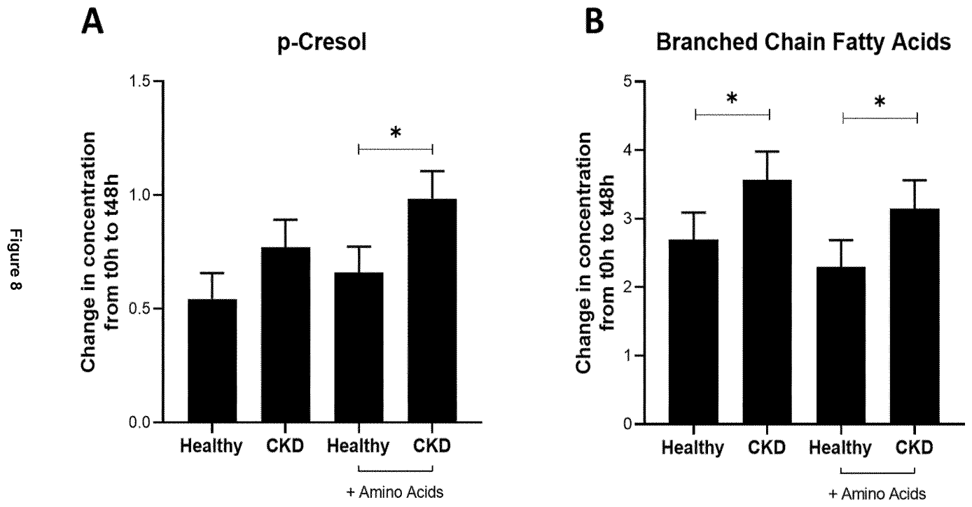

The uremic toxins within the context of the invention are typically selected from, but not limited to, urea, trimethylamine (TMA), Triethylamineoxide (TMAO), indoxyl and indoxyl sulfate, p-cresol and p-cresol sulfate, p-cresylglucorinade, uric acid, 3-Carboxy-4-methyl-5-propyl-2-furanpropionate (CMPF), etc. and all metabolic compounds or precursors thereof (confer e.g. to FIG. 1). Such uremic toxins mainly have their origin in the provision of either an excess in amino acids to a patient, such as tryptophan or tyrosine, increased amounts of L-carnitine or choline, and increased amounts of urea. An accumulation of such systemic uremic toxins is a condition often observed in individuals with cardiometabolic and neurological conditions, including but not limited to chronic kidney disease (CKD), and diseases and conditions as described herein above. An accumulation of such systemic uremic toxins also sets a high pressure on the treatment of such diseases, particularly in cases a high protein diet would be required, since the further addition of proteins, such as in muscle wasting conditions, such as cachexia or sarcopenia and various neurological conditions, would be rather detrimental due to the addition of further proteins and particularly the addition of further tryptophan and tyrosine.

In a first embodiment is provided a composition, representing a new nutritional blend and/or symbiotic blend that allows targeting multiple direct and indirect mechanisms that could contribute to the beneficial reduction of the generation and/or the accumulation of such uremic toxins, particularly by applying a composition representing preferably a specific blend composed of
specifically selected probiotics as defined herein (e.g. L. johnsonii NCC 533)
specifically selected prebiotics as defined herein, preferably fibers and oligosaccharides, as defined herein (e.g. cellobiose and pea GOS, etc.)
specifically selected lipids as defined herein, preferably triglycerides composed of short & medium chain fatty acids, as defined herein;

The composition, e.g. in form of a nutritional blend and/or symbiotic blend, positively targets and improves the microbiome (function), the gut function, the liver metabolism and provides mechanisms to stabilize the kidney function and helps lowering the systemic occurrence and amount of uremic toxins.

Preferably, the microbiome (function) can be improved thereby as follows: The microbiome usually contributes to the production of certain metabolites, which form precursors for uremic toxins. When such metabolites are available in the body tissues, particularly liver and circulation, they could be converted into uremic toxins. Importantly, patients with kidney disease have been shown to have dysbiosis, which is characterized by an impaired microbiome function to convert certain amino acids from dietary proteins into uremic toxin precursor metabolites. Thereby, administration of the composition improves excess availability of uremic toxins precursors in the circulation by improving dysbiosis, particularly microbiota function. In other words, the imbalance of the microbiome is improved, thereby allowing to also improve the metabolic processing of such uremic toxin precursor metabolites.

The composition, furthermore advantageously allows also increasing or improving the gut function. In this context, it is important to realize that the microbiota from both healthy and diseased individuals constantly and normally produce uremic toxins precursors without leading to overt disease outcome. However, patients with cardiometabolic diseases, particularly kidney diseases, may have an impaired gut function including impaired gut motility, gut epithelial barrier function and increased inflammation, which could contribute to dysregulated gut accumulation and increased permeability across the gut barrier of uremic toxin precursors. The composition thereby improves the availability of uremic toxin precursors in the systemic circulation by improving gut motility and epithelial barrier function and thereby significantly contributes to an avoidance of accumulation of such uremic toxins in the tissue and organs, particularly the kidneys.

The composition also provides significant benefits on liver metabolism. The liver is an integral tissue responsible in producing uremic toxins. Thereby, liver enzymes use substrate coming from endogenous source or other tissues, diet and gut microbiota to produce such uremic toxins. In this particular context, the composition allows improving metabolism leading to uremic toxins conversion by specific preselection of probiotics as well as of prebiotics and lipids to be administered, especially when substrate availabilities are in excess. In return, this improves uremic toxin accumulation in the circulation by improving liver metabolism and thereby effectively allows avoiding an undesired accumulation of these uremic toxins in the circulation and the liver.

The composition also provides significant benefits on the stabilization of kidney function. In a normal healthy situation, the kidney is able to filter out the uremic toxins and thereby to lower or avoid an accumulation of uremic toxins in blood circulation. However, in situations wherein a problem in proper kidney filtration occurs, uremic toxins can accumulate and initiate or contribute to a vicious disease loop cycle of exacerbation and progression, including further induction of dysbiosis or liver or gut impairments. The composition thereby allows for stabilizing kidney function and preventing a further decline. The composition therefore directly improves uremic toxin accumulation and indirectly improves uremic toxins production.

The invention is therefore based on a novel combination of specifically selected probiotics, specifically selected prebiotics, and specifically selected lipids, which in combination symbiotically allows improving levels of and avoiding accumulation of uremic toxins (e.g. indoxyl sulfate, p-cresyl sulfate, PCG, CMPF, uric acid) in different cardiometabolic (e.g. kidney diseases) and neurodegenerative conditions.

In the context of the invention, such an avoidance of the accumulation of uremic toxins particularly concerns compounds urea, uric acid, p-cresol, p-cresyl sulfate, indoxyl sulfate, indole, indole acetic acid, indoxyl, trimethylamine, TMAO, dimethylglycine, betaine, PCG, CMPF, glutarate and/or other protein-bound uremic toxin, preferably indoxyl sulfate, p-cresyl sulfate, MCPF, PCG, urea and/or uric acid. Measurement of such compounds in the tissues, organs and body fluids is well known to a skilled person.

In this context, the inventors have surprisingly found that the compositions, which comprise all of the herein required ingredients, namely the specifically selected probiotics, the specifically selected prebiotics and the specifically selected lipids, do not only allow addressing isolated aspects, such as dysbiosis or improvement of microbiome function but represents a holistic approach to mitigate multiple targets known to contribute in both production and accumulation of uremic toxins, particularly to improve the microbiome (function), the gut function, the liver metabolism and provides mechanisms to stabilize the kidney function and to help lowering the systemic occurrence and amount of uremic toxins, preferably in cardiometabolic or neurodegenerative conditions as defined herein.

The composition comprises probiotics as already defined above. Probiotics are considered as defined herein as live bacteria that contribute beneficially to gut and microbiome function. The probiotic bacteria of the composition provide a significant impact on this treatment and are selected according to the following three criteria, which may be applied separately or in combination, preferably in combination.

According to a first criterion such probiotic bacteria are selected from at least one of those probiotic bacteria lacking at least one bacterial enzymes to produce urea, uric acid, p-cresol, p-cresyl sulfate, indoxyl sulfate, indole, indole acetic acid, indoxyl, trimethylamine, TMAO, dimethylglycine, betaine and/or glutarate.

The lack of at least one bacterial enzyme as depicted above is typically achieved by a corresponding partial or complete lack or at least inactivation of the genetic coding sequence for such an enzyme in the genome of the probiotic bacterium, leading to a lack of expression of such an enzyme in vivo. If multiple copies of the bacterial enzyme are encoded by the genomic sequence, only a part or all, preferably all coding sequences of the bacterial enzyme are lacking or at least inactive.

The avoidance of the expression of such enzymes that are required to produce such uremic toxins allows lowering the otherwise possibly too high increase and load of uremic toxins in the body, tissue and organs, that potentially leads to a non-reversible accumulation of such toxins. Although the selected probiotic bacteria are bacteria generally considered beneficial in the gut health system, it was surprising for the inventors to recognize that uremic toxins can be efficiently lowered or at least limited by selecting a specific type of probiotics, prebiotics and lipids in a symbiotic manner. The probiotic bacteria used in the composition are particularly selected for certain properties, such as probiotic bacteria lacking specific bacterial enzymes that might contribute to an excess increase of uremic toxins (1^{St} and 2^{nd} alternative below) and/or that contribute to an improved growth of the probiotic bacteria by promoting expression of particular enzymes beneficial for processing specific prebiotics (fibers and carbohydrates).

According to a second criterion, preferably additionally to the first criterion, such probiotic bacteria are selected from at least one probiotic bacterium lacking at least one bacterial enzyme selected from urease, carnitine monooxygenase & reductase, tryptophanase, hydroxyphenylacetate. Such enzymes may be preferably selected from one of the following classes of enzymes:
EC1.14.13.239, EC: 1.14.12.17, EC: 1.14.13.25, EC: 1.14.99.-, EC: 1.3.3.4, EC: 1.3.8.2, EC: 1.3.98.1, EC: 1.4.1.13, EC: 1.4.1.14, EC: 1.4.3.2, EC: 2.1.1.12, EC: 2.3.1.54, EC: 2.6.1.-, EC: 2.6.1.1, EC: 2.6.1.2, EC: 2.6.1.2, EC: 2.6.1.4, EC: 2.6.1.44, EC: 2.6.1.5, EC: 2.6.1.57, EC 2.6.1.58, EC: 2.6.1.78, EC: 2.6.1.79, EC: 2.6.1.9, EC: 3.2.1.172, EC: 3.5.1.111, EC: 3.5.1.3, EC: 3.5.1.4, EC: 3.5.5.1, EC: 3.6.1.3, EC: 4.1.1.105, EC: 4.1.1.16, EC: 4.1.1.25, EC: 4.1.1.25, EC: 4.1.1.28, EC: 4.1.99.1, EC: 4.3.99.4, EC: 4.4.1.8, EC: 5.4.99.9, EC: 6.3.5.-, EC: 6.3.5.1, EC: 6.3.5.7, EC:1.13.11.63, EC:1.13.12.3, EC:1.14.13.148, EC:1.14.13.239, EC:1.18.1.2, EC:1.3.3.4, EC:1.4.1.20, EC:1.4.1.4, EC:1.4.3.2, EC:1.5.1.34, EC:1.8.1.19, EC:1.97.1, EC:2.6.1.-, EC:2.6.1.1, EC:2.6.1.39, EC:2.6.1.57, EC:2.6.1.78, EC:2.6.1.79, EC:2.6.1.9, EC:3.5.1.3, EC:3.5.2.12, EC:4.1.1.15, EC:4.1.1.83, EC:4.4.1.8

As already outlined for the first criterion, the lack of at least one such bacterial enzyme as depicted above is typically achieved by a corresponding partial or complete lack or at least inactivation of the genetic coding sequence for such an enzyme in the genome of the probiotic bacterium, leading to a lack of expression of such an enzyme in vivo. If multiple copies of the bacterial enzyme are encoded by the genomic sequence, only a part or all, preferably all coding sequences of the bacterial enzyme are lacking or at least inactive.

Furthermore, the lack at least one bacterial enzymes urease, carnitine monooxygenase & reductase, tryptophanase, hydroxyphenylacetate efficiently contributes to an avoidance of the further accumulation of uremic toxins that otherwise would be provided in excess and further accumulated in the body, tissues and organs of a patient above tolerable levels.

Alternatively or additionally to any of the aforementioned two criteria, preferably additionally, such probiotic bacteria are preferably selected according to a third criterion from at least one probiotic bacteria possessing at least one of the following bacterial carbohydrate enzymes: α-galactosidase, β-galactosidase, glucan 1,4-β-glucosidase, cellulase, β-fructofuranosidase and/or Licheninase. Such enzymes may be selected from one of the following classes of enzymes:
EC3.2.1.22, EC3.2.1.23, EC3.2.1.74, EC 3.2.1.4, EC 3.2.1.26, EC 3.2.1.58, EC 3.2.1.73, EC 2.4.1.9, EC 3.2.1.84, EC 3.2.1.33, EC 3.2.1.70, EC 3.2.1.135, EC 3.2.1.3, EC 3.2.1.20, EC 3.2.1.10

Expressing at least one of these bacterial carbohydrate enzymes via the specifically selected probiotics particularly contributes to a processing the herein administered prebiotics, which symbiotically (in view of the selections for the probiotics and the prebiotics) supports growth of the administered probiotics, improvement of microbiome and gut functions as well as liver metabolism and stabilization of kidney function, and thereby lowering of and/or avoidance of accumulation of uremic toxins.

The probiotic bacterium of the composition meets at least one, preferably at least two, more preferably at least three, or all, of the above mentioned criteria for inventive probiotics.

As already outlined before, such a probiotic bacterium according to any of such criteria, preferably meeting all three criteria, may be preferably selected from the following species Bifidobacterium animalis subspecies lactis, Bifidobacterium longum subspecies infantis, Bifidobacterium longum subspecies longum, Enterococcus faecium, Lactobacillus johnsonii, Lactococcus lactis, Lacticaseibacillus paracasei (previously classified as Lactobacillus paracasei), Limosilactobacillus reuteri (previously classified as Lactobacillus reuteri), Lacticaseibacillus rhamnosus (previously classified as Lactobacillus rhamnosus), Staphylococcus carnosus and/or Streptococcus thermophiles, preferably, Lactobacillus johnsonii, or a probiotic bacterium having a genome that has at least 95% Average Nucleotide Identity (ANI), preferably at least 96% ANI, more preferably at least 97% ANI, even more preferably at least 98% ANI, most preferably at least 99% ANI or even 99.5% ANI or 99.9% ANI to the genomic sequence of any one of the probiotic bacteria as defined before.

Even more preferably, such a probiotic bacterium preferably meeting all three criteria may be selected from any of the following strains:
a. Bifidobacterium animalis subspecies lactis NCC 2818, which was deposited on 7 Jun. 2005 with CNCM and assigned accession number CNCM I-3446;
b. Bifidobacterium longum subspecies infantis NCC 341 (ATCC 15697 (T)—which is available from worldwideweb.atcc.org/products/15697]);
c. Bifidobacterium longum subspecies longum NCC 2705 (CNCM I-2618?(NCBI refseq; GCA_000007525.1) [NCC 2075 was deposited on 29 Jan. 2001 with CNCM and assigned accession number CNCM I-2618];
d. Enterococcus faecium NCC 2768 (NCIMB 10415 which is available from Cerbios-Pharma SA Barbengo Switzerland (cerbios.swiss/e-faecium-sf68-a-model-for-efficacy-safety-for-pharmaceutical-probiotics/);
e. Lactobacillus johnsonii NCC 533 [originally known as La 1 which was deposited on 30 Jun. 1992 with CNCM and assigned accession number CNCM I-1225)(see also NCBI refseq; GCA_000008065.1, and GenBank AE017198.1 (included as SEQ ID NO: 1))]; f. Lactococcus lactis NCC 2287 (CNCM I-4154) [NCC 2287 was deposited on 24 Apr. 2009 with CNCM and assigned accession number CNCM I-4154];
g. Lacticaseibacillus paracasei NCC 2461 (CNCM I-2116) [NCC 2461 was deposited on 12 Jan. 1999 with CNCM and assigned accession number CNCM I-2116];
h. Lacticaseibacillus rhamnosus NCC 4007 (CGMCC 1.3724) [NCC 4007 was deposited in October 2004 with identification number CGMCC 1.3724 with CGMCC [China General Microbiological Culture Collection Center (CGMCC) Institute of Microbiology, Chinese Academy of Sciences, P.O. Box 2714, Beijing 100080, China];
i. Staphylococcus carnosus NCC 1052 (CNCM I-5400) [NCC 1052 was deposited on 1 Feb. 2019 with CNCM and assigned accession number CNCM I-5400];
j. Staphylococcus carnosus NCC 971 (CNCM I-5398) [NCC 971 was deposited with CNCM on 1 Feb. 2019 and assigned accession number CNCM I-5398]; and/or;
k. Streptococcus thermophilus NCC 2496 (CNCM I-3915) [NCC 2496 was deposited with CNCM on 5 Feb. 2008 and assigned accession number CNCM I-3915];
   or a probiotic bacterium having a genome that has at least 95% Average Nucleotide Identity (ANI), preferably at least 96% ANI, more preferably at least 97% ANI, even more preferably at least 98% ANI, most preferably at least 99% ANI or even 99.5% ANI or 99.9% ANI to the respective genomic sequences according to any one of a. to k. As defined above.

Most preferably, the probiotic bacteria of the composition as defined herein is selected from Lactobacillus johnsonii NCC 533 (CNCM I-1225) or a probiotic having a genome that has at least 95% ANI, preferably at least 96% ANI, more preferably at least 97% ANI, even more preferably at least 98% ANI, most preferably at least 99% ANI or even 99.5% ANI or 99.9% ANI to the genomic sequence of Lactobacillus johnsonii NCC 533 (CNCM I-1225). The NCBI reference sequence of Lactobacillus johnsonii NCC 533 (CNCM I-1225) is GCA_000008065.1 (SEQ ID NO: 1).

Typically, an “effective amount” of a “probiotic bacteria” as defined herein may comprise such a probiotic bacteria typically in an amount of between 10³ cfu to 10¹² cfu, typically in an amount of between 10⁴ cfu to 10¹¹ cfu per daily dose, preferably in an amount of between 10⁵ cfu to 10¹⁰ cfu per daily dose, or 10⁵ cfu to 10⁹ cfu per daily dose, likewise preferably in an amount of between 10⁶ cfu to 10⁹ cfu per daily dose, 10⁶ cfu to 10⁸ cfu per daily dose or in an amount of 10⁸ cfu to 10¹⁰ cfu per daily dose, more preferably around 10⁷ cfu to 10⁹ cfu per daily dose. A preferred daily dose is around 10⁸ total cfu per daily dose, 10⁷ to 10⁹ cfu per daily dose or 10⁸ to 10⁹ cfu per daily dose. A daily dose may thereby be achieved by a single administration of the composition per day or by multiple administrations of the composition, e.g. by two, three, four or five administrations, preferably not more than 1 to 5, more preferably not more than 1 to 4, even more preferably not more than 1 to 3 administrations. In case the daily dose is achieved by a single administration, or 1-5 administrations of the composition per day the amounts per composition are recalculated based on the required daily dose. It is preferred that in case of such multiple daily administrations any administration or respective composition comprises the same amount of ingredients and hence probiotics, prebiotics and lipids, and preferably also the same volume.

According to the second aspect, the composition as defined herein also comprises a prebiotic as already defined generally above. Such a prebiotic is selected from carbohydrates, preferably fibers and oligosaccharides, wherein the carbohydrate can be hydrolyzed by at least one bacterial enzyme selected from α-galactosidase, β-galactosidase, glucan 1,4-β-glucosidase, cellulase, β-fructofuranosidase and/or Licheninase, preferably selected from
EC 3.2.1.22, EC 3.2.1.23, EC 3.2.1.74, EC 3.2.1.4, EC 3.2.1.26, EC 3.2.1.58, EC 3.2.1.73, EC 2.4.1.9, EC 3.2.1.84, EC 3.2.1.33, EC 3.2.1.70, EC 3.2.1.135, EC 3.2.1.3, EC 3.2.1.20, EC 3.2.1.10.

Likewise, such a prebiotic is selected from the family of α-galacto-oligosaccharides/raffinose (e.g. pea GOS, soy GOS), β-galacto-oligosaccharides (e.g. β-GOS, Vivinal GOS, bovine milk oligosaccharides (BMOS)), human milk oligosaccharides (HMOs), cello-oligosaccharides (COS) (e.g. cellobiose, cellotriose, cellotetraose, soluble hydrolyzed wheat, soluble hydrolyzed oat and/or β-glucan hydrolysates containing cellobiose or cellotriose or cellotetraose), or a combination thereof.

The specific selection of the prebiotic in the composition symbiotically acts together with the specific selection of a probiotic bacteria of the composition as defined before. Such prebiotics are specific carbohydrates, preferably fibers and oligosaccharides, that preferably and beneficially support growth of the specifically selected probiotic bacteria. More specifically, the specific carbohydrates act as substrates of the specifically selected probiotic bacteria and can be hydrolyzed by at least one bacterial enzyme selected from α-galactosidase, β-galactosidase, glucan 1,4-β-glucosidase, cellulase, β-fructofuranosidase and/or Licheninase, produced by the probiotic bacteria of the composition as defined before due to the specific selection of the probiotic bacteria.

According to a further aspect the prebiotics are preferably contained in the composition as defined herein in an effective amount. More preferably, such an effective amount of prebiotics preferably contained in the composition as defined herein is an amount of between 0.1 g to 30 g per daily dose, preferably in an amount of between 2 g to 15 g per daily dose.

According to the third aspect the composition comprises in addition to the specifically selected probiotics and the specifically selected prebiotics as depicted before also specifically selected lipids.

Such lipids comprise at least one lipid selected from a triglyceride, a short chain fatty acid and/or a medium chain fatty acid, wherein the triglyceride comprises short chain fatty acids, medium chain fatty acids, long chain fatty acids, or a mixture thereof. Preferred triglycerides include a triglyceride comprising butyrate and/or caprylate, a triglyceride consisting of butyrate and caprylate, a triglyceride consisting of butyrate and oleate. In one aspect, the triglyceride consists of butyrate and caprylate. The inventors have surprisingly found that these specifically selected lipids advantageously support improvement of the microbiome, gut function, liver metabolism and stabilization of kidney function in a symbiotic manner and thereby significantly contribute to avoidance of both production and accumulation of uremic toxins.

In this context “short chain triglycerides” (SCTs) are preferably understood as triglycerides with two or three fatty acids each having 1 to 5 carbon atoms, preferably having 2 to 5 carbon atoms. Such fatty acids having 1 to 5 carbon atoms, preferably 2 to 5 carbon atoms, are generally understood herein as short chain fatty acids (SCFAs). A particularly preferred short chain fatty acid is a C4 fatty acid, most preferably butyric acid (C4:0). A short chain triglyceride comprising butyric acid is preferably an SCT that comprises at least one, two or even three butyric acids.

Likewise, in this context “medium chain triglycerides” (MCTs) are preferably understood as triglycerides with two or three fatty acids each having 6 to 12 carbon atoms. Such fatty acids having 6 to 12 carbon atoms are generally understood as medium chain fatty acids (MCFAs). A preferred short chain fatty acid is C8 fatty acid, such as. caprylic acid (C8:0). A medium chain triglyceride comprising caprylic acid may be an MCT that comprises at least one, at least two or even three caprylic acids.

In the context of a triglyceride comprising a mixture of butyrate and long chain fatty acids, it is preferably understood as a triglyceride with one or two butyrate moieties and one or two long chain fatty acids. A long chain fatty acid is generally understood to be a fatty acid is a fatty acid having 13 to 21 carbon atoms. Exemplary triglycerides comprising butyrate and a long chain fatty acid include 1,3-dibutyryl-2-linoleoylglycerol, 1,3-dibutyryl-2-stearoylglycerol, 1-butyryl-2-oleoyl-3-palmitoylglycerol, 1-palmitoyl-2-oleoyl-3-butyrylglycerol, 1-butyryl-2-oleoyl-3-linoleoylglycerol, 1-linoleoyl-2-oleoyl-3-butyrylglycerol, 1-oleoyl-2-butyryl-3-linoleoylglycerol, 1-linoleoyl-2-butyryl-3-oleoylglycerol, 1-butyryl-2-linoleoyl-3-oleoylglycerol, 1-oleoyl-2-linoleoyl-3-butyrylglycerol, 1-butyryl-2-stearoyl-3-oleoylglycerol, 1-oleoyl-2-stearoyl-3-butyrylglycerol, 1-butyryl-2-oleoyl-3-stearoylglycerol, and/or 1-stearoyl-2-oleoyl-3-butyrylglycerol, and mixtures of two or more thereof. A preferred long chain fatty acid in the context of a triglyceride comprising a mixture of butyrate and a long chain fatty acid is oleic acid (C18:1).

More preferably, a lipid in the context of the composition is selected from a. Triglycerides (TG) composed of a mixture of butyrate and medium chain fatty acids (MCFAs); b. Triglycerides (TG) composed of a mixture of short and medium chain fatty acids (SMCFAs); c. Triglycerides (TG) composed of a mixture of butyrate and long chain fatty acids (such as a mixture of butyrate and oleate); d. Triglycerides composed of a medium chain fatty acid; e. Triglycerides (TG) composed of a short chain fatty acid; f Short chain fatty acids that could be metabolized into ketone bodies; and/or g. Medium chain fatty acids that could be metabolized into ketone bodies, preferably C4 and/or C8 medium chain fatty acids (such as butyric acid (C4:0) and/or caprylic acid (C8:0)). Exemplary triglycerides in the context of the composition are a triglyceride comprising butyrate and/or caprylate, a triglyceride consisting of butyrate and caprylate, and a triglyceride consisting of butyrate and oleate. In one aspect, the triglyceride consists of butyrate and caprylate.

Particularly preferred as lipid in the context of the composition are triglycerides comprising butyrate, short chain fatty acids, medium chain fatty acids, or a mixture thereof. Such a lipid may be therefore
a triglyceride, containing at least one short chain fatty acid C1-C5, preferably C2-C5;
preferably a triglyceride, containing at least one butyric acid (C4:0), or a
triglyceride containing at least one medium chain fatty acid C6-C12, such as caprylic acid (C8:0), preferably a triglyceride, containing at least one caprylic acid (C8:0), or a
a triglyceride containing at least one short chain fatty acid C1-C5, preferably C2-C5, such as butyric acid (C4:0), and at least one medium chain fatty acid C6-C12, such as caprylic acid (C8:0), etc.

In an even more preferred aspect the lipid in the context of the composition are triglycerides comprising either butyric acid (C4:0) (BBB, Tributyrin) or caprylic acid (C8:0) (CCC, Tricaprylin), preferably a mix of triglycerides comprising either butyric acid (C4:0) or caprylic acid (C8:0), or may be selected from triglycerides containing both butyric acid (C4:0) and caprylic acid (C8:0) in the same triglyceride. The latter triglycerides may be prepared by inter-esterification of a mix of triglycerides comprising either butyric acid (C4:0) or caprylic acid (C8:0). Commercially available sources for BBB, Tributyrin (available e.g. by Sigma-Aldrich). Commercially available sources for CCC, Tricaprylin is e.g. Neobee 895 (available e.g. by Stepan Specialty).

Methods of preparing a triglyceride composed of a mixture of butyrate and long chain fatty acids are known in the art, for example, as described in WO2019228851, which is incorporated in its entirety by reference. Exemplary triglycerides comprising butyrate and a long chain fatty acid include 1,3-dibutyryl-2-linoleoylglycerol, 1,3-dibutyryl-2-stearoylglycerol, 1-butyryl-2-oleoyl-3-palmitoylglycerol, 1-palmitoyl-2-oleoyl-3-butyrylglycerol, 1-butyryl-2-oleoyl-3-linoleoylglycerol, 1-linoleoyl-2-oleoyl-3-butyrylglycerol, 1-oleoyl-2-butyryl-3-linoleoylglycerol, 1-linoleoyl-2-butyryl-3-oleoylglycerol, 1-butyryl-2-linoleoyl-3-oleoylglycerol, 1-oleoyl-2-linoleoyl-3-butyrylglycerol, 1-butyryl-2-stearoyl-3-oleoylglycerol, 1-oleoyl-2-stearoyl-3-butyrylglycerol, 1-butyryl-2-oleoyl-3-stearoylglycerol, and/or 1-stearoyl-2-oleoyl-3-butyrylglycerol, and mixtures of two or more thereof. A preferred long chain fatty acid in the context of a triglyceride comprising a mixture of butyrate and a long chain fatty acid is oleic acid (C18:1). A preferred triglyceride composed of a mixture of short and long chain fatty acids is a triglyceride consisting of a mixture of butyrate and oleate.

In the context of the composition are preferably triglycerides comprising fatty acids that could be metabolized into ketone bodies, preferably C4 and/or C8 short and medium chain fatty acids, more preferably triglycerides comprising both butyric acid (C4:0) and caprylic acid (C8:0) in the same triglyceride; or triglycerides comprising either butyric acid (C4:0) or caprylic acid (C8:0) in the same triglyceride; or a mix of triglycerides, one triglyceride comprising butyric acid (C4:0) and one triglyceride comprising caprylic acid (C8:0).

Alternatively, but less preferred, the lipid may also be selected from short chain fatty acids (SCFA) and medium chain fatty acids (MCFA), not a triglyceride. Such a lipid may therefore be selected from at least one short chain fatty acid C1-C5, preferably C2-C5, more preferably butyric acid (C4:0), or may be selected from at least one medium chain fatty acid C6-C12, such as caprylic acid (C8:0), or may be selected from a mixture of at least one short chain fatty acid C1-C5, preferably C2-C5, such as butyric acid (C4:0), and at least one medium chain fatty acid C6-C12, such as caprylic acid (C8:0).

According to a further aspect, the lipids are preferably contained in the composition as defined herein in an amount of between 0.1 g to 30 g per daily dose, preferably in an amount of between 2 g to 15 g per daily dose.

According to a preferred aspect, the composition as defined herein may comprise:
(a) a probiotic bacteria, preferably selected from probiotic bacteria,
   lacking a gene for producing at least one of urea, uric acid, p-cresol, p-cresyl sulfate, indoxyl sulfate, indole, indole acetic acid, indoxyl, trimethylamine, TMAO, dimethylglycine, betaine and/or glutarate;
   lacking at least one bacterial enzyme selected from urease, carnitine monooxygenase & reductase, tryptophanase and/or hydroxyphenylacetate; and/or
   expressing at least one bacterial enzyme selected from α-galactosidase, β-galactosidase, glucan 1,4-β-glucosidase, cellulase, β-fructofuranosidase and/or Licheninase;
(b) a prebiotic selected from carbohydrates, wherein the carbohydrate can be hydrolyzed by at least one bacterial enzyme selected from α-galactosidase, β-galactosidase, glucan 1,4-β-glucosidase, cellulase β-fructofuranosidase and/or Licheninase, preferably selected from EC3.2.1.22, EC3.2.1.23, EC3.2.1.74, EC 3.2.1.4, EC 3.2.1.26, EC 3.2.1.58, EC 3.2.1.73, EC 2.4.1.9, EC 3.2.1.84, EC 3.2.1.33, EC 3.2.1.70, EC 3.2.1.135, EC 3.2.1.3, EC 3.2.1.20, EC 3.2.1.10, or preferably wherein the carbohydrate is selected from the family of α-galacto-oligosaccharides/raffinose (e.g. pea GOS, soy GOS), β-galacto-oligosaccharides (e.g. b-GOS, Vivinal GOS, bovine milk oligosaccharides (BMOS)), human milk oligosaccharides (HMOs) (e.g. 2′-fucosyllactose (2′FL), 3-fucosyllactose (3′FL), lacto-N-neotetraose (LNnT), lacto-N-tetraose (LNT), 3′-sialyllactose (3SL), and/or 6′-sialyllactose (6SL)), cello-oligosaccharides (COS) (e.g. cellobiose, cellotriose, cellotetraose, soluble hydrolyzed wheat soluble hydrolyzed oat and/or β-glucan hydrolysates containing cellobiose or cellotriose or cellotetraose), or a combination thereof;
(c) a lipid, selected from a triglyceride, a short chain fatty acid and/or a medium chain fatty acid, wherein the triglyceride comprises short chain fatty acids, medium chain fatty acids, or a mixture thereof, further wherein in one aspect the triglyceride comprises butyrate and/or caprylate.

According to a more preferred aspect, the composition as defined herein may comprise a probiotic bacterium, a prebiotic and a lipid as defined above, wherein:
(a) the probiotic bacterium is selected from at least one of the probiotic bacteria according to a. to k. as defined below:
   a. Bifidobacterium animalis subspecies lactis NCC 2818 (CNCM I-3446);
   b. Bifidobacterium longum subspecies infantis NCC 341 (ATCC 15697 (T));
   c. Bifidobacterium longum subspecies longum NCC 2705 (CNCM I-2618) (NCBI refseq; GCA_000007525.1);
   d. Enterococcus faecium NCC 2768 (NCIMB 10415);
   e. Lactobacillus johnsonii NCC 533 (CNCM I-1225) (NCBI refseq; GCA_000008065.1);
   f. Lactococcus lactis NCC 2287 (CNCM I-4154);
   g. Lacticaseibacillus paracasei NCC 2461 (CNCM I-2116);
   h. Lacticaseibacillus rhamnosus NCC 4007 (CGMCC 1.3724);
   i. Staphylococcus carnosus NCC 1052 (CNCM I-5400);
   j. Staphylococcus carnosus NCC 971 (CNCM I-5398); and/or;
   k. Streptococcus thermophilus NCC 2496 (CNCM I-3915);
      or a probiotic bacterium having a genome that has at least 95% ANI, preferably at least 96% ANI, more preferably at least 97% ANI, even more preferably at least 98% ANI, most preferably at least 99% ANI or even 99.5% ANI or 99.9% ANI to any one of the genomic sequences of the probiotic bacteria according to a. to k. as defined above; or a combination of one or more of the above described probiotic bacterium;
(b) the prebiotic is selected from the family of α-galacto-oligosaccharides/raffinose (e.g. pea GOS, soy GOS), β-galacto-oligosaccharides (e.g. β-GOS, Vivinal GOS, bovine milk oligosaccharides), cello-oligosaccharides (COS) (e.g. cellobiose, cellotriose, cellotetraose, soluble hydrolyzed wheat, soluble hydrolyzed oat and/or β-glucan hydrolysates containing cellobiose or cellotriose or cellotetraose), human milk oligosaccharides (HMOs) (e.g. 2′-fucosyllactose (2′FL), 3-fucosyllactose (3′FL), lacto-N-neotetraose (LNnT), lacto-N-tetraose (LNT), 3′-sialyllactose (3SL), and/or 6′-sialyllactose (6SL)) or a combination thereof,
(c) the lipid is selected from a triglyceride, a short chain fatty acid and/or a medium chain fatty acid, wherein the triglyceride comprises short chain fatty acids, medium chain fatty acids, or a mixture thereof, further wherein in one aspect the triglyceride comprises butyrate and/or caprylate. Preferably the lipid is selected from:
   a. Triglycerides (TG) composed of a mixture of butyrate and medium chain fatty acids (MCFAs);
   b. Triglycerides (TG) composed of a mixture of short and medium chain fatty acids (SMCFAs);
   c. Triglycerides (TG) composed of a mixture of butyrate and long chain fatty acids (such as a Triglyceride composed of a mixture of butyrate and oleate);
   d. Triglycerides composed of a medium chain fatty acid;
   e. Triglycerides (TG) composed of a short chain fatty acid;
   f. Short chain fatty acids that could be metabolized into ketone bodies; and/or
   g. Short and medium chain fatty acids that could be metabolized into ketone bodies, preferably C4 and/or C8 short and medium chain fatty acids (such as butyric acid (C4:0) and/or caprylic acid (C8:0)).

According to an even more preferred aspect, the composition as defined herein may comprise a probiotic, a prebiotic and a lipid as defined above, wherein:
(a) the probiotic bacterium is selected from Lactobacillus johnsonii NCC 533 (CNCM I-1225) or a probiotic having a genome that has at least 95% ANI, preferably at least 96% ANI, more preferably at least 97% ANI, even more preferably at least 98% ANI, most preferably at least 99% ANI or even 99.5% ANI or 99.9% ANI to the genomic sequence of Lactobacillus johnsonii NCC 533 (CNCM I-1225) (SEQ ID NO: 1).
(b) the prebiotic is selected from the family of α-galacto-oligosaccharides/raffinose (e.g. pea GOS, soy GOS), β-galacto-oligosaccharides (e.g. β-GOS, Vivinal GOS, bovine milk oligosaccharides), cello-oligosaccharides (COS) (e.g. cellobiose, cellotriose, cellotetraose, soluble hydrolyzed wheat, soluble hydrolyzed oat and/or β-glucan hydrolysates containing cellobiose or cellotriose or cellotetraose), or a combination thereof;
(c) the lipid is selected from a triglyceride, a short chain fatty acid and/or a medium chain fatty acid, wherein the triglyceride comprises short chain fatty acids, medium chain fatty acids, or a mixture thereof, further wherein in one aspect the triglyceride comprises butyrate and/or caprylate. More preferably the lipid is selected from:
   a. Triglycerides (TG) composed of a mixture of butyrate and medium chain fatty acids (MCFAs);
   b. Triglycerides (TG) composed of a mixture of short and medium chain fatty acids (SMCFAs);
   c. Triglycerides (TG) composed of a mixture of butyrate and long chain fatty acids (such as a Triglyceride composed of a mixture of butyrate and oleate);
   d. Triglycerides composed of a medium chain fatty acid;
   e. Triglycerides (TG) composed of a short chain fatty acid;
   f. Short chain fatty acids that could be metabolized into ketone bodies; and
   g. Short and medium chain fatty acids that could be metabolized into ketone bodies, preferably C4 and/or C8 short and medium chain fatty acids.

According to an even further preferred aspect, the composition as defined herein may comprise a probiotic, a prebiotic and a lipid as defined above, wherein:
(a) the probiotic bacterium is selected from Lactobacillus johnsonii NCC 533 (CNCM I-1225) or a probiotic having a genome that has at least 95% ANI, preferably at least 96% ANI, more preferably at least 97% ANI, even more preferably at least 98% ANI, most preferably at least 99% ANI or even 99.5% ANI or 99.9% ANI to the genomic sequence of Lactobacillus johnsonii NCC 533 (CNCM I-1225) (SEQ ID NO: 1).
(b) the prebiotic is selected from pea GOS, soy GOS, β-GOS, Vivinal GOS, bovine milk oligosaccharides, human milk oligosaccharides, cellobiose, cellotriose, cellotetraose, soluble hydrolyzed wheat, soluble hydrolyzed oat and/or β-glucan hydrolysates containing cellobiose or cellotriose or cellotetraose, or a combination thereof;
(c) the lipid is selected from:
   a. Triglycerides (TG) composed of a mixture of butyrate and medium chain fatty acids (MCFAs);
   b. Triglycerides (TG) composed of a mixture of short and medium chain fatty acids (SMCFAs);
   c. Triglycerides (TG) composed of a mixture of butyrate and long chain fatty acids (such as a Triglyceride composed of a mixture of butyrate and oleate);
   d. Triglycerides composed of a medium chain fatty acid;
   e. Triglycerides (TG) composed of a short chain fatty acid;
   f. Short chain fatty acids that could be metabolized into ketone bodies; and
   g. Short and medium chain fatty acids that could be metabolized into ketone bodies, preferably C4 and/or C8 short and medium chain fatty acids (such as butyric acid (C4:0) and/or caprylic acid (C8:0)).

According to another aspect, the composition as defined herein is selected from a food product or nutritional composition, dietary supplements, a food for special medical purposes (FSMP), a nutritional supplement, a dairy-based drink, a low-volume liquid supplement, functional food products, functional beverage products, a meal replacement beverage, and combinations thereof. Additionally, without being limiting thereto, the composition may be in an administrable form, preferably selected from a pharmaceutical formulation.

In this context, it is particularly preferred that the composition may be solid or liquid, may be present in form of a powder, a tablet, a capsule, or may be in form of an oil formulation, an emulsion, an oil-in-water emulsion (o/w emulsion), or a water-in oil emulsion (w/o emulsion).

Each of the compounds of the composition can be administered at the same time as the other compounds (for example, as a single unit) or separated by a time interval (for example, in separate units). Preferably, the compounds are provided in form of a single unit.

The present invention particularly provides a nutrition-based solution for management of uremic toxins related to disease conditions and associated complications. The general application of such compositions can be in the form of a medical food or FSMP (food for special medical purposes) supplement, as a component of medical nutrition product, as an adjunct/concurrent to standard of care, as an adjunct/concurrent to kidney disease medication such as SGLT inhibitor, as an adjunct/concurrent to nutritional ingredients targeting kidney mitochondrial dysfunction, as an adjunct/concurrent to a protein diet, as a nutritional supplement, as a dairy-based drink, as a low-volume liquid supplement, as a meal replacement beverage, and combinations thereof, etc.

According to a second embodiment, the invention is directed to the use of the composition as defined herein for lowering or avoiding accumulation of uremic toxins, preferably in the treatment of cardiometabolic or neurodegenerative conditions or for preventing accumulation of uremic toxins, preferably in the treatment of cardiometabolic or neurodegenerative conditions. The composition is thereby preferably used for the reduction of uremic toxins in cardiometabolic or neurodegenerative conditions and related comorbidities, for delaying the progression of such cardiometabolic or neurodegenerative conditions and comorbidities and/or for managing symptoms and syndrome associated with toxic effects of uremic solutes of such cardiometabolic or neurodegenerative conditions and related comorbidities, and/or for preventing accumulation of uremic toxins in the treatment of cardiometabolic or neurodegenerative conditions and related comorbidities.

Cardiometabolic or neurodegenerative conditions and related comorbidities that can be treated by using the composition preferably include the following cases:
treatment or prevention of Kidney disease, including chronic and acute; dialysis and pre-dialysis states of kidney disease; rare (kidney) diseases, genetic- and metabolic-induced (kidney) diseases;
treatment or prevention of Uremic syndrome, including protein energy wasting syndrome, bone-loss, hyper anorexia, fatigue, or inflammation;
delay of advanced kidney disease complication, including anemia, hyperphosphatemia, secondary hyperparathyroidism, bone disease, and neurological conditions;
delay of kidney disease comorbidities, including cardiovascular disease;
prevention of the risk or management of malnutrition;
delay of the progression of cardiometabolic disease;
prevention of the risk or management of cardiovascular disease and comorbidities, such as diabetes; and/or
prevention of the risk or management of neurodegenerative and neurological conditions.

Generally, subject for any such treatments or a patient in need of such a treatment is a mammal, preferably, a human or a pet, such as a companion animal, suffering from any of the above mentioned cardiometabolic or neurodegenerative conditions and related comorbidities. Preferably, a subject for any such treatments or a patient in need of such a treatment may be a child, a toddler or an infant, an elderly, a companion animal, but also companion pets, such as a cat or a dog, wherein the subject is preferably either at risk of developing such a disease or has already developed such a disease.

According to a third embodiment, provided is a method of treatment of cardiometabolic or neurodegenerative conditions as defined above, preferably comprising as a first step (a) a step of preparing and providing a composition as discussed above comprising the specifically selected probiotic, the specifically selected prebiotic and the specifically selected lipid as defined above; and (b) administering such a composition to a patient in need thereof, typically suffering from an increase of uremic toxins, typically in the context of cardiometabolic or neurodegenerative conditions as defined herein.

According to a fourth embodiment, provided is a kit (of parts) suitable for use in lowering and/or avoiding accumulation of uremic toxins in cardiometabolic or neurodegenerative conditions as defined herein comprising the composition as described herein, comprising the specifically selected probiotic, bacterium, the specifically selected prebiotic and the specifically selected lipid as defined above; for admixing to form one or more of the compositions disclosed herein and/or for use in one or more of the methods disclosed herein, for example in separate containers as two or more liquid solutions or dried powders. The kit may also include instructions for use.

It should be appreciated that the various aspects and embodiments of the detailed description as disclosed herein are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

Preferred features and embodiments of the invention will now be described by way of non-limiting examples.

The invention includes the following embodiments:

### Embodiment 1

Composition for use in lowering or avoiding accumulation of uremic toxins in cardiometabolic or neurodegenerative conditions, wherein the composition comprises:
(a) a probiotic bacterium, selected from probiotic bacteria
   lacking a gene for producing at least one of urea, uric acid, p-cresol, p-cresyl sulfate, indoxyl sulfate, indole, indole acetic acid, indoxyl, trimethylamine, TMAO, dimethylglycine, PCG, CMPF, betaine and/or glutarate;
   lacking at least one bacterial enzyme selected from urease, carnitine monooxygenase & reductase, tryptophanase and/or hydroxyphenylacetate; and/or
   expressing at least one bacterial enzyme selected from α-galactosidase, β-galactosidase, glucan 1,4-β-glucosidase, cellulase, β-fructofuranosidase and/or Licheninase;
(b) a prebiotic selected from carbohydrates or fibers, wherein the carbohydrate or fiber can be hydrolyzed by at least one bacterial enzyme selected from α-galactosidase, β-galactosidase, glucan 1,4-β-glucosidase, cellulase, β-fructofuranosidase and/or Licheninase;
(c) a lipid, selected from a triglyceride, a short chain fatty acid and/or a medium chain fatty acid, wherein the triglyceride comprises short chain fatty acids, medium chain fatty acids, long chain fatty acids, or a mixture thereof, further wherein in one aspect the triglyceride comprises butyrate and/or caprylate, and in one aspect the triglyceride consists of butyrate and caprylate or butyrate and oleate, further wherein preferably the triglyceride consists of butyrate and caprylate.

### Embodiment 2

The composition for use according to Embodiment 1, wherein the probiotic bacterium (a) is selected from at least one of the following a. to k.:
a. Bifidobacterium animalis subspecies lactis NCC 2818 (CNCM I-3446);
b. Bifidobacterium longum subspecies infantis NCC 341 (ATCC 15697 (T));
c. Bifidobacterium longum subspecies longum NCC 2705 (CNCM I-2618) (NCBI refseq; GCA_000007525.1);
d. Enterococcus faecium NCC 2768 (NCIMB 10415);
e. Lactobacillus johnsonii NCC 533 (CNCM I-1225) (NCBI refseq; GCA_000008065.1);
f. Lactococcus lactis NCC 2287 (CNCM I-4154);
g. Lacticaseibacillus paracasei NCC 2461 (CNCM I-2116);
h. Lacticaseibacillus rhamnosus NCC 4007 (CGMCC 1.3724);
i. Staphylococcus carnosus NCC 1052 (CNCM I-5400);
j. Staphylococcus carnosus NCC 971 (CNCM I-5398); and/or;
k. Streptococcus thermophilus NCC 2496 (CNCM I-3915);
or a probiotic having a genome that has at least 95% Average Nucleotide Identity (ANI), preferably at least 96% ANI, more preferably at least 97% ANI, even more preferably at least 98% ANI, most preferably at least 99% ANI or even 99.5% ANI or 99.9% ANI to any one of the genomic sequences of the probiotic bacteria according to any one of a. to k. as defined above.

### Embodiment 3

The composition for use according to Embodiment 2, wherein the composition comprises two or more probiotic bacteria defined in a. to k.

### Embodiment 4

The composition for use according to Embodiment 2, wherein the composition comprises two or more probiotic bacteria having a genome that has at least 99% ANI to the genomic sequence of the probiotic bacteria according to a. to k.

### Embodiment 5

The composition for use according to Embodiment 2, wherein the probiotic bacterium (a) is Lactobacillus johnsonii NCC 533 (CNCM I-1225) (NCBI refseq; GCA_000008065.1) or a probiotic bacterium having a genome that has at least 95% ANI, preferably at least 96% ANI, more preferably at least 97% ANI, even more preferably at least 98% ANI, most preferably at least 99% ANI or even 99.5% ANI or 99.9% ANI to the genomic sequence of Lactobacillus johnsonii NCC 533 (CNCM I-1225) (NCBI refseq; GCA_000008065.1, SEQ ID NO: 1).

### Embodiment 6

The composition for use according to any one of Embodiments 1 to 5, wherein the prebiotic (b) is selected from α-galacto-oligosaccharides (α-GOS)/raffinose, β-galacto-oligosaccharides (β-GOS), and cello-oligosaccharides (COS), human milk oligosaccharides (HMOs), or a combination thereof.

### Embodiment 7

The composition for use according to any one of Embodiments 1 to 6, wherein the prebiotic (b) is selected from soy GOS (α-GOS), pea GOS (α-GOS), bovine milk oligosaccharides (BMOS) (β-GOS), Vivinal GOS (β-GOS), human milk oligosaccharides (HMOs) (2′-fucosyllactose (2′FL), 3-fucosyllactose (3′FL), lacto-N-neotetraose (LNnT), lacto-N-tetraose (LNT), 3′-sialyllactose (3 SL), and/or 6′-sialyllactose (6SL)), Cellobiose, Cellotriose, Cellotetraose, and soluble hydrolyzed wheat, soluble hydrolyzed oat and/or β-glucan hydrolysates containing cellobiose or cellotriose or cellotetraose, or a combination thereof.

### Embodiment 8

The composition for use according to any one of Embodiments 1 to 7, wherein the lipid (c) is selected from:
a. Triglycerides (TG) composed of a mixture of butyrate (C4:0) and medium chain fatty acids (MCFAs);
b. Triglycerides (TG) composed of a mixture of short and medium chain fatty acids (SMCFAs), preferably C4 and/or C8 fatty acids;
c. Triglycerides (TG) composed of a mixture of butyrate and long chain fatty acids (such as a Triglyceride composed of a mixture of butyrate and oleate);
d. Triglycerides composed of a medium chain fatty acid;
e. Triglycerides (TG) composed of a short chain fatty acid;
f. Short chain fatty acids that could be metabolized into ketone bodies; and
g. Short and medium chain fatty acids that could be metabolized into ketone bodies, preferably C4 and/or C8 fatty acids (such as butyric acid (C4:0) and/or caprylic acid (C8:0)).

### Embodiment 9

The composition for use according to any of Embodiments 1 to 8, wherein:
a. the probiotic bacterium is selected from at least one of the probiotic bacteria according to a. to k. as defined above, or a probiotic having a genome that has at least 90% ANI, preferably at least 95% ANI to any of the genomes of the probiotic bacteria according to a. to k. as defined above, or a combination thereof, preferably from Lactobacillus johnsonii NCC 533 (CNCM I-1225) or a probiotic having a genome that has at least 90% ANI, preferably at least 95% ANI to the genomic sequence of Lactobacillus johnsonii NCC 533 (CNCM I-1225);
b. the prebiotic is selected from α-galacto-oligosaccharides/raffinose, β-galacto-oligosaccharides, or cello-oligosaccharides, or a combination thereof, preferably selected from soy GOS (α-GOS), pea GOS (α-GOS), bovine milk oligosaccharides (BMOS) (β-GOS), Vivinal GOS (β-GOS), cellobiose, cellotriose, cellotetraose, soluble hydrolyzed wheat, soluble hydrolyzed oat and/or β-glucan hydrolysates containing cellobiose or cellotriose or cellotetraose, or a combination thereof,
c. the lipid is selected from a triglyceride, a short chain fatty acid and/or a medium chain fatty acid, wherein the triglyceride comprises short chain fatty acids, medium chain fatty acids, or a mixture thereof, further wherein in one aspect the triglyceride comprises butyrate and/or caprylate, preferably from
   a. Triglycerides (TG) composed of a mixture of butyrate and medium chain fatty acids (MCFAs);
   b. Triglycerides (TG) composed of a mixture of short and medium chain fatty acids (SMCFAs), preferably C4 and/or C8 short and medium chain fatty acids;
   c. Triglycerides (TG) composed of a mixture of butyrate and long chain fatty acids (such as a Triglyceride composed of a mixture of butyrate and oleate);
   d. Triglycerides composed of a medium chain fatty acid;
   e. Triglycerides (TG) composed of a short chain fatty acid;
   f. Short chain fatty acids that could be metabolized into ketone bodies; and
   g. Short and medium chain fatty acids that could be metabolized into ketone bodies, preferably C4 and/or C8 short and medium chain fatty acids (such as butyric acid (C4:0) and/or caprylic acid (C8:0)).

### Embodiment 10

The composition for use according to embodiment 9, wherein the probiotic bacterium (a) is selected from Lactobacillus johnsonii NCC 533 (CNCM I-1225) (NCBI refseq; GCA_000008065.1) or a probiotic having a genome that has at least 90% ANI, preferably at least 95% ANI to the genomic sequence of Lactobacillus johnsonii NCC 533 (CNCM I-1225) (NCBI refseq; GCA_000008065.1).

### Embodiment 11

The composition for use according to Embodiment 7 or Embodiment 8, wherein the prebiotic (b) selected from soy GOS (α-GOS), pea GOS (α-GOS), bovine milk oligosaccharides (BMOS) (β-GOS), Vivinal GOS (β-GOS), cellobiose, cellotriose, cellotetraose, soluble hydrolyzed wheat, soluble hydrolyzed oat and/or β-glucan hydrolysates containing cellobiose or cellotriose or cellotetraose, or a combination thereof.

### Embodiment 12

The composition for use according to any one of Embodiments 9 to 11, wherein the lipid (c) is selected from:
a. Triglycerides (TG) composed of a mixture of butyrate and medium chain fatty acids (MCFAs);
b. Triglycerides (TG) composed of a mixture of short and medium chain fatty acids (SMCFAs);
c. Triglycerides (TG) composed of a mixture of butyrate and long chain fatty acids (such as a Triglyceride composed of a mixture of butyrate and oleate);
d. Triglycerides composed of a medium chain fatty acid;
e. Triglycerides (TG) composed of a short chain fatty acid;
f. Short chain fatty acids that could be metabolized into ketone bodies; and
g. Medium chain fatty acids that could be metabolized into ketone bodies, preferably C4 and/or C8 medium chain fatty acids (such as butyric acid (C4:0) and/or caprylic acid (C8:0));

### Embodiment 13

The composition for use according to any one of Embodiment 1 to 12, wherein the probiotic bacteria are contained in an amount of between 10³ cfu to 10¹² cfu per daily dose, typically in an amount of between 10⁴ cfu to 10¹¹ cfu per daily dose, preferably in an amount of between 10⁵ cfu to 10¹⁰ cfu per daily dose, or 10⁵ cfu to 10⁹ cfu per daily dose, likewise preferably in an amount of between 10⁶ cfu to 10⁹ cfu per daily dose, 10⁶ cfu to 10⁸ cfu per daily dose or in an amount of 10⁸ cfu to 10¹⁰ cfu per daily dose, more preferably around 10⁷ cfu to 10⁹ cfu per daily dose.

### Embodiment 14

The composition for use according to one any of Embodiment 1 to 13, wherein the prebiotics are contained in an amount of 0.1 g to 30 g per daily dose, preferably in an amount of between 2 g to 15 g per daily dose.

### Embodiment 15

The composition for use according to any one of Embodiments 1 to 14, wherein the lipids are contained in an amount of 0.1 g to 30 g per daily dose, preferably in an amount of between 2 g to 15 g per daily dose.

### Embodiment 16

The composition for use according to any one of Embodiments 1 to 15, wherein the lipid is a triglyceride consisting of butyrate and caprylate or a triglyceride consisting of butyrate and oleate.

### Embodiment 17

The composition for use according to any one of Embodiments 1 to 15, wherein the lipid is a triglyceride consisting of butyrate and caprylate.

### Embodiment 18

The composition for use according to any one of Embodiments 1 to 17, which is in form of a food product or nutritional composition, dietary supplement, a food for special medical purposes (FSMP), a nutritional supplement, a dairy-based drink, a low-volume liquid supplement, functional food product, functional beverage product, a meal replacement beverage, and combinations thereof.

### Embodiment 19

The composition for use according to any one of Embodiments 1 to 18, wherein the composition is provided in form of a powder, a tablet, a capsule, or may be in form of an oil formulation, an emulsion, an oil-in-water emulsion (o/w emulsion), or a water-in oil emulsion (w/o emulsion).

### Embodiment 20

The composition for use according to any one of Embodiments 1 to 19, wherein the reduction of uremic toxins in cardiometabolic or neurodegenerative conditions is for delaying the progression of such diseases and comorbidities and/or for managing symptoms and syndrome associated with toxic effects of uremic solutes of such diseases and comorbidities.

### Embodiment 21

The composition for use according to any one of Embodiments 1 to 19, wherein the reduction of uremic toxins in cardiometabolic or neurodegenerative conditions includes:
treatment or prevention of Kidney disease, including chronic and acute; dialysis and pre-dialysis; rare diseases, genetic- and metabolic-induced;
treatment or prevention of Uremic syndrome, including protein energy wasting, bone-loss, hyper anorexia, fatigue, or inflammation;
delay of advanced kidney disease complication, including anemia, hyperphosphatemia, secondary hyperparathyroidism, bone disease, neurological conditions;
delay of kidney disease comorbidities, including cardiovascular disease;
prevention of the risk or management of malnutrition;
delay of the progression of cardiometabolic disease;
prevention of the risk or management of cardiovascular disease and comorbidities (diabetes); and/or
prevention of the risk or management of neurodegenerative and neurological conditions

### Embodiment 22

Kit of parts, suitable for use in lowering or avoiding accumulation of uremic toxins in cardiometabolic or neurodegenerative conditions, and comprising the composition as defined according to any one of Embodiments 1 to 17, comprising the probiotic, the prebiotic and the lipid in two or more separate containers, and optionally further comprising an instruction manual.

### Embodiment 23

Method of treatment of cardiometabolic or neurodegenerative conditions as defined above, comprising as a first step (a) a step of preparing and providing a composition according to any one of Embodiments 1 to 17, comprising the probiotic, the prebiotic and the lipid; and (b) administering such a composition to a patient in need thereof suffering from an increase of uremic toxins in the context of a cardiometabolic or neurodegenerative condition.

### Embodiment 24

Method of treating a cardiometabolic or neurodegenerative condition as defined in Embodiment 21, comprising administering to a patient a composition according to any one of Embodiments 1 to 17.

### Embodiment 25

Use of a composition defined in any one of Embodiments 1 to 17 in the manufacture of a medicament for the treatment of a cardiometabolic or neurodegenerative condition as defined in Embodiment 21.

### Embodiment 26

The composition for use according to any one of Embodiments 1 to 15 and 17 to 21, the kit of parts of Embodiment 22, the method of Embodiment 23 or Embodiment 24, or the use of Embodiment 25 wherein the composition comprises Lactobacillus johnsonii NCC533 10⁹, cellobiose 1%, short-medium-chain triglyceride containing butyrate and caprylate 1%.

### Embodiment 27

The composition for use according to any one of Embodiments 1 to 15 and 17 to 21, the kit of parts of Embodiment 22, the method of Embodiment 23 or Embodiment 24, or the use of Embodiment 25 wherein the composition comprises Lactobacillus johnsonii NCC533 10⁹, cellobiose 1%, pea GOS 1%, short-medium-chain triglyceride containing butyrate and caprylate 1%.

### Embodiment 28

The composition for use according to any one of Embodiments 1 to 19, 26 and 27, the kit of parts of Embodiment 20, 26 and 27, the method of any one of Embodiments 21, 22, 26 and 27, or the use of any one of Embodiments 23, 26 and 27, wherein the composition further comprises one or more HMOs, preferably one or more of 2′-fucosyllactose (2′FL), 3-fucosyllactose (3′FL), lacto-N-neotetraose (LNnT), lacto-N-tetraose (LNT), 3′-sialyllactose (3SL), or 6′-sialyllactose (6SL).

### Embodiment 29

The composition for use according to any one of Embodiments 1 to 19, 26 and 27, the kit of parts of Embodiment 20, 26 and 27, the method of any one of Embodiments 21, 22, 26 and 27, or the use of any one of Embodiments 23, 26 and 27, wherein the composition further comprises one or more of 2′-fucosyllactose (2′FL), 3-fucosyllactose (3′FL), lacto-N-neotetraose (LNnT), or lacto-N-tetraose (LNT).

### Embodiment 30

The composition for use, the kit of parts, the method, or the use of Embodiment 28 or Embodiment 29 further comprising one or more of
a. Bifidobacterium animalis subspecies lactis NCC 2818 (CNCM I-3446);
b. Bifidobacterium longum subspecies infantis NCC 341 (ATCC 15697 (T)); and
c. Bifidobacterium longum subspecies longum NCC 2705 (CNCM I-2618) (NCBI refseq; GCA_000007525.1).

### Embodiment 31

The composition for use according to any one of Embodiments 1 to 15 and 18 to 21, the kit of parts of Embodiment 20, 26 and 27, the method of any one of Embodiments 21, 22, 26 and 27, or the use of any one of Embodiments 23, 26 and 27, wherein the composition comprises a triglyceride comprised of butyrate and a long chain fatty acid.

### Embodiment 32

The composition for use, the kit of parts, the method, or the use of Embodiment 31, wherein the triglyceride comprised of butyrate and a long chain fatty acid is one or more of triglycerides comprising butyrate and a long chain fatty acid include 1,3-dibutyryl-2-linoleoylglycerol, 1,3-dibutyryl-2-stearoylglycerol, 1-butyryl-2-oleoyl-3-palmitoylglycerol, 1-palmitoyl-2-oleoyl-3-butyrylglycerol, 1-butyryl-2-oleoyl-3-linoleoylglycerol, 1-linoleoyl-2-oleoyl-3-butyrylglycerol, 1-oleoyl-2-butyryl-3-linoleoylglycerol, 1-linoleoyl-2-butyryl-3-oleoylglycerol, 1-butyryl-2-linoleoyl-3-oleoylglycerol, 1-oleoyl-2-linoleoyl-3-butyrylglycerol, 1-butyryl-2-stearoyl-3-oleoylglycerol, 1-oleoyl-2-stearoyl-3-butyrylglycerol, 1-butyryl-2-oleoyl-3-stearoylglycerol, and/or 1-stearoyl-2-oleoyl-3-butyrylglycerol, and mixtures of two or more thereof.

### Embodiment 33

The composition for use, the kit of parts, the method, or the use of Embodiment 31, wherein the triglyceride comprised of butyrate and a long chain fatty acid is a mixture of butyrate and a long chain fatty acid is oleic acid (C18:1).

### Embodiment 34

The composition for use, the kit of parts, the method, or the use of any one of Embodiments 31 to 33, wherein the composition further comprises one or more HMOs, preferably one or more of 2′-fucosyllactose (2′FL), 3-fucosyllactose (3′FL), lacto-N-neotetraose (LNnT), lacto-N-tetraose (LNT), 3′-sialyllactose (3SL), or 6′-sialyllactose (6SL).

### Embodiment 35

The composition for use, the kit of parts, the method, or the use of any one of Embodiments 31 to 33, wherein the composition further comprises one or more of 2′-fucosyllactose (2′FL), 3-fucosyllactose (3′FL), lacto-N-neotetraose (LNnT), or lacto-N-tetraose (LNT).

### EXAMPLES

### Method 1: Ingredient Selection

In Silico Selection of Probiotic Stains with Low Capacity to Produce Clinically Relevant Uremic Toxins

The Nestle inhouse bacterial genomics platform WallGene (containing all genomes of Nestle Culture Collection) complemented with internal BlastP (Basic Local Alignment Search Tool for protein sequences, (Altschul et al., 1990)) searches were used to select probiotic strains from NCC with low capacity to contribute to uremic toxins production.

Specifically, a Blast database containing all predicted proteins of the probiotic strains was created. Bacterial and host metabolic pathways involved in uremic toxin metabolism were compiled from public database like KEGG pathway (Kyoto Encyclopedia of Genes and Genomes) and literature. The amino acid sequences of the key enzymes involved in uremic toxin metabolism were further extracted from public database like KEGG, UniProt and SwissProt according to the EC number, and complied in a FASTA file (list of reference protein sequences). These FASTA files, containing the protein sequences of key enzymes, were used as the reference sequences to perform BLASTp against the protein profiles of available probiotic strains from NCC. When pBLAST showed a low similarity to the reference protein (e.g. identity <30%), InterProScan (EMBL-EBI) was further used to predict the presence of functional domains (Quevillon et al., 2005).

In Silico Selection of Carbohydrate Substrates Able to Support the Growth of Selected Probiotic Strains with Low Uremic Toxins Production Capacity

The Carbohydrate Active enZymes (CAZy) (Lombard et al., 2014) of the candidate probiotics were annotated in silico using dbCAN (Yin et al., 2012). Based on the probiotic CAZy annotation, different carbohydrates displaying the appropriate biochemical structures are selected using BRENDA—The Comprehensive Enzyme Information System. The list of substrates per CAZy are subsequently matched with the carbohydrate ingredients constituted by poly- and/or oligosaccharide structures corresponding to those of the preferred substrates.

### In Vitro Growth Tests to Validate in Silico Probiotic and Carbohydrate Candidate Combinations

The candidate probiotics are obtained from Nestle Culture Collection (NCC) or from publicly available deposited strains and culture collections (ATCC, NCIMB, NCBI). The carbohydrate ingredients are sourced from commercial supplier or from Nestle or its subsidies. A complete list of exemplary carbohydrate sources could be found in Table 1. Table 1 shows exemplary different genes of Lactobacillus johnsonii NCC 533 with Carbohydrate-Active Enzymes (CAZymes), Enzyme Code (EC) numbers of functional enzymes, their functions, suitable and related carbohydrate families, recommended Fiber ingredients in view of inventive preclinical tests, and availability of such compounds. As can be seen, the carbohydrates were selected already in an in-silico process based on their biochemical structural characteristics to be fermented or metabolized or used or broken-down or degraded or converted by the Carbohydrate-Active Enzymes (CAZymes) present in the genome of Lactobacillus johnsonii NCC 533.

TABLE 1

| L. johnsonii | | | | | | |
|---|---|---|---|---|---|---|
| NCC533 | CAZy family | EC | | Carbohydrate biochemical | Candidate carbohydrates | Carbohydrate |
| gene ID | annotation | number | Function | family | ingredients | Sources/Availability |
| CDS0227 | GH36 | 3.2.1.22 | α-galactosidase | α-galacto-oligosaccharides | Pea oligosaccharides Soy | AlphaGOS ® (Olygose, |
| | | | | (raffinose family) | oligosaccharides | AIDP) |
| | | | | Galactomannans | Partially-hydrolyzed | Sunfiber ® (Olygose, |
| | | | | | guar gum (PHGG) | AIDP) |
| | | | | | Fenugreek | Canafen ® (Emerald |
| | | | | | | Seed Products Ltd.) |
| CDS0956 | GH42 | 3.2.1.23 | β-galactosidase | β-galacto-oligosaccharides | Bovine milk | Nestlé |
| CDS0959 | GH2 | | | | oligosaccharides (BMOs) | |
| CDS0960 | GH2 | | | | | |
| CDS0604 | GH42 | | | | β-galacto- | Vivinal ® GOS |
| CDS0819 | GH42 | | | | oligosaccharides | (Friesland Campina) |
| CDS0602 | GH1 | 3.2.1.74 | glucan 1,4-β- | Cellooligosaccharides | Cellulose 0-glucan | Nestlé (hydrolysates |
| | | | glucosidase | | hydrolysates (contains | lab-scale produced |
| | | | | | glucose, cellobiose, | from Vitacel ® oat or |
| | | | | | cellotriose, cellotetraose) | wheat fiber |
| | | | | | Soluble hydrolyzed oat | |
| | | | | | Soluble hydrolyzed | |
| | | | | | wheat | |
| CDS0691 | GH5 | 3.2.1.4 | Cellulase | Cellooligosaccharides | Cellulose 0-glucan | Nestlé (lab-scale |
| | | | | | hydrolysates (contains | produced) |
| | | | | | glucose, cellobiose, | |
| | | | | | cellotriose, cellotetraose) | |
| CDS1761 | GH32 | 3.2.1.26 | β- | a-galacto-oligosaccharides | Pea oligosaccharides Soy | AlphaGOS ® (Olygose, |
| | | | fructofuranosidase | (raffinose family) | oligosaccharides | AIDP) |
| | | | | Fructans | Short-chain Fructo- | Orafti ® P95 (Beneo) |
| | | | | | oligosaccharides (ScFOS) | |
| | | | | | Inulin Chicory root fiber | |
| CDS0107 | GH8 | 3.2.1.73 | Licheninase | Cellooligosaccharides | Cellobiose Cellotriose | Megazyme |
| | | | | | Cellotetraoise | |

In vitro growth test of probiotic and carbohydrate candidates are performed with BioLector™ (m2p-labs, Baesweiler, Germany). Each run is tested in a 48-well flowerplate (m2p-labs, Baesweiler, Germany) with 1 mL volume per well. The strain is cultured in an MRS media without sugar (MRSapi) to which the different carbohydrates is added (final concentration 1%). Plates are grown anaerobically with a CO2 gas phase shaking at 300 rpm for 48 h. All cultures are inoculated using 2% of a fresh overnight culture. Biomass as measured by optical density (OD) at gain 30 and pH changes of each well are recorded over the incubation period. After the run, all data are collectively provided in excel sheet for further analysis.

### Preparation of the Ingredient Combinations for Pre-Clinical and Clinical Trials

Probiotic and lipid ingredients are supplied by Nestle or its subsidies (e.g. Sofinol SA, Konolfingen Nestle Factory). Carbohydrate ingredients are sourced from a variety of food-grade suppliers, such as Olygose (France), AIDP Inc (USA) and Savanna Ingredients GmbH (Germany). Microbiome ingredient combinations are produced through multi-step procedures:

Step 1: Encapsulation of lipid oil ingredients: BiPro is hydrated into water under stirring (Ystram) and lipid oils are slowly added (Polytron). The resulting mixture is heated under stirring at 82 °C for 10 min to fix the encapsulation.

Step 2: Spray drying of lipid-prebiotic ingredients: prebiotics are suspended in water and mixed with lipid suspensions. The mixtures are then homogenized (Ystram), pasteurized at 72 °C 2 min, and then spray dried at 140 °C. The final product is a white powder.

Step 3: Addition of probiotics: probiotics are added to the spray-dried prebiotic-lipid powder mixture. Turbula are then used to ensure a homogenous repartition of the probiotics into the powder.

### Method 2: In-Vitro/Ex-Vivo Investigation of the Relevance of the Microbiome Ingredients in Improving Uremic Toxins Generation

### Preservation of Fecal Samples

Fecal material was collected from eight CKD and nine healthy adult donors. Fecal suspensions were prepared and mixed with ProDigest optimized cryoprotectant, i.e. a modified version of the cryoprotectant developed by Hoefman et al. (2013). The obtained suspensions were aliquoted, flash frozen and then preserved at −80 °C (cryostock). Just before the experiment, an aliquot was defrosted and immediately added to the reactors.

### Short-Term Colonic Experiment to Evaluate the Difference on Metabolic Profile Between the Microbiome of CKD and Healthy Individuals

The short-term colonic experiments were performed in a single reactor to investigate overall fermentative activity, such as saccharolytic and proteolytic activity, and changes in microbial metabolite production by the fecal microbiome from healthy and CKD individuals. The short-term colonic experiments represent a simplified simulation of the continuous Simulator of the Human Microbial Ecosystem (SHIME®, ProDigest).

At the start of the experiment, the amino acid mix consisting of L-tryptophan, L-tyrosine, L-carnitine, choline and L-phenylalanine was added with a sugar-depleted nutritional background medium (containing basal nutrients of the colon including peptone, yeast extract, mucin and L-cystein) to the reactors. Then, 10% (v/v) of a cryostock containing 7.5% fecal inoculum of each of the investigated donors (which served as microbial source) was added, bringing the total volume in the reactors to 70 mL. A reference condition (or negative control) containing only the nutritional medium (without spiked AA) was included for each donor.

Reactors were incubated for 48 h at 37 °C, under continuous mild shaking (90 rpm) and anaerobic atmosphere. The incubations were performed in fully independent reactors with sufficiently high volume to not only ensure robust microbial fermentation, but also to enable the collection of multiple samples over time (0 h, 24 h, 48 h).

Samples at different time points were assessed for changes in pH, gas, short-chain fatty acids, ammonium, lactate, uremic toxins and precursors.

Adapted SHIME Setup to Assess the Impact of Microbiome Ingredients on Microbiome from CKD Patients

To optimally address the ability of the intervention to correct the microbiome dysbiosis in CKD patients, the Prodigest's SHIME® setup was adapted by combining the upper gastrointestinal tract with a single colonic region. The colonic region simulated the transverse colon (TC) conditions, having a pH of 6.2 till 6.6 and a retention time of 32 h. In this way, a diverse microbial community can be maintained in the system, being able to perform both saccharolytic as well as proteolytic fermentation processes. During this specific study example, the impact of two test products on the composition and metabolic activity of the microbiome of eight CKD donors was evaluated compared to a negative control for each of the donors tested, resulting in 24 different test conditions.

The SHIME® experiment for this study consisted of two stages:

(1) Inoculation period: On the first day, the colon reactors were inoculated with an appropriate fecal sample (conserved frozen as part of phase 1 of the study) and was allowed to grow and colonize the reactor. After this overnight incubation, the colon reactors were fed with the basic nutritional matrix for two more days, to support the maximum diversity of the gut microbiota originally present in the fecal inoculum. This also allowed the microbial community to differentiate in the different reactors depending on the local environmental conditions, while still retaining its CKD characteristics.

(2) Control/treatment period: During this 11-day period, the SHIME reactor was operated under nominal conditions and fed 3×/day with the SHIME nutritional medium. On the first day of this period (=d0), all arms were operated under nominal conditions and samples collected on this day provided the baseline parameters. From the second day of this period (=d1), the diet was supplemented with the test product in the treatment arms of each donor, while in the control arm of each donor the nominal conditions were maintained. On day 8 of this period, all arms of the SHIME were further supplemented with an amino acid mix. Samples were taken from the colon reactors throughout this period to investigate the specific effect of the test product on the resident microbial community composition and activity compared to the negative control.

During each period, the model was fed with SHIME nutritional medium 3 times per day. The feeding schedule is shown in the following table (Table 2).

TABLE 2

| | | Daily SHIME | Daily SHIME | Daily SHIME |
|---|---|---|---|---|
| | SHIME | Feed 1 (colonic | Feed 2 (colonic | Feed 3 (colonic |
| Period | arm | entrance at 9 h) | entrance at 17 h) | entrance at 1 h) |
| Inoculation | Control | Nominal SHIME | Nominal SHIME | Nominal SHIME |
| | | feed with NO | feed with NO | feed with NO |
| | | addition of test | addition of test | addition of test |
| | | product | product | product |
| | Treatment | Nominal SHIME | Nominal SHIME | Nominal SHIME |
| | | feed with NO | feed with NO | feed with NO |
| | | addition of test | addition of test | addition of test |
| | | product | product | product |
| Control / | Control | Nominal SHIME | Nominal SHIME | Nominal SHIME |
| Treatment | | feed with NO | feed with NO | feed with NO |
| period: d 0 | | addition of test | addition of test | addition of test |
| | | product | product | product |
| | Treatment | Nominal SHIME | Nominal SHIME | Nominal SHIME |
| | | feed with NO | feed with NO | feed with NO |
| | | addition of test | addition of test | addition of test |
| | | product | product | product |
| Control / | Control | Nominal SHIME | Nominal SHIME | Nominal SHIME |
| Treatment | | feed with NO | feed with NO | feed with NO |
| period: | | addition of test | addition of test | addition of test |
| d 1-d 7 | | product | product | product |
| | Treatment | Carbohydrate- | Carbohydrate- | Nominal SHIME |
| | | depleted SHIME | depleted SHIME | feed with NO |
| | | feed WITH | feed WITH | addition of test |
| | | addition of test | addition of test | product |
| | | product | product | |
| Control / | Control | Nominal SHIME | Nominal SHIME | Nominal SHIME |
| Treatment | | feed with NO | feed with NO | feed with NO |
| period: | | addition of test | addition of test | addition of test |
| d 8-d 10 | | product | product | product BUT with |
| | | | | addition of amino |
| | | | | acid mix |
| | Treatment | Carbohydrate- | Carbohydrate- | Nominal SHIME |
| | | depleted SHIME | depleted SHIME | feed with NO |
| | | feed WITH | feed WITH | addition of test |
| | | addition of test | addition of test | product BUT with |
| | | product | product | addition of amino |
| | | | | acid mix |

Samples at different time points (d0, d1, d2, d4, d7, d8, d10) were collected and assessed for changes in pH, gas, short-chain fatty acids, ammonium, lactate, uremic toxins and precursors.

### Analysis of the Microbial Community Composition and Activity

The large volumes in the colonic regions allow to collect sufficient volumes of liquids each day, without disturbing the microbial community or endangering the rest of the experiment. Several microbial parameters are monitored throughout the short-term colonic and SHIME experiments.

### (a) Overall Fermentative Activity:

pH: the degree of acidification during the experiment is a measure for the intensity of bacterial metabolism and is used as a process control parameter. The pH of the incubations provides a rough indication about the speed of fermentation of the test products.
Gas production: the incubations were performed in closed systems, which allows to measure accumulation of gases in the headspace with a pressure meter. Gas production is a measure of microbial activity, and thus of the speed of fermentation. H2 and CO2 are the first gases to be produced; they can subsequently be utilized as substrates for CH4 production, reducing the gas volume. H2 can also be utilized to reduce sulfate to H2S, resulting from proteolytic fermentation2. As a result, N2, O2, CO2, H2 and CH4 constitute for 99% the volume of intestinal gas. The remaining 1% consists of NH3, H2S, volatile amino acids and short chain fatty acids.3 Each measurement was done in single repetition.
Acid/base consumption: the production of microbial metabolites in the colon reactors alters the pH. Without continuous pH control (through the addition of acid or base), the pH would exceed the fixed intervals. Consumption of acid/base is continuously monitored throughout the experiments

### (b) Microbial Community Activity:

Short-chain fatty acids (SCFA): The pattern of SCFA production is an assessment of the microbial carbohydrate metabolism (acetate, propionate and butyrate) or protein metabolism (branched CFA) and can be compared to typical fermentation patterns for normal GI microbiota. Quantitative analysis of the SCFA is done by means of capillary gas chromatography, coupled with a flame ionization detector (FID). The isolation of SCFA is performed by liquid-liquid extraction (De Boever et al 2000)
Lactate: the human intestine harbors both lactate-producing and lactate-consuming bacteria. Lactate is produced by lactic acid bacteria and decreases the pH of the environment, thereby also acting as an antimicrobial agent. It can also be rapidly converted into propionate and butyrate by other microorganisms. Determination of lactate concentrations was performed using the Enzytec™ kit (R-Biopharm).
Ammonium: Ammonium is a product of proteolytic degradation. Proteolytic fermentation results in the production of potentially toxic or carcinogenic compounds such as p-cresol and p-phenol. Determination of ammonium concentrations in the samples was done by colorimetric analysis, using the indophenol blue spectrophotometric (IPB) method.
Branched SCFA (BCFA; isobutyric acid, isovaleric acid and isocaproic acid) are markers of proteolytic fermentation. Quantitative analysis of the BCFA is done by means of capillary gas chromatography, coupled with a flame ionization detector (FID). The isolation of BCFA is performed by liquid-liquid extraction (De Boever et al 2000)
Uremic toxins: concentration levels uremic toxins (e.g. p-cresol, p-cresylsulfate, indole, indole-3-acetic acid, betaine, trimethylamine, trimethylamine-N-oxide, indoxyl, indoxyl sulfate, semialdehyde glutaric acid, uric acid, and urea) were determined using ultra-high performance liquid-chromatography coupled to high-resolution mass spectrometry and fluorescence detection.
Untargeted metabolomics: panel of microbial metabolites, including but not exhaustive to bile acids, amino acids were measured using Prodigest's Metakey platform

### Microbial Community Composition:

Community composition was measured using quantitative deep shotgun sequencing

### Description of Statistics

A two-tailed paired t-test was used for the statistical comparison of the different arms within each donor, each conditions and treatments. Differences were considered statistically significant if the p-value was less than 0.05.

### Method 3: Animal Experiments

### Animal Models

### Mouse Model

Animal experiments were performed at CarMeN Laboratory, Direction Départementale des Services Vétérinaires du Rhône. All experiments were carried out according to the guidelines laid down by the French Ministère de l'Agriculture and the European Union Council Directive for the Care and Use of Laboratory Animals. C57BL/6J mice were purchased from Janvier SA (Le Genest-Saint-Isle, France) and housed in an air-conditioned room with a controlled environment of 21 °C±0.5 °C and 60%-70% humidity, under a 12-hour light/dark cycle (light on from 07:00 to 19:00) with free access to food and water. Moderate CKD was induced by 5/6 nephrectomy with a two-step surgical procedure. Additional animals underwent sham surgery and served as control mice. Refer to FIG. 14 for an overview of the in-vivo animal experimentation.

### Animal Diet and Treatment

From week 0 to week 3, all animals were fed a standard rodent diet (SAFE A04 standard diet for rodents). Thereafter, animals were fed a standard diet or a customized version of a standard diet containing 1% (wt/wt) of the inventive nutritional symbiotic blend P1 and P2 for a total of 7 weeks. P1 intervention consisted of an ingredient blend containing Lactobacillus johnsonii NCC533, cellobiose, short-medium chain triglycerides containing butyrate and caprylate. P2 intervention consisted of an ingredient blend containing Lactobacillus johnsonii NCC533, cellobiose, pea GOS, short-medium chain triglycerides containing butyrate and caprylate. Custom diet with nutritional or symbiotic blend was made by dry mixing the powder version of SAFE A04 with the blend. Successful incorporation was verified including measurement of probiotic viability. After verification, custom diet was made into pellet as per usual SAFE procedure. During the pellet production of the custom diet, the viability of the probiotics decreased by 3 logs. Therefore, to ensure that animals received enough viable probiotics, additional probiotics were provided to the treated animals by dissolving powdered probiotic bacteria stock in sterile distilled water to reach a final concentration of approximately 1.8+8 CFU/ml. Viability of the probiotic in sterile drinking water was confirmed by plating. The probiotic water solution was changed every 2 days.

### Intraperitoneal Glucose Tolerance Test (IPGTT)

After an overnight fast, an intraperitoneal glucose tolerance test (glucose, 1 g/kg body weight) was performed. Blood glucose values were determined from a drop of blood sampled from the terminal portion of the tail, using an automatic glucose monitor (Accu-Check Performa, Roche, Meylan, France).

### Urine Parameters

Urine was collected over 24 hours in metabolic cages to evaluate urine output, and markers of kidney function, such as protein, creatinine, and albumin levels, and for metabolomics analysis. Markers of kidney function were measured using commercial assays. Urine metabolomics was measured using reverse-HPLC coupled to a fluorescence detector or using Prodigest's MetaKey® platform.

### Biochemical & Metabolomics Measurements

Uremic toxins were quantified in plasma and urine by using reverse-HPLC coupled to a fluorescence detector or using Prodigest's MetaKey® technology. The plasma or serum concentration of creatinine, cholesterol, triglycerides, free fatty acids, adiponectin, insulin, Cystatin C, and urea was determined using commercial assays. Serum metabolomics was performed using Prodigest's MetaKey® platform.

### Kidney Histology

In both sham and nephrectomised mice, the remnant kidney was removed at sacrifice. Kidneys were harvested and decapsulated. Kidney histological lesions were analyzed after Haemotoxylin & Eosin Staining (HES) and Sirius red staining. Briefly, the kidneys were fixed for 24 hours in 4% formalin and embedded in paraffin after conventional processing. The histology slides were independently examined on a blinded basis, for the level of interstitial inflammation, interstitial fibrosis and glomerulosclerosis.

### Ileum Histology

Ileum samples were collected during the end of the experiments. Ileum were fixed for 24 hours in 4% formalin and embedded in paraffin after conventional processing. The sections were then immunofluorescence stained with rabbit antibodies against occludin followed by goat anti-rabbit secondary antibodies. Images were captured using confocal microscope and the relative fluorescence intensities of occludin were quantified using image J software.

### Statistical Analyses

In each experiment, multiple mice were analyzed as biological replicates. Dot plots with a linear scale show the arithmetic mean. Bar graphs are expressed as the mean±standard error of the mean (SEM). GraphPad Prism version 9 was used for statistical analyses. For comparisons between two groups, significance was determined using the two-tailed Student's t test or nonparametric Mann-Whitney test. For comparisons among more than two groups, one way (ANOVA) followed by uncorrected Fisher's LSD tests. Differences were noted as significant at p≤0.05.

### Rat Model

The rat model experiments were performed at Grubra, a fully AAALAC accredited unit, and all animal experiments were conducted in accordance with Gubra's bioethical guidelines, which are fully compliant to internationally accepted principles for the care and use of laboratory animals. All experiments are licensed by the Danish Animal Experimentation Council. The 5/6 nephrectomy (Nx) was conducted in a two-step surgical procedure under isoflurane anesthesia in Wistar RjHan:WI rats (Janvier, France).

### Animal Diet and Treatment

Animals were treated in accordance with the following. At week −4, the two step nephrectomy (Nx) procedure was started. Starting at day −2, the animals were fed a standard rodent diet (SAFE A04 standard diet for rodents). At day 1, the animals were fed either the standard rodent diet or a customized diet in accordance with their group. The customized diet included the cellobiose and short-medium chain triglycerides containing butyrate and caprylate and the Lactobacillus johnsonii NCC533 (at 10⁸ CFU) was administered by gavage. This regime was administered once per day for 8 weeks.

### Example 1—Identification of Optimal Symbiotics to Improve the Build-Up of Uremic Toxins

### In Silico Screening and Identification of Candidate Probiotic Based on the Absence of Uremic Toxin-Related Enzymes Encoded in the Genome of the Bacterial Strains.

The European Uremic Toxin Work Group has listed 90 compounds considered to be uremic toxins (Yavuz et al., 2005). In the present example, gut-derived and plasma-bound uremic toxins, including urea, trimethylamine N-oxide (TMAO), indole-3-acetic acid (IAA), indoxyl and p-cresol were examined. Bacterial metabolic pathways to produce these uremic toxins were compiled from public database like KEGG pathway (Kyoto Encyclopedia of Genes and Genomes) and literature.

The different uremic toxins biosynthetic routes were summarized, and each enzyme potentially catalyzing the different steps was depicted using their EC numbers (Enzyme Commission Number) (FIG. 1). Conversions of urea to ammonia (NH3) and carnitine or choline to TMAO only require one or two steps. Whereas conversions of tryptophan to IAA and indoxyl, and tyrosine to p-cresol require several steps. The first and the last reactions of each pathway were analyzed in detail to determine the capacity of each bacteria to synthetize the selected uremic toxins. In this example, enzymes involved in these pathways are termed as key enzymes in the following text.

In this example, suitable probiotic strains analyzed were as follows:
Bifidobacterium animalis subspecies lactis NCC 2818 (CNCM I-3446);
Bifidobacterium longum subspecies infantis NCC 341 (ATCC 15697 (T));
Bifidobacterium longum subspecies longum NCC 2705 (CNCM I-2618) (NCBI refseq; GCA_000007525.1);
Enterococcus faecium NCC 2768 (NCIMB 10415);
Lactobacillus johnsonii NCC 533 (CNCM I-1225) (NCBI refseq; GCA_000008065.1);
Lactococcus lactis NCC 2287 (CNCM I-4154);
Lacticaseibacillus paracasei NCC 2461 (CNCM I-2116);
Lacticaseibacillus rhamnosus NCC 4007 (CGMCC 1.3724);
Staphylococcus carnosus NCC 1052 (CNCM I-5400);
Staphylococcus carnosus NCC 971 (CNCM I-5398);
Streptococcus thermophilus NCC 2496 (CNCM I-3915);

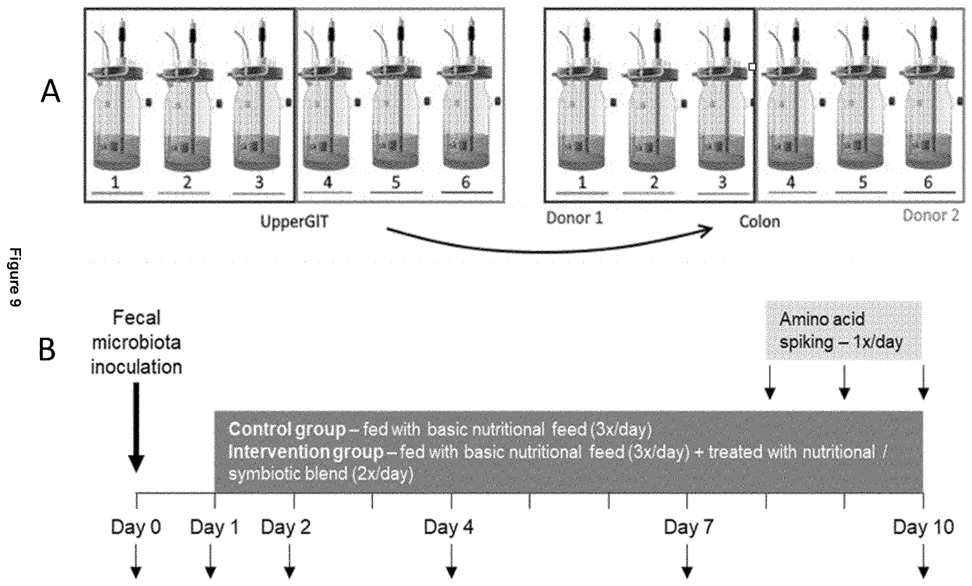

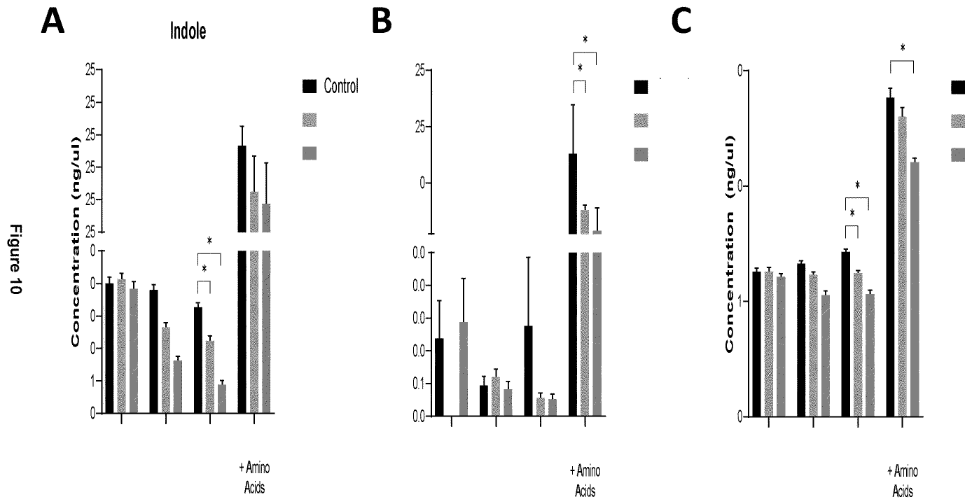

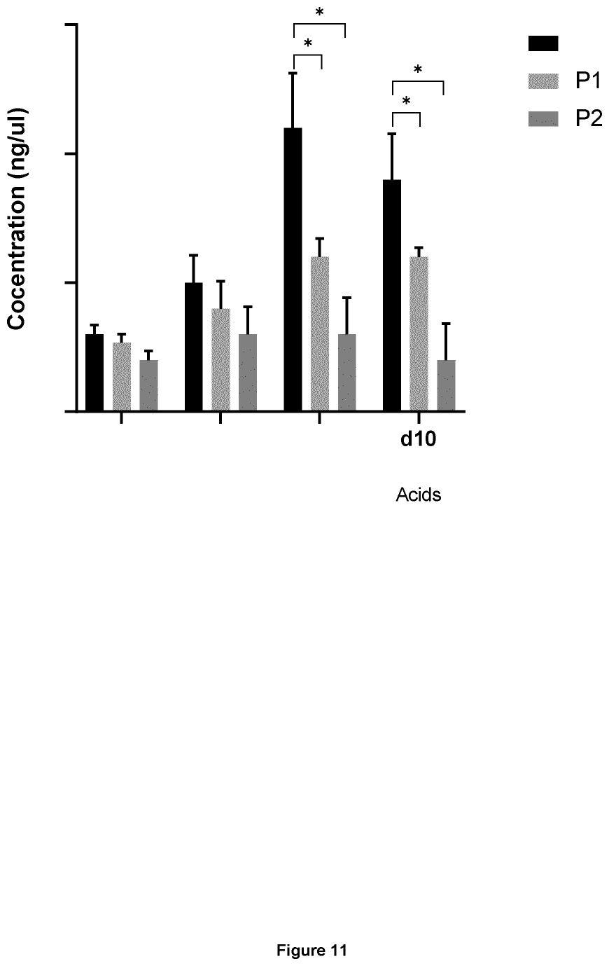

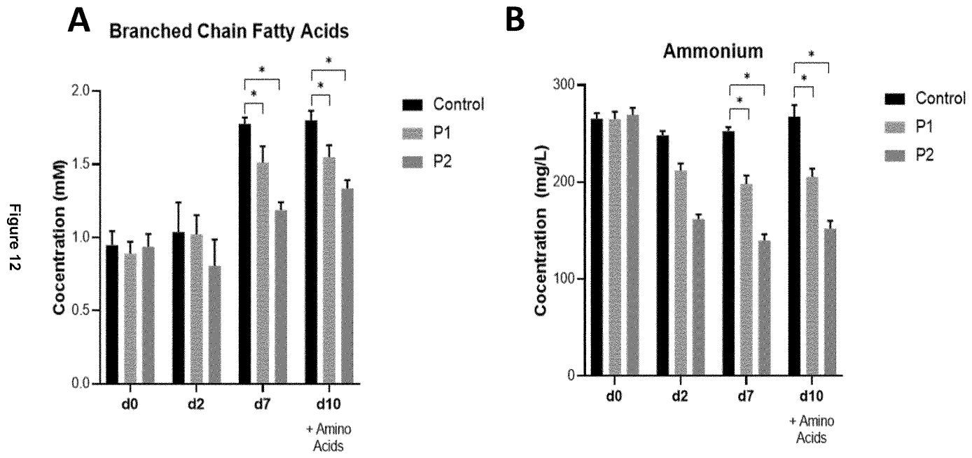

The presence of key enzymes catalyzing the synthesis of uremic toxins in the selected strains was depicted in FIG. 3. When building the reference protein sequences file, both sequence from SwissProt and proteins of bacteria that are phylogenetically as close as possible to the above probiotic strains were reviewed. As demonstrated in FIG. 3, L. johnsonii NCC 533 exceptionally does not have any of the key enzymes encoded in its genome and therefore predicted to have the lowest probability to produce any of the uremic toxins of concern. This strain was also chosen for the following in silico prebiotic carbohydrate selection and in vitro growth test.

### In Silico Screening and Identification of Prebiotic Carbohydrates Based on the CAZy Presence Encoded in the Genome of the Candidate Probiotics.

The candidate prebiotic carbohydrates were in silico selected based on their structure and likelihood to be degraded, metabolized, fermented or broken down by the candidate probiotic. As an example, the candidate prebiotic carbohydrates able to be degraded by the probiotic Lactobacillus johnsonii, NCC 533 (CNCM I-1225) (NCBI refseq; GCA_000008065.1), were analyzed and are shown in the following. Since enzyme activities are highly analogous, the findings can be transferred to the further suitable probiotic strains as analyzed above.

The genome sequence of the probiotic L. johnsonii was examined to identify all the Carbohydrate Active enZYmes (CAZy) it harbors. Table 1 summarized the CAZy relevant coding regions, CAZy family annotations, EC numbers of the encoded enzymes, functions, identified fiber ingredients and availability of carbohydrates substrates.

Lactobacillus johnsonii was shown to harbor all required Carbohydrate Active enZYmes (CAZY) enabling it to degrade the following fibers: α-galacto-oligosaccharides (raffinose family), galactomannans, 3-galacto-oligosaccharides, cello-oligosaccharides, cello-oligosaccharides, α-galacto-oligosaccharides (raffinose family) and ScFOS/Inulin.

### In Vitro Growth Test of the Candidate Probiotic and Carbohydrates.

In this example, growth tests were carried out and evaluated using the probiotic strain L. johnsonii to confirm the in silico selection and define optimal symbiotics to help improve uremic toxin retention.

Multiple carbohydrates were tested. Fiber poly- and oligomers containing galactose and glucose as their main constituents were selected. For example, galacto-oligosaccharides (GOS) in both α- and β-linked configurations, as well as mannans containing galactose as sidechains were tested. In addition, glucose polymers in the β-linked configuration included cello-oligosaccharides, purified β-glucans from barley in three molecular weights (high, medium and low), as well as the soluble fraction of enzymatically-hydrolyzed wheat fiber were tested. The constituting monomers (glucose, fructose, galactose) of each fiber ingredient were also analyzed in experiments in order to better understand which moiety can be metabolized by the probiotic strain.

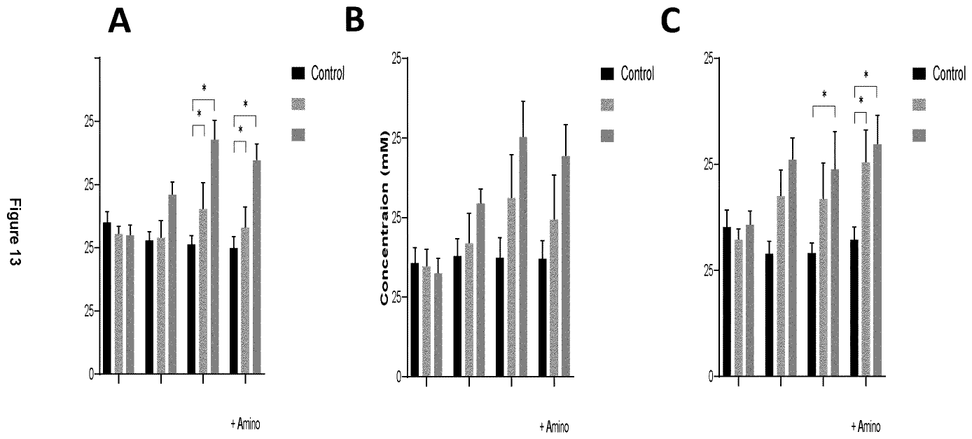

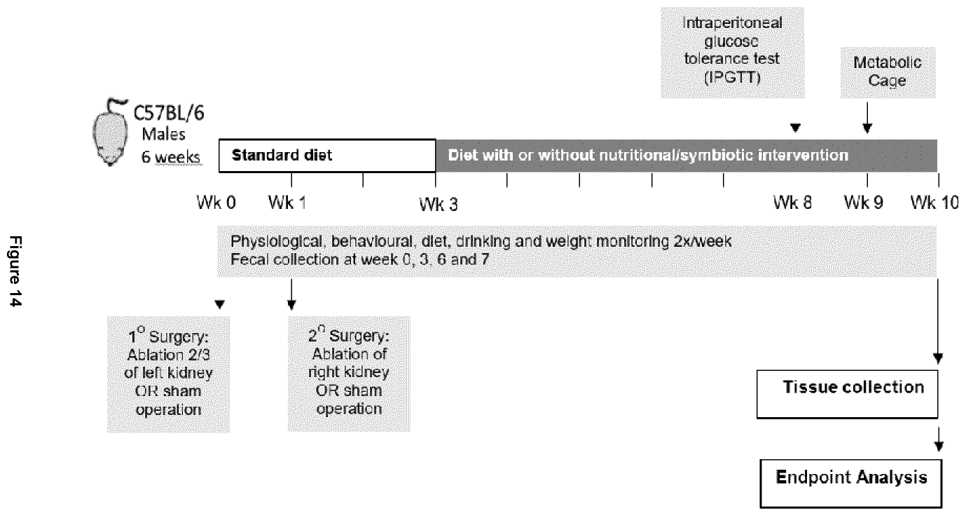

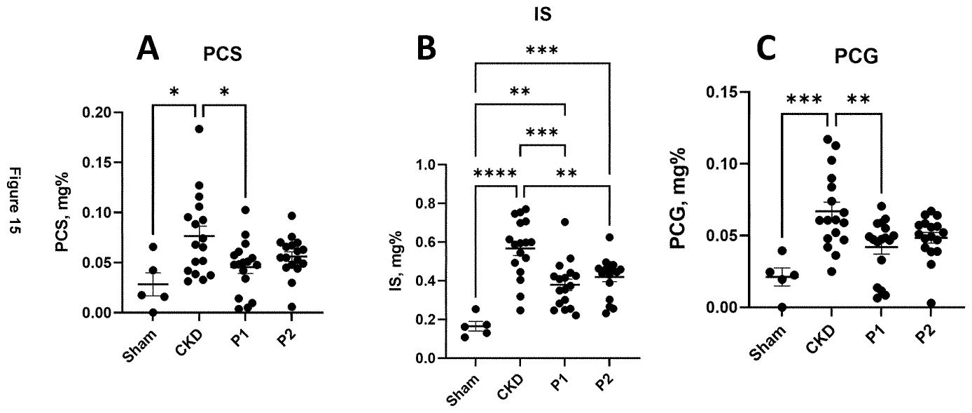

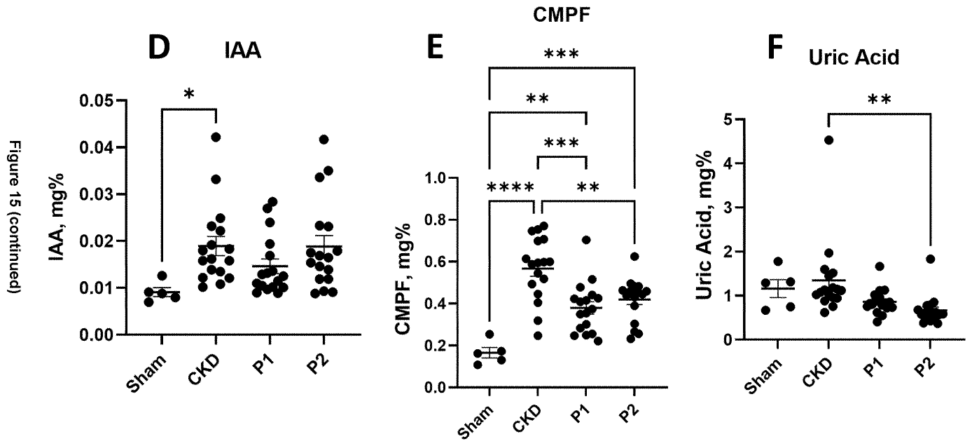

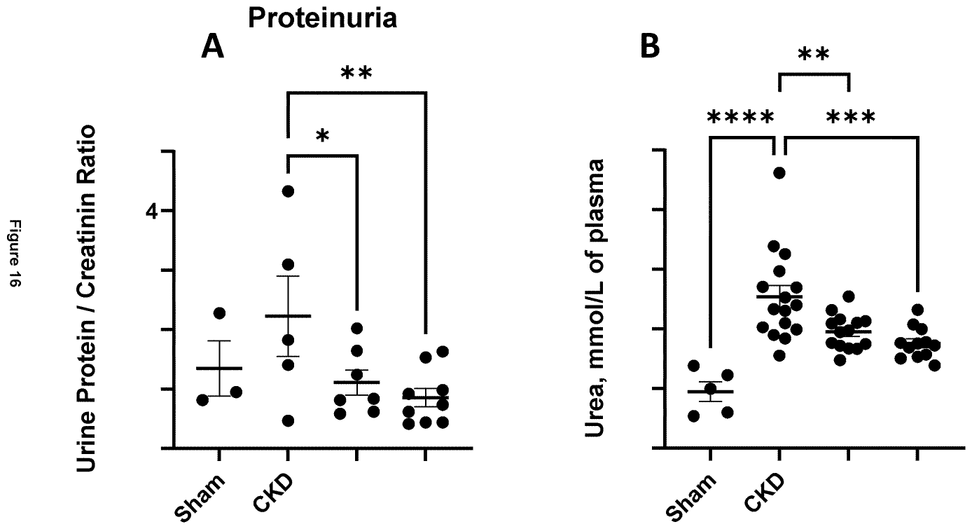

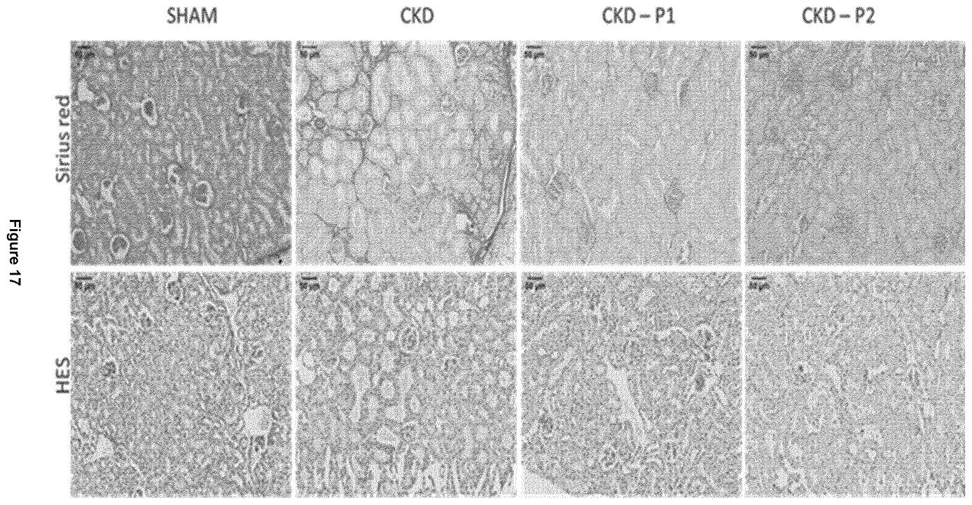

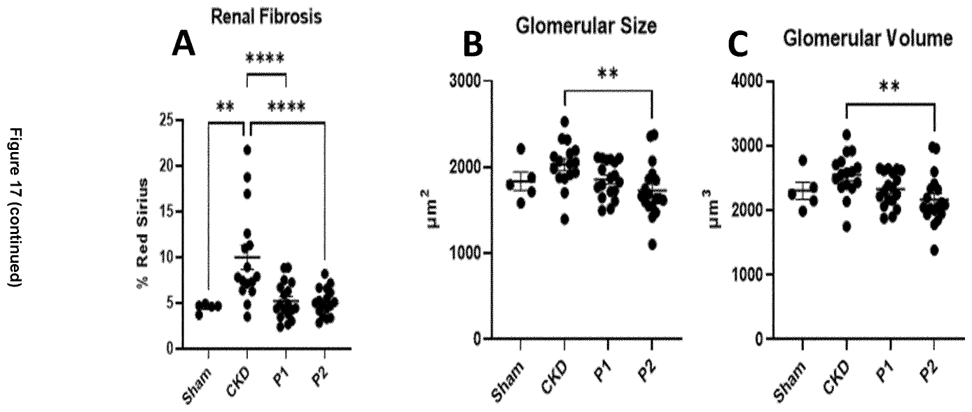

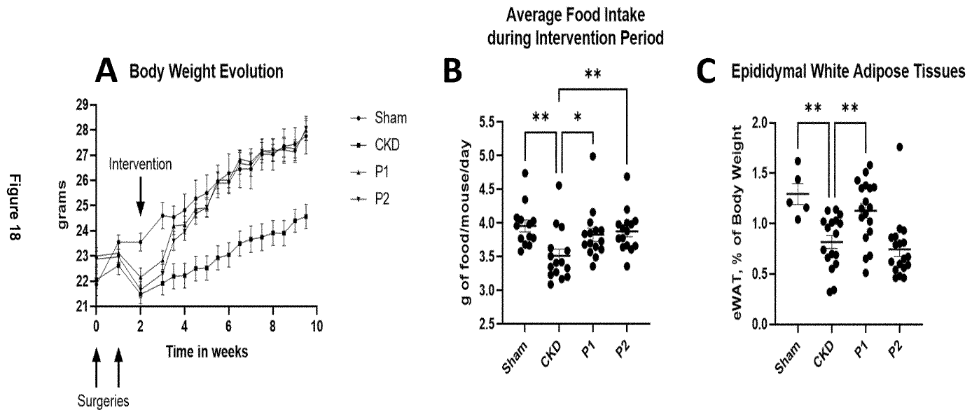

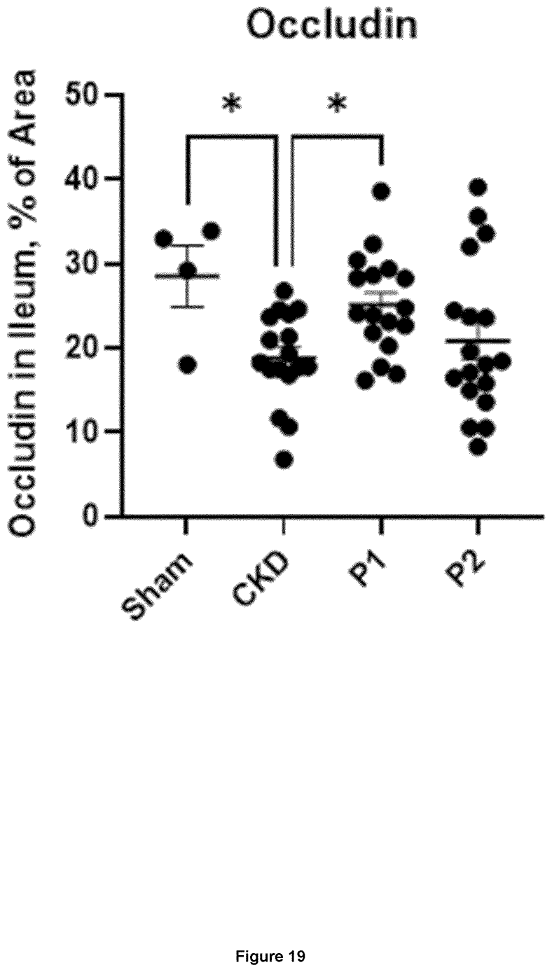

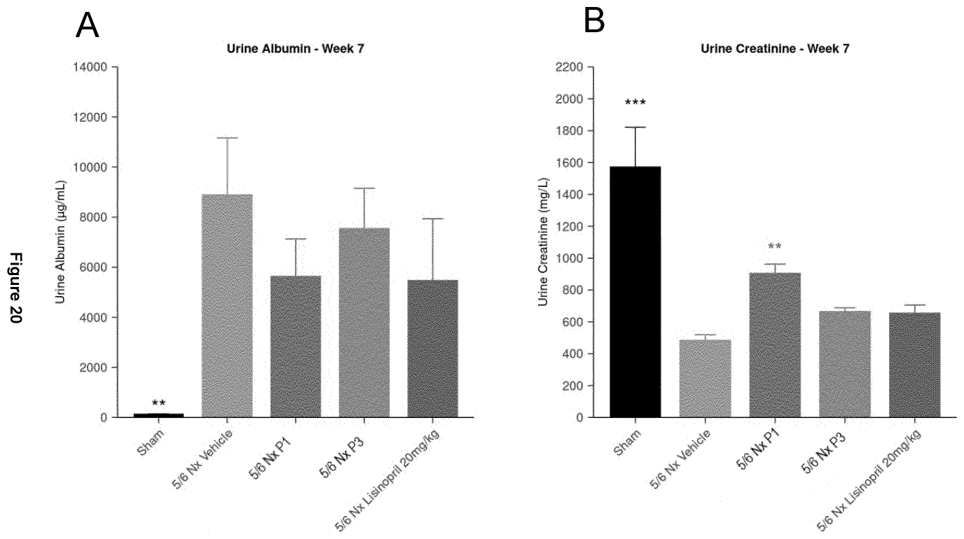

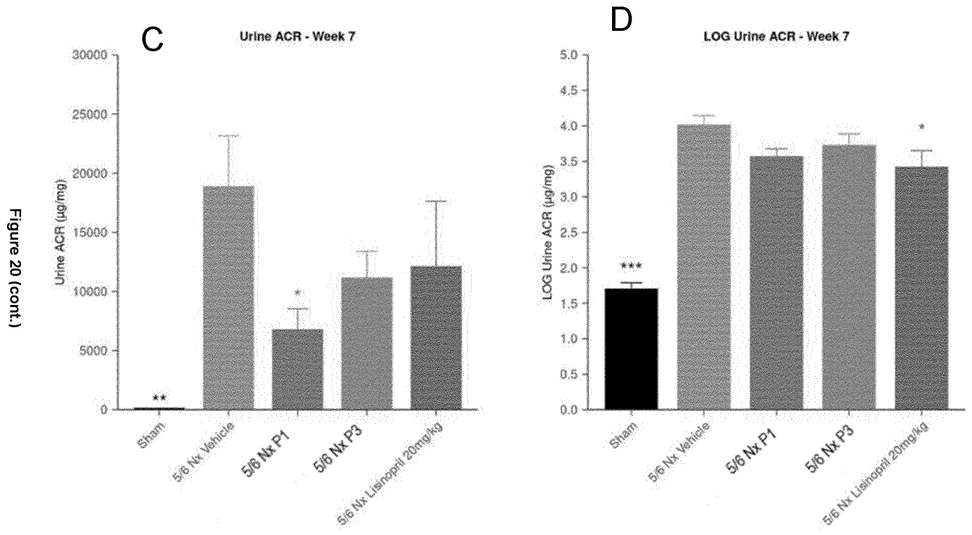

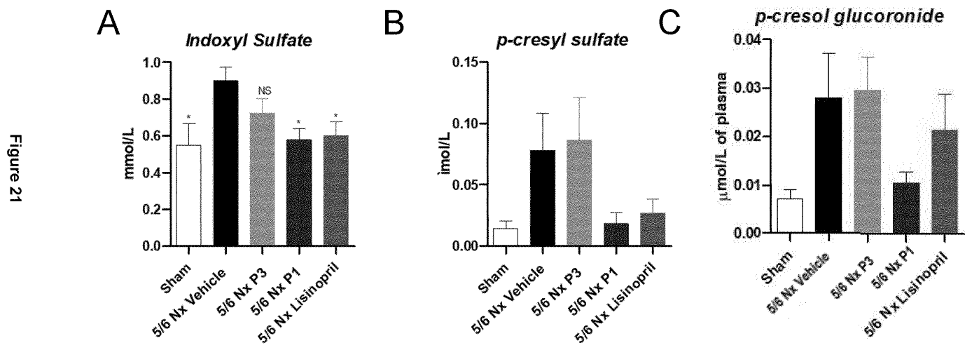

As indicated in FIG. 4, L. johnsonii NCC 533 was also able to grow on carbohydrates belonging to alpha-galacto-oligosaccharides, galactomannans or fructans families. Specifically, it is able to grown on commercially available fiber AlphaGOS® from pea or soy. For all those carbohydrates, a drop in pH was observed, indicating that L. johnsonii NCC 533 was sufficiently growing on these substrates. Similarly, L. johnsonii NCC 533 equally grew in the presence of commercial fiber Vivinal® GOS and BMOs (FIG. 5). On the other hand, Inulin, ScFOS and PHGG minimally support the growth of L. johnsonii NCC533 (FIG. 4). Furthermore, as shown in FIG. 6, L. johnsonii NCC 533 was able to grow efficiently on different carbohydrates belonging to cello-oligosaccharides family (from di- to tetra), especially on cellobiose and cellotriose. It was able to also grow effectively on cellotretraose and soluble hydrolyzed wheat and oat, containing cellobiose and cellotriose, but at lower extent or at later timepoint.

### Results

A total of 14 probiotic strains including the strains listed above and in FIG. 3 were investigated in this example. L. johnsonii NCC 533 was shown to not encode any of the key enzymes required to produce uremic toxins like ammonia, TMAO, IAA, indoxyl and p-cresol. Also, L. johnsonii NCC 533 encodes a large repertoire of enzymes to metabolize different carbohydrates. Based on its CAZy profile, 15 fibers were proposed for growth studies. L. johnsonii NCC 533 was shown to grow on several commercially available fibers, such as AlphaGOS® from soy or pea, Vivinal® GOS and BMOS. In addition, results confirmed that it indeed metabolized all the monomers forming those fibers (e.g. glucose, fructose, galactose). Therefore, it could be concluded that the strain is able to fully hydrolyze those fibers and use the constituting monomers as substrates for growth. Beta-galacto-oligosaccharides (such as Vivinal® GOS) are widely used today in infant nutrition and it has been consistently shown that they have a significant bifidogenic effect. This type of fiber is one of the few ingredients recognized as a prebiotic by the International Scientific Association of Prebiotics and Probiotics (ISAPP). Alpha-galacto-oligosaccharides, on the other hand, are emerging ingredients. They are by-products of the plant protein industry and are naturally found in leguminous seeds. Studies for alpha-galacto-oligosaccharides are scarce but have also consistently shown that they have a positive impact on the abundance of sachorrylutic and short-chain fatty acids producing bacteria such as bifidobacteria and lactobacilli.

L. johnsonii NCC 533 was also able to grow on cellobiose and related oligosaccharides (-triose and -tetraose). While cellobiose is commercially available in feed grade today, it is not the case for cello-oligosaccharides. Cello-oligosaccharides are dimer and trimer constituents of any cellulose-based fiber and/or mixed-linkage β-glucans in oat or barley. Therefore, an ingredient rich in cello-oligosaccharides can be manufactured by applying an optimized enzymatic hydrolysis to cellulose-based fibers/cereal or mixed-linkage β-glucans. Combined with other lactobacilli strains, cellobiose and/or cello-oligosaccharides in the form of β-glucan hydrolyzates have been shown to have the potential to exert synbiotic effects, especially in increased gut saccharolytic metabolism.

The other fibers tested (e.g. Inulin, ScFOS, PHGG, and high/medium/low-molecular weights of β-glucans) minimally support the growth of L. johnsonii NCC 533 which indicates that the strain is likely unable to fully hydrolyze the polymers down into the constituting monomers. However, based on the CAZy annotation for L. johnsonii NCC 533, it is possible that it can partially hydrolyze them into smaller fragments or oligomers. If the strain can indeed partially hydrolyze those substrates, it would imply that in a complex environment like the gut, a possibility of cross-feeding with other microbial inhabitants in the same ecosystem might occur. Overall these results suggest a beneficial role of co-administration of L. johnsonii NCC 533 and carbohydrates belonging from multiple families including alpha-galacto-oligosaccharides, such as pea GOS, and cello-oligosaccharides such as cellobiose.

### Example 2—In Vitro (ProDigest) Study

### Study Design

Several publications had demonstrated the dysbiotic profile of microbiota from CKD patients. Specifically, an altered microbial composition and function has been characterized in CKD patients compared to healthy individuals. Therefore, in this example, we tested capacity of the invention to correct the microbiome dysbiosis in CKD patients.

To do this, fecal microbiota were collected from healthy individuals (control) and patients with Chronic Kidney Disease (CKD). Table 3 provides an overview of the characteristics of the eight CKD patients and nine healthy adult donors that provided fecal microbiome for the in-vitro/ex-vivo experiments. The patients are part of the clinical study registered under ClinicalTrials.gov Identifier NCT04768309. Table 3 illustrates that characteristic of these donors. These fecal microbiota were then used on experimental set-up using a Prodigest's in-vitro/ex-vivo technology.

TABLE 3

| | Control | CKD | p |
|---|---|---|---|
| No. of patients | 9 | 8 | |
| Caracteristics | | | |
| Age (years) | 37 ± 8 | 55 ± 10 | <0.001 |
| Sex (F/M) | 2F/7H | 2F/8M | ns |
| BMI (kg/m²) | 23.6 ± 2.5 | 23.7 ± 2.8 | ns |
| Renal function | | | |
| creatinine (μmol/l | 85 ± 12 | 309 ± 50 | <0.001 |
| DFG ml/min/.73 m2 | 101 ± 10.4 | 18 ± 3 | <0.001 |
| proteinuria g/24 h | 0 ± 0 | 1.41 ± 1.03 | <0.001 |
| Inflammatory parameters | | | |
| CRP, mg/L | 2.08 ± 3.2 | 5.14 ± 8.57 | 0.39 |
| IL-6 | 1.12 ± 0.4 | 13.4 ± 31.28 | 0.28 |
| TNF alfa | 7.42 ± 2.8 | 18.58 ± 2.08 | <0.001 |
| Food intake | | | |
| proteins, g/kg/day | 0.95 ± 0.25 | 0.87 ± 0.20 | 0.45 |
| calories, kcal/kg/dau | 25.6 ± 6.4 | 22.46 ± 7.1 | 0.37 |
| Fibres | 17.13 ± 5.10 | 14.13 ± 6.33 | 0.31 |
| Uremic toxins | | | |
| IS, μg % | 9.8 ± 5.8 | 34.0 ± 16.1 | <0.001 |
| Free, IS, μg % | 0.2 ± 0.3 | 1.0 ± 0.5 | <0.001 |
| PCS, μg % | 15.3 ± 9.6 | 43.6 ± 20.2 | <0.001 |
| Free PCS, μg % | 0.4 ± 0.4 | 1.1 ± 0.7 | <0.001 |
| PCG, μg % | 0.1 ± 0.1 | 0.7 ± 0.8 | 0.06 |
| Free PCG, μg % | 0.1 ± 0.1 | 0.6 ± 0.8 | 0.07 |
| IAA, μg % | 3.4 ± 0.8 | 4.4 ± 1.4 | 0.09 |
| Free, IAA, μg % | 0.7 ± 0.1 | 0.9 ± 0.2 | <0.01 |
| CMPF, μg % | 11.9 ± 12.4 | 10.0 ± 13.1 | ns |
| HA, μg % | 21.0 ± 16.8 | 27.6 ± 13.6 | ns |
| Free HA, μg % | 7.3 ± 7.5 | 10.1 ± 6.0 | ns |
| Uric acid, μg % | 612.3 ± 177.6 | 806.5 ± 249.6 | 0.08 |

In this example, two studies were performed sequentially.

First, the difference in the fecal microbiome profile between healthy and Chronic Kidney Disease (CKD) adult donors, using Prodigest's short-term single-stage colonic simulation technology was assessed (see FIG. 7 and its description for further experimental details).

Second, a modified Prodigest's SHIME® technology was used to assess the impact of the new nutritional or symbiotic ingredients on the fecal microbiota from patients with Chronic Kidney Disease (CKD) (See FIG. 9 and its description for further experimental details).

For the second study, eight CKD donors were used and two ingredient blends (P1 arm, P2 arm, see below, compositions for P1 and P2 as indicated below) and a control were tested. Thus, for each CKD microbiota, three conditions were implemented:
Control (CTRL) arm: SHIME unit fed with basic nutritional medium, which was used to determine the baseline parameters of each donor for comparison with the treated arms.
P1 arm: SHIME unit fed with basic nutritional medium during one cycle per day (entrance at 1 h in colon). During the other two cycles, this SHIME unit was fed with a carbohydrate-depleted nutritional medium, and supplemented with nutritional or symbiotic ingredient composed of cellobiose, butyric acid and caprylic acid and probiotic L. johnsonii NCC 533.
P2 arm: SHIME unit fed with basic nutritional medium during one cycle per day. During the other two cycles, this SHIME unit was fed with a carbohydrate-depleted nutritional medium, and supplemented with cellobiose, pea GOS, butyric acid and caprylic acid and probiotic L. johnsonii NCC 533.

Additionally, the amino acid mix was added reaching a colonic concentration of 2.5 g/L to provide substrates to test if the invention is able to ameliorate over-production of uremic toxins and precursors. The amino acid mix consisted of 23% L-phenylalanine, 20% L-tryptophane, 23% L-tyrosine, 10% L-carnitine and 23% choline. The amino acid mix was added on d8 (entrance at 1 h in colon), d9 (entrance at 1 h in colon) and d10 (entrance at 1 h in colon) of the experimental run during one feeding cycle per day, and this for both the control and treatment arms of each donor.

### Results

Fecal Microbiota from CKD Patients Displayed Amino Acid and Protein Dysmetabolism

To validate whether the in-vitro/ex-vivo model is a reliable model to test the efficacy of the invention to modulate CKD microbiome, fecal microbiota from CKD patients and healthy donors were inoculated in Prodigest's system.

As seen in FIG. 8, CKD microbiota showed higher production of uremic toxin precursor, such as p-cresol, especially in the presence of excess amino acid substrate. In terms of proteolytic activity, CKD microbiota also showed higher concentration of proteolytic markers, such as branched chain fatty acids.

Nutritional or Symbiotic Blend Reduced the Production of Uremic Toxin Precursor by the Microbiota from CKD Patient Donors

The ability of the invention to ameliorate the over-production of uremic toxins and their precursors were evaluated in the presence of low amino acid (AA: day 0 to day 7) and additional AA substrates (day 8 to day 10). In this example, three uremic toxin precursors, including indole, p-cresol and trimethylamine (TMA) were shown (see FIG. 10). It is hypothesized that the addition of AA will induce further increase in production of uremic toxins and their precursors by the fecal microbiota.

Indole can be metabolized by gut microbiota using tryptophan as a precursor molecule; thereby converting tryptophan first into tryptamine and indole pyruvic acid, and then converting indole pyruvic acid into indole. It is known that indole has profound effects on the gut microbial composition, microbial metabolism, the host's immune system, the host-microbiome interface, and host immune system-intestinal microbiota interactions. Once produced by intestinal bacteria, indole is absorbed into the portal circulation and enters the liver. Hepatic hydroxylation of indole results in 3-hydroxy-indole (indoxyl), of which the majority is then sulfonated into indoxyl sulfate. Indoxyl sulfate is considered as uremic toxins and has been most frequently implicated as a contributor to kidney diseases progression and cardiovascular complications. Moreover, indoxyl sulfate has also been assigned adverse effects on bones and the central nervous system. The day prior to administration of the AA formulation (d7), a significant lower indole concentration was detected for both P1 and P2 compared to the non-treated control group (FIG. 10). Upon administration of the AA formulation, a strong increase in indole concentration was observed for all donors, which is in line with the fact that tryptophan as indole precursor molecule was provided.

P-cresol, as a phenolic compound, is a microbial metabolite that is produced from tyrosine, thereby forming the intermediates 4-hydroxy-phenylpropionic acid and 4-hydroxy-phenylacetic acid. In the liver, p-cresol is sulfated and transformed into the toxic metabolite p-cresyl sulfate. Mechanistic studies have linked this particular metabolite to oxidative stress, endothelial dysfunction, proximal tubular injury and insulin resistance. In this respect, a positive relationship between p-cresyl sulfate levels and overall mortality, cardiovascular disease, and progression of CKD has been demonstrated. As seen in FIG. 10, p-cresol, prior to AA spiking (period d0-d7), a lowering effect was defined for P1 and P2 treatment compared non-treated control. As expected, administration of the AA formulation resulted in increased concentration levels for p-cresol in all donors in the blank control group compared to the preceding period. Interestingly, both P1 and P2 was able to ameliorate the over-production of p-cresol compared to blank control.

Trimethylamine (TMA) is a metabolite that is being produced by the gut microbiota in the intestinal lumen, with various dietary quaternary amines as precursor molecules. These precursor molecules mainly include choline and carnitine, but also betaine, γ-butyrobetaine, and other choline-containing compounds. In vivo, produced TMA is rapidly absorbed into the portal circulation by passive diffusion and then oxidized to trimethylamine-N-oxide, by the action of hepatic flavin containing monooxygenases. Trimethylamine-N-oxide (TMAO) is involved in oxidative stress, inflammation, cardiac fibrosis, endothelial injury, and platelet inactivation. As such, TMAO has been assigned a biological role in several chronic non-communicable diseases, including CKD. As shown in FIG. 10, during the period of d0 to d7, an increasing concentration level of TMA was observed for the blank control group. P1 and P2 showed lower concentration levels compared to the blank control group, with the largest changes observed for P22. Upon administration of the AA formulation, a clear increase in trimethylamine concentration was observed for all donors and experimental conditions, which relates to the increased availability of substrate to produce TMA. When performing statistical analysis across all donors at timepoint d10, a significant impact of P2 compared to the blank control was calculated.

Overall, this data highlights the efficacy of the invention to correct the amino acid dysmetabolism in the microbiota of CKD patients. Notably, the invention is efficacious in conditions with low and normal levels of amino acid suggesting the benefits of the invention to be used by different patients with different dietary restrictions such as low protein diet.

### Nutritional or Symbiotic Blend Decreased Production of Urea

Urea is a metabolite that is produced through the urea cycle, which is the metabolic pathway that eliminates excess of endogenous and exogenous nitrogen from the body by detoxification of ammonia into urea. In this respect, typical nitrogen sources include amino acids such as ornithine, arginine, aspartic acid, glutamic acid, etc. Although the urea cycle mainly takes place in the liver, also gut microbiota effectuates a mitochondrial urea cycle. In CKD patients, higher urea concentrations are typically observed, which are known to significantly modify the microbiota in the gut, causing a decrease in bacterial strains that produce anti-inflammatory and fuel molecules and an increase in bacterial strains that can metabolize urea, but also produce uremic toxins including indoxyl sulfate and p-cresol sulfate. Moreover, high urea concentrations may also cause an increased gut permeability and a toxic environment that induces the colonization of bacteria that express ureases and uricases to reduce urea to ammonia. Ammonia increases gut pH, facilitating the increase of pathogen bacteria. As the amino acid formulation includes various sources of nitrogen, spiking this formulation may induce increased urea concentration levels during the colonic incubations. It should, however, be noted that the amino acids that were part of the concerned formulation have not been reported to be specifically involved in the urea cycle. In this respect, a general alteration of the microbial composition by the amino acid spiking, with particular alterations of those bacterial strains that metabolize urea, may define the final outcome for the observed urea concentration levels.

As noted in FIG. 11, during the period of d0 to d7 as well as d10, a trend of an increasing concentration profile was. Lowering effects on the concentration levels of urea were noted for both P1 and P2, with P1 having the strongest impact. As expected, administration of the AA formulation had in general limited impact on the urea concentration levels.

This data highlights the efficacy of the invention to reduce the production of uremic toxins by the gut microbiota. Furthermore, the invention may provide relief on gut alteration and symptoms associated with over-production of urea including intestinal permeability and inflammation.

### Nutritional or Symbiotic Blend Increased Reduced Markers of Proteolytic Fermentation

Both the production of ammonium and branched SCFA (sum of isobutyrate, isovalerate and isocaproate) result from protein degradation and reflect proteolytic activity of the gut microbiota. As the latter has been associated with direct and indirect adverse health effects (for instance colon carcinogenesis), a reduction in ammonium/branched SCFA production is considered as beneficial.

As noted in FIG. 12, branched SCFA levels increased throughout the experiment in all arms of each donor. Supplementation of both ingredient blends (P1 and P2, see above) systematically decreased branched SCFA production across all donors compared to control (see also above). This was also observed in the average effects across the eight donors on day 7 and day 10, where branched SCFA production was significantly reduced in the treatment arms as compared to the blank control, with the strongest effect observed for P2.

Similar effects were also observed in terms ammonium levels (FIG. 12), with both P1 and P2 strongly reduced ammonium production throughout the entire experiment. This was also observed on day 7 and day 10, with both treatments significantly reducing ammonium levels as compared to the blank control when averaged over the eight selected donors. Strongest effects were observed following repeated administration of P2, resulting in an even stronger significant reducing effect as compared to P1 on day 7 and 10.

This data supports the efficacy of the invention to correct the dysbiosis in CKD microbiota, particularly protein dysmetabolism.

### Nutritional or Symbiotic Blend Increased Production of Short Chain Fatty Acid (SCFA)

SCFA production results from carbohydrate metabolism in the colon and is related with various health effects. The most abundant SCFAs are acetate, propionate, and butyrate. SCFAs are well-known to play a crucial role in gut health. Acetate can be used as an energy source for the host and as a potential substrate for lipid synthesis in the body. Moreover, it is an important by-product in the synthesis of butyrate and can exert antimicrobial effects against pathogens. However, the health-promoting effects are mainly attributed to propionate and butyrate, which act as the main energy sources for the gut epithelium and have shown protective effects against inflammation and colon cancer. The former is known to also be transported to the liver, where it has a cholesterol-lowering effect in plasma and positively affects glycemic control. In summary, beneficial effects of the investigated substrates on SCFA production therefore include an increase of acetate, propionate and/or butyrate production. For optimal interpretation, SCFA levels for both test conditions are presented for each of the different SCFA.

Acetate is one of the key metabolites in the human gut and is thus produced by a wide range of gut microbes including among many others Bacteroides spp. (phylum Bacteroidetes) and bifidobacteria. As seen in FIG. 13, administration of both ingredient blend (P1 and P2) increased the overall acetate production compared to the blank control on average across all donors compared to control. P2 exerted the strongest effect. P1, P2 and control were as defined above.

Propionate can be produced by a wide range of gut microbes, with the most abundant propionate producers being Bacteroides spp. (phylum Bacteroidetes), Veillonella (phylum Firmicutes) and Akkermansia muciniphila (phylum Verrucomicrobia). It followed that treatment effects on propionate production were both donor- and product-dependent. Treatment with P1 and P2, it was observed an increasing levels of propionate levels but the levels did not reach significance, owing to some variable response among the 8 donors.

Butyrate is produced by members of the Clostridium clusters IV and XIVa (phylum Firmicutes). In a process referred to as cross-feeding, these microbes convert acetate and/or lactate (along with other substrates) to the health-related butyrate. It was observed that both P1 and P2 had a strong stimulatory effect on butyrate production in specific donors. When averaged over the eight selected donors, only treatment with P2 significantly enhanced butyrate production towards the end of the control/treatment period prior to AA spiking (day 7), though a similar trend was observed for P1. Finally, following AA spiking during the final 3 days of the experiment, also P1 significantly enhanced butyrate levels compared to the blank control when averaged over the different donors, reaching similar levels as compared to P2 administration.

Overall, the above example supports the efficacy of the invention to improve saccharolytic activity of the CKD microbiota and enhanced the production of beneficial metabolites such as SCFA.

### Example 3—In Vivo Animal Study

### Study Design

To test the efficacy of the invention to ameliorate uremic toxin build-up and related symptoms and clinical outcomes, the animal model 5/6 nephrectomy was used in the study. Kidneys were ablated during 2-step surgery procedure to reduce kidney function (reminiscent of human CKD stage 3 and above). Additional animals were sham-operated and served as non-CKD animal control (refer to FIG. 14 and its description for overview of the experimental design and analysis).

From week 3 to week 10, the CKD animals were fed a diet with or without the nutritional or symbiotic blend interventions. In this example, two nutritional/symbiotic blend combinations (P1 and P2) were tested. P1 intervention consisted of an ingredient blend containing Lactobacillus johnsonii NCC533 10⁸ (this is corrected from the concentration reported in U.S. Provisional Application Nos. 63/439,638 and 63/480,729, which reported the incorrect concentration), cellobiose 1%, short-medium chain triglycerides containing butyrate and caprylate 1%. P2 intervention consisted of an ingredient blend containing Lactobacillus johnsonii NCC533 10⁸ (this is corrected from the concentration reported in U.S. Provisional Application Nos. 63/439,638 and 63/480,729, which reported the incorrect concentration), cellobiose 1%, pea GOS 1%, short-medium chain triglycerides containing butyrate and caprylate 1%.

At week 8, glucose tolerance test was performed. At week 9, mice were placed in metabolic cages to collect urine for uremic toxin and kidney parameter analysis. At week 10, the mice were sacrificed and different tissues were collected for subsequent analysis.

### Results

Treatment of Uremic Mice with a Nutritional or Symbiotic Blend Improved Plasma Concentration of Clinically Relevant Uremic Toxins

To demonstrate the importance of the inventive nutritional or symbiotic blends in improving uremic toxins, a 5/6 nephrectomy or the remnant kidney model was used. The model represents one of the most used animal models of the progressive renal failure by reduced nephron number, mimicking the condition seen in patients with CKD. As seen in FIG. 15, animals that underwent nephrectomy (Group CKD) showed significantly higher plasma levels of clinically relevant uremic toxins, such as p-cresyl sulfate (PCS), indoxyl sulfate (IS), P-cresylglucuronide (PCG), Indole acetic acid (IAA), 3-Carboxy-4-methyl-5-propyl-2-furanpropionate (CMPF) and uric acid compared to animals with intact kidney function (Group Sham). This demonstrates that the CKD animals are in uremic state and thus a good model to investigate the impact of uremic treatments.

As similarly seen in FIG. 15, elevated plasma uremic toxins were significantly reduced in CKD animals treated with nutritional or symbiotic blend (Group P1 and P2, see above) compared to CKD animals; thus, highly supporting the efficacy of the blend on its intended use.

### Nutritional or Symbiotic Blend Stabilize Kidney Function in Nephrectomised Animals

We further investigated the benefits of the nutritional or symbiotic blend on kidney function and structure. To determine the progression of functional damage in the remnant kidney, proteinuria and blood urea levels were evaluated. Furthermore, progressive microscopic damage was also evaluated by histology.

Proteinuria is a symptom typically seen with conditions affecting the kidneys. Too much protein in the urine means that the kidneys' filtering mechanism the glomeruli are not working properly and are allowing too much protein to escape in the urine. Proteinuria was measured by calculating the ratio of protein over creatinine in the urine.

Blood Urea Nitrogen level in the blood is an indicator of kidney function. Urea is produced as by-product in the liver when protein is metabolized. Healthy kidneys filter urea out of the body through urine. High urea levels generally indicate acute or chronic kidney disease or kidney failure. Urea is also considered as one of the clinically relevant uremic toxins.

As seen in FIG. 16, nephrectomised animal (Group CKD) showed significantly higher proteinuria compared to non-CKD animals (Sham), confirming the progression of kidney damage. Interestingly, CKD animals treated with nutritional or symbiotic blends (Group P1 and P2, see above) showed significantly improved proteinuria and blood urea levels compared to non-treated CKD animals.

As further shown in FIG. 17, progressive kidney histology damage was evident in CKD animals compared to sham animals. Animals treated with nutritional or symbiotic blend (Group P1 and P2, see above) showed significantly better histology, as demonstrated by lower fibrosis and improved size and volume of glomeruli compared to non-treated CKD animals.

Overall, the data supports the potential benefit of the blends to not only reduce levels of uremic toxins in the blood but also in helping stabilize kidney function as shown as less appearance of markers of kidney damage progressions.

Nutritional or Symbiotic Blend Reduces Loss of Appetite, Weight Loss and Fat Mass Associated with Uremic Toxins and Chronic Kidney Disease

Uremic toxicity negatively affects multiple organ systems and metabolic pathways leading to organ damage and manifestation of symptoms including neurological conditions and protein energy wasting. As such, we tested the ability of the nutritional or symbiotic blend to improve some of the detrimental consequences of the build-up of uremic toxins; this includes food intake and wasting of energy reserves, such as muscle and adipose fat.

As shown in FIG. 18, CKD animals treated with the P1 and P2 intervention (see above) showed significantly improved body weight evolution and normalization of food intake to the same level as non-CKD sham animals. The energy intake was not significantly different between the two groups. Furthermore, treated animals, particularly those treated with P1 showed reduced loss of fat mass observed in CKD, as shown by better adipose reserves as seen by improved epididymal white adipose tissue (eWAT) compared to non-treated CKD animals.

Overall, the data supported the potential benefits of intervention to alleviate uremia-related symptoms including loss of appetite, weight loss and protein energy wasting.

### Nutritional or Symbiotic Blend Improved Intestinal Barrier Dysfunction.

One of the detrimental effects of dysbiosis and build-up of uremic toxins in the gut is induced alteration in intestinal barrier function. Altered intestinal permeability may also induce vicious cycle of uremic toxin build-up in the systemic circulation.

As such, we further tested the possibility of the new ingredient blend to improve some of the intestinal barrier dysfunction described in CKD. As seen in FIG. 19, CKD animals showed impaired protein expression of an important tight junction, occludin, compared to non-CKD animals. Intact tight junctions are important to prevent excessive translocation of gut contents and molecules from going to systemic circulation.

Hence, the data supported the potential benefits of intervention to not only prevent the build-up of uremic toxins but to also alleviate some of the intestinal barrier dysfunction associated with the disease.

### Example 4—In Vivo Animal Study

### Study Design

To further confirm the kidney protective effect shown by the mouse model, 5/6 nephrectomized rats were treated with an ingredient blend containing Lactobacillus johnsonii NCC533, cellobiose, short-medium chain triglycerides containing butyrate and caprylate.

9 weeks old Wistar RjHan:WI rats (n=84) underwent 2/3 nephrectomy of the right kidney at week −4 and full nephrectomy of left kidney at week −2. 12 Additional rats underwent sham-surgery. Rats were randomized based on plasma urea, plasma creatinine, and body weight measured the week prior to study start into the following treatment groups:
(1) Sham (n=12);
(2) 5/6 Nx Vehicle (n=18);
(3) 5/6 Nx P1-rat Diet (P1-rat intervention consisted of an ingredient blend containing cellobiose 1%, short-medium chain triglycerides containing butyrate and caprylate 1% that was administered in food and Lactobacillus johnsonii NCC533 10⁸ was administered by gavage) (n=18);
(4) 5/6 Nx P3 Diet (P3 intervention consisted of an ingredient blend containing cellobiose 0.3%, short-medium chain triglycerides containing butyrate and caprylate 0.3% that was administered in food and Lactobacillus johnsonii NCC533 10⁸ was administered by gavage) (n=18); and
(5) 5/6 Nx Lisinopril 20 mg/kg.

Lisinopril is an angiotensin-converting enzyme inhibitor (ACEi) that is a standard care for nephroprotection.

Diets were administered for 8 weeks. Water intake was measured on days 9, 10, 11, 25, 26, 27, 39, 40, and 41. Urine was sampled for analysis of albumin and creatinine at week 7. At termination, plasma was sampled for analysis of urea, creatinine, indoxyl sulfate and p-cresyl sulfate.

### Results

A symbiotic composition comprising Lactobacillus johnsonii NCC533 10⁸, cellobiose 1%, short-medium chain triglycerides containing butyrate and caprylate 1 was able to reduce plasmatic uremic toxin concentrations, specifically indoxyl sulfate and p-cresyl sulfate (see FIG. 21).

As seen in FIG. 20 (C), nephrectomised animal (Group 5/6 Nx Vehicle) showed higher proteinuria compared to non-CKD animals (Sham), confirming the progression of kidney damage. 5/6 Nx animals treated with nutritional or symbiotic blends, specifically the P1 blend, showed significantly improved proteinuria levels compared to non-treated CKD animals.

### Example 5—Clinical Trial Synopsis (Summary)

In the following, the clinical trial synopsis s disclosed forming basis for the human clinical trial.

General Information

| Intervention Area/ | Chronic kidney disease/kidney-related complications and | | | |
|---|---|---|---|---|
| Group Area | manifestations | | | |
| Trial title | A prospective, randomized, double-blind, placebo-controlled, multi- | | | |
| | centre, multi-country, trial to study the effects of a nutritional food | | | |
| | for special medical purpose (FSMP) product (CmKD) containing | | | |
| | synbiotics (e.g., a prebiotic [for example cellobiose] and a probiotic | | | |
| | [for example L johnsonii NCC533]) and gut/kidney energetic | | | |
| | substrates (e.g., short and medium chain triglycerides containing | | | |
| | butyric (C4) and caprylic (C8) acid)) on reducing uremic toxins in | | | |
| | people with impaired kidney function. | | | |
| Investigational | Active Formula: CmKD is a specific synbiotic blend combined | | | |
| Products being tested | with a gut/kidney energetic substrate (TBC: tentative Cellobiose + | | | |
| | L johnsonii NCC 533 + C4/C8); analogous to P2, described | | | |
| | herein before (e.g., Lactobacillus johnsonii NCC533, cellobiose, | | | |
| | pea GOS, short-medium chain triglycerides containing butyrate | | | |
| | and caprylate; or a composition analogous to P1 (e.g. | | | |
| | Lactobacillus johnsonii NCC533, cellobiose, short-medium chain | | | |
| | triglycerides containing butyrate and caprylate). | | | |
| | Placebo: A placebo matching to the test product is used as control | | | |
| | (canola oil and maltodextrin having the caloric equivalent). | | | |
| Trial Rationale / | Can prevention of deleterious uremic toxin production and/or | | | |
| Trial hypothesis | accumulation, be achieved by providing a FSMP containing | | | |
| | synbiotics (combination of prebiotic [non-digestible food ingredients | | | |
| | that stimulate the growth of healthy bacteria in the gut] and probiotic | | | |
| | [live microorganisms] which is believed to modify the gut microbiota | | | |
| | and help restore a healthy gut flora) and a gut/kidney energetic | | | |
| | substrate (short and medium chain triglycerides containing butyric | | | |
| | (C4) and caprylic (C8) acid))? | | | |
| Objectives (primary, | Primary efficacy outcomes assesses change from baseline in | | | |
| secondary, tertiary, | serum levels of total and free p-cresyl sulfate (pCS), p-cresyl | | | |
| further) | glucuronide (pCG), indoxyl sulfate (IS), trimethylamine N- | | | |
| | oxide (TMAO), indole acetic acid (IAA), and | | | |
| | phenylacetylglutamine (PAG). | | | |
| | Secondary exploratory efficacy outcomes assesses change from | | | |
| | baseline in morning sample for urinary excretion of uremic | | | |
| | toxins (total and free p-cresyl sulfate (pCS), p-cresyl | | | |
| | glucuronide (pCG), indoxyl sulfate (IS), trimethylamine N- | | | |
| | oxide (TMAO), indole acetic acid (IAA), and | | | |
| | phenylacetylglutamine (PAG) and urine-albimun-creatinine | | | |
| | ratio (UACR). | | | |
| | Further exploratory clinical efficacy outcomes include | | | |
| | assessments of effects on: Body weight, waist circumference, | | | |
| | Bristol Stool Chart (BSC), IBS-C score, bowel movement | | | |
| | questionnaire, measures of appetite, fatigue score, the Dialysis | | | |
| | Symptom Index, QoL, Sleep quality, cognitive function, daily | | | |
| | activity, physical function, symptoms of skin itching, global | | | |
| | ureamia symptom score, blood pressure, and arterial stiffness. | | | |
| | Further exploratory analytical efficacy outcomes include | | | |
| | assessments of effects on: targeted metabolomics in feces and | | | |
| | plasma, changes in concentration of endotoxinemia (LPS), | | | |
| | change in serum markers of intestinal permeability (zonulin, d- | | | |
| | lactate) and gut trophic factors (epidermal growth factor (EGF) | | | |
| | and GLP-2), quality assessment of dialysis, changes in | | | |
| | nutritional status markers (albumin, pre-albumin), changes in | | | |
| | inflammatory markers (CRP, IL-6, TNF-alpha, IL-1b, IL-10). | | | |
| Efficacy Assessment | See previous text box | | | |
| Parameters (primary, | | | | |
| secondary, tertiary, | | | | |
| further) | | | | |
| Safety and | Adverse events, including gastrointestinal tolerability | | | |
| tolerability | (Gastrointestinal Symptom Rating Scale) and nausea, vital signs, | | | |
| Assessments | changes in blood biomarkers including effects on electrolytes (Na, K, | | | |
| | Ca, Mg, P, Cl, acid/base status) and renal function markers | | | |
| | (creatinine, urea, uric acid) | | | |
| Design | Description: | | | |
| | This is a prospective, randomized, double-blind, placebo-controlled, | | | |
| | multi-centre, multi-country, trial to study the effects of a nutritional | | | |
| | FSMP product (CmKD) containing synbiotics (e.g., a prebiotic [for | | | |
| | example, cellobiose] and a probiotic [for example, L johnsonii NCC | | | |
| | 533]) and gut/kidney energetic substrates (e.g., short and medium | | | |
| | chain triglycerides containing butyric (C4) and caprylic (C8) acid)) | | | |
| | on reducing uremic toxins in people with impaired kidney function. | | | |
| | Randomization/stratification: | | | |
| | Participants who successfully meet all the screening criteria are | | | |
| | randomized in a 2:1 ratio to either CmKD or placebo. | | | |
| | Duration: | | | |
| | The nutritional intervention (the FSMP or placebo) is provided for | | | |
| | approximately 12 weeks. Participants are followed for up to 13 weeks | | | |
| | including the screening period of up to 4 weeks. Each treatment visit | | | |
| | lasts approximately 1 hour. | | | |
| | A sample-size re-estimation/futility analysis (by an independent data | | | |
| | monitoring committee (DMC)) is carried out after 36 participants | | | |
| | have completed 12 weeks of treatment (e.g., 40% of the initial target | | | |
| | of 90 people). The purpose of this is two- fold: | | | |
| | 1) sample-size adjustment (an a-priori promising statistical zone is | | | |
| | defined to enable a potential re-estimation of study sample size to | | | |
| | demonstrate a meaningful effect size on uremic toxin reduction); | | | |
| | with a potential expansion of up to 150 participants, and | | | |
| | 2) futility assessment (in case no signal for likelihood to modulate | | | |
| | uremic toxin levels, the study is be terminated prematurely). | | | |
| | There is a separate DMC charter developed, and the DMC | | | |
| | recommendation is communicated to the steering committee. There | | | |
| | is no premature unblinding should the recommendation of continued | | | |
| | study conduct, with or without sample-size readjustment, be | | | |
| | provided. | | | |
| Trial population | The number of participants in the best case scenario are considered | | | |
| | necessary for randomization, are 105 participants to achieve | | | |
| | minimum 60 completers in the FSMP group and 30 in the placebo | | | |
| | group. In a revised scenario following potential sample-size | | | |
| | readjustment, 170 participants are considered necessary for | | | |
| | randomization to achieve 100 completers in the FSMP group and 50 | | | |
| | completers in placebo group. | | | |
| | Condition: Chronic kidney disease | | | |
| | Inclusion criteria: | | | |
| | Male or female 18-75 years | | | |
| | CKD stage 3b-5 with estimated glomerular filtration rate < 45 | | | |
| | ml/min/1.73 m2; including people on hemodialysis: ⅓ on | | | |
| | hemodialysis, ⅓ will have eGFR < 20 mL/min/1.73 m2, and | | | |
| | ⅓ will have eGFR 20-45 mL/min/1.73 m2 | | | |
| | No history of kidney transplantation | | | |
| | BMI 18-30 kg/m2 | | | |
| | Patient is followed at the recruiting nephrology departments | | | |
| | For women of childbearing age, at least one method of | | | |
| | contraception is recognized as effective | | | |
| | Adherence to recommended diet/protein content (according | | | |
| | to national/international standards followed) | | | |
| | Exclusion criteria: | | | |
| | Patients with progressive inflammatory, infectious, cardiovascular or | | | |
| | neoplastic disease (per investigator assessment) | | | |
| | Patients refusing a dietary follow-up | | | |
| | Patients having a planned transplant or new-onset dialysis in the next | | | |
| | 6 months. | | | |
| | Patients having a colectomy, resection of the small intestine or | | | |
| | cholecystectomy | | | |
| | Patients having received antibiotics ≥7 days in the last 3 months | | | |
| | Patients having received prebiotics, probiotics in the last 3 months. | | | |
| | Patients treated with more than 2 g of calcium per day | | | |
| | Patients using laxatives (more than 2 per day) | | | |
| | Patients having: Uncontrolled metabolic acidosis (bicarbonatemia < | | | |
| | 18 mM), hyperparathyroidism (PTH greater than 5 times the upper | | | |
| | limit of normal), hypercalcemia (Calcium > 2.55 mmol/L), | | | |
| | hypophosphoremia (<0.70 mmol/L), or anemia | | | |
| | (hemoglobinemia < 80 g/L) | | | |
| | Undernutrition: albumin < 38 g/L or prealbumin < 0.3 g/L | | | |
| | Known hypersensitivity to any of the substances or excipients of the | | | |
| | blend | | | |
| | Patient who is pregnant, breastfeeding or likely to become pregnant | | | |
| | during the study | | | |
| | Excessive amount of daily fibre intake (>4 servings per day of high- | | | |
| | fiber food assessed by “The habitual dietary fibre intake short food | | | |
| | frequency questionnaire” (DFI-FFQ)) | | | |
| Visits and flow | See also Flow chart below | | | |
| chart/visit schedules | There are 4 site visits in addition to screening (baseline (V1), week 3 | | | |
| | (V2), week 6 (V3), and week 12 (V4)) (table). Screening (Day −30 | | | |
| | to −1), Visit 1 (Day 1), Visit 2 (week 3 ± 3 days), Visit 3 (week 6 ± 3 | | | |
| | days), and Visit 4 (week 12 ± 3 days). | | | |
| | Telephone contact with site personnel is planned at week 9. | | | |
| | Study products to be provided to the participants at randomization | | | |
| | and at week 6. Compliance of product intake will be monitored. | | | |
| Treatment Groups: | Test product: CmKD is a specific synbiotic blend combined with a | | | |
| Intervention | gut/kidney energetic substrate (e.g. Cellobiose + L johnsonii | | | |
| Duration | NCC533 + C4/C8 + pea GOS) | | | |
| Regimen: | The test product is provided in a powder format | | | |
| Dosage | Reference control: A placebo matching to the test product is used as | | | |
| | control (canola oil and maltodextrin having the caloric equivalent) | | | |
| | The reference control is provided in powder format. | | | |
| | The daily dose (a total of X g) is split in two dosages daily and is | | | |
| | titrated by mixing the product, delivered in a sachet/stickpack, in the | | | |
| | desired cold drink (in minimum 100 mL of e.g., water, milk, orange | | | |
| | juice, soft-drinks) or in the desired cold semi-solid product (in | | | |
| | minimum 100 mL of e.g., yoghurt, cottage cheese, porridge, pudding, | | | |
| | soups), twice a day with a meal | | | |
| | The product is titrated as follows: | | | |
| | | Week 1 | Week 2 | Week 3+ |
| | Morning | ⅓ of full dose | ⅔ of full dose | Full dose (1 |
| | (e.g., | | | sachet/stickpack) |
| | breakfast) | | | |
| | Afternoon | ⅓ of full dose | ⅔ of full dose | Full dose (1 |
| | (e.g., | | | sachet/stickpack) |
| | lunch) | | | |
| | Total daily | X g | Y g | Z g |
| | dose of | | | |
| | CmKD/Placebo | | | |

| | |
|---|---|
| | One sachet/stickpack contains 10-15 g of CmKD |
| Dosing Regimen and | The investigational product (CmKD, or placebo) is consumed orally |
| mode of | twice daily in the morning and in the afternoon, together with a cold |
| administration | drink/semi-solid product (usually breakfast and dinner/supper). |
| | General advice for a suggested healthy diet for individuals with |
| | kidney disease, including recipes, is provided. |

All publications mentioned in the above specification are herein incorporated by reference. Various modifications and variations of the disclosed compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1: A method of lowering or avoiding accumulation of uremic toxins in cardiometabolic or neurodegenerative conditions, the method comprising administering a composition, wherein the composition comprises:
(a) a probiotic bacterium having at least one feature selected from the group consisting of
lacking a gene for producing at least one of urea, uric acid, p-cresol, p-cresyl sulfate, indoxyl sulfate, indole, indole acetic acid, indoxyl, trimethylamine, TMAO, dimethylglycine, betaine and/or glutarate;
lacking at least one bacterial enzyme selected from the group consisting of urease, carnitine monooxygenase, carnitine reductase, tryptophanase and hydroxyphenylacetate; and
expressing at least one bacterial enzyme selected from the group consisting of α-galactosidase, β-galactosidase, glucan 1,4-β-glucosidase, cellulase, β-fructofuranosidase and Licheninase;
(b) a prebiotic that is a carbohydrate or fiber, wherein the carbohydrate or fiber can be hydrolyzed by at least one bacterial enzyme selected from the group consisting of α-galactosidase, β-galactosidase, glucan 1,4-β-glucosidase, cellulase, β-fructofuranosidase and Licheninase; and
(c) a lipid selected from the group consisting of a triglyceride, a short chain fatty acid, a medium chain fatty acid and any mixture thereof, wherein the triglyceride comprises short chain fatty acids, medium chain fatty acids, or a mixture thereof.

2: The method according to claim 1, wherein the probiotic bacterium is selected from the group consisting of:
a. Bifidobacterium animalis subspecies lactis NCC 2818 (CNCM I-3446);
b. Bifidobacterium longum subspecies infantis NCC 341 (ATCC 15697 (T));
c. Bifidobacterium longum subspecies longum NCC 2705 (CNCM I-2618) (NCBI refseq; GCA_000007525.1);
d. Enterococcus faecium NCC 2768 (NCIMB 10415);
e. Lactobacillus johnsonii NCC 533 (CNCM I-1225) (NCBI refseq; GCA_000008065.1);
f. Lactococcus lactis NCC 2287 (CNCM I-4154);
g. Lacticaseibacillus paracasei NCC 2461 (CNCM I-2116);
h. Lacticaseibacillus rhamnosus NCC 4007 (CGMCC 1.3724);
i. Staphylococcus carnosus NCC 1052 (CNCM I-5400);
j. Staphylococcus carnosus NCC 971 (CNCM I-5398); and
k. Streptococcus thermophilus NCC 2496 (CNCM I-3915);
or the probiotic bacterium is a probiotic having a genome that has at least 95% Average Nucleotide Identity (ANI) to any one of the genomic sequences of the probiotic bacteria according to any one of a. to k. as defined above.

3: The method according to claim 1, wherein the probiotic bacterium is Lactobacillus johnsonii NCC 533 (CNCM I-1225) (NCBI refseq; GCA_000008065.1) or a probiotic bacterium having a genome that has at least 95% ANI to the genomic sequence of Lactobacillus johnsonii NCC 533 (CNCM I-1225) (NCBI refseq; GCA_000008065.1, SEQ ID NO: 1).

4: The method according to claim 1, wherein the prebiotic is selected from the group consisting of α-galacto-oligosaccharides (α-GOS), raffinose, β-galacto-oligosaccharides (β-GOS), and cello-oligosaccharides (COS), and a combination thereof.

5: The method according to claim 1, wherein the prebiotic is selected from the group consisting of soy GOS (α-GOS), pea GOS (α-GOS), bovine milk oligosaccharides (BMOS) (β-GOS), Vivinal GOS (β-GOS), Cellobiose, Cellotriose, Cellotetraose, soluble hydrolyzed wheat, human milk oligosaccharides (HMOs), soluble hydrolyzed oat and/or β-glucan hydrolysates containing at least one of cellobiose, cellotriose, or cellotetraose, and a combination thereof.

6: The method according to claim 1, wherein the lipid is selected from the group consisting of:
a. Triglycerides (TG) composed of a mixture of butyrate (C4:0) and medium chain fatty acids (MCFAs);
b. Triglycerides (TG) composed of a mixture of short and medium chain fatty acids (SMCFAs), preferably C4 and/or C8 fatty acids;
c. Triglycerides (TG) composed of a mixture of butyrate and long chain fatty acids (such as triglyceride composed of a mixture of butyrate and oleate);
d. Triglycerides composed of a medium chain fatty acid;
e. Triglycerides (TG) composed of a short chain fatty acid;
f. Short chain fatty acids that could be metabolized into ketone bodies; and
g. Short and medium chain fatty acids that could be metabolized into ketone bodies, preferably C4 and/or C8 fatty acids.

7: The method according to claim 2, wherein:
a. the probiotic bacterium is selected from the group consisting of the probiotic bacteria according to a. to k. as defined above, or the probiotic bacterium is a probiotic having a genome that has at least 90% ANI;
b. the prebiotic is selected from the group consisting of α-galacto-oligosaccharides/raffinose, β-galacto-oligosaccharides, cello-oligosaccharides, and a combination thereof;
c. the lipid is selected from the group consisting of a triglyceride, a short chain fatty acid, a medium chain fatty acid and any mixture thereof, wherein the triglyceride comprises butyrate, short chain fatty acids, medium chain fatty acids, or a mixture thereof,

8: The method according to claim 7, wherein the probiotic bacterium is Lactobacillus johnsonii NCC 533 (CNCM I-1225) (NCBI refseq; GCA_000008065.1) or a probiotic having a genome that has at least 90% ANI to the sequence of Lactobacillus johnsonii NCC 533 (CNCM I-1225) (NCBI refseq; GCA_000008065.1).

9: The method according to claim 7, wherein the prebiotic is selected from the group consisting of soy GOS (α-GOS), pea GOS (α-GOS), bovine milk oligosaccharides (BMOS) (β-GOS), Vivinal GOS (β-GOS), cellobiose, cellotriose, cellotetraose, soluble hydrolyzed wheat, soluble hydrolyzed oat and/or β-glucan hydrolysates containing at least one of cellobiose, cellotriose or cellotetraose, and a combination thereof.

10: The method according to claim 7, wherein the lipid is selected from the group consisting of:
a. Triglycerides (TG) composed of a mixture of butyrate and medium chain fatty acids (MCFAs);
b. Triglycerides (TG) composed of a mixture of short and medium chain fatty acids (SMCFAs);
c. Triglycerides (TG) composed of a mixture of butyrate and long chain fatty acids;
d. Triglycerides composed of a medium chain fatty acid;
e. Triglycerides (TG) composed of a short chain fatty acid;
f. Short chain fatty acids that could be metabolized into ketone bodies; and
g. Medium chain fatty acids that could be metabolized into ketone bodies.

11: The method according to claim 1, wherein the probiotic bacteria are contained in an amount between 10³ cfu to 10¹² cfu per daily dose.

12: The method according to claim 1, wherein the prebiotic is contained in an amount of 0.1 g to 30 g per daily dose.

13: The method according to claim 1, wherein the lipid is contained in an amount of 0.1 g to 30 g per daily dose.

14: The method according to claim 1, wherein the lipid is a triglyceride consisting of butyrate and caprylate or the lipid is a triglyceride consisting of butyrate and oleate.

15: The method according to claim 1, wherein the lipid is a triglyceride consisting of butyrate and caprylate.

16: The method according to claim 1 wherein the composition is selected from the group consisting of a food product, a nutritional composition, dietary supplement, a food for special medical purposes (FSMP), a nutritional supplement, a dairy-based drink, a low-volume liquid supplement, functional food product, functional beverage product, a meal replacement beverage, and combinations thereof.

17: The method according to claim 1, wherein the composition is selected from the group consisting of a powder, a tablet, a capsule, an oil formulation, an emulsion, an oil-in-water emulsion (o/w emulsion), a water-in oil emulsion (w/o emulsion) and combinations thereof.

18: The method according to claim 1, wherein the lowering or avoiding accumulation of uremic toxins in cardiometabolic or neurodegenerative conditions is effective for delaying progression of diseases and comorbidities and/or for managing symptoms and syndrome associated with toxic effects of uremic solutes of diseases and comorbidities.

19: The method according to claim 1, wherein the lowering or avoiding accumulation of uremic toxins in cardiometabolic or neurodegenerative conditions includes:
treatment or prevention of Kidney disease, including chronic and acute; dialysis and pre-dialysis; rare diseases, genetic- and metabolic-induced;
treatment or prevention of Uremic syndrome, including protein energy wasting, bone-loss, hyper anorexia, fatigue, or inflammation;
delay of advanced kidney disease complication, including anemia,
hyperphosphatemia, secondary hyperparathyroidism, bone disease, neurological conditions;
delay of kidney disease comorbidities, including cardiovascular disease;
prevention of the risk or management of malnutrition;
delay of the progression of cardiometabolic disease;
prevention of the risk or management of cardiovascular disease and comorbidities (diabetes); and/or
prevention of the risk or management of neurodegenerative and neurological conditions.

20: A kit of parts, suitable for use in lowering or avoiding accumulation of uremic toxins in cardiometabolic or neurodegenerative conditions, and comprising the composition as defined according to claim 1, comprising the probiotic, the prebiotic and the lipid in two or more separate containers.

21: A method of treatment of a cardiometabolic or neurodegenerative condition, the method comprising administering a composition to a patient suffering from an increase of uremic toxins in the cardiometabolic or neurodegenerative condition, wherein the composition comprises:
(a) a probiotic bacterium having at least one feature selected from the group consisting of
lacking a gene for producing at least one of urea, uric acid, p-cresol, p-cresyl sulfate, indoxyl sulfate, indole, indole acetic acid, indoxyl, trimethylamine, TMAO, dimethylglycine, betaine and/or glutarate;
lacking at least one bacterial enzyme selected from the group consisting of urease, carnitine monooxygenase, carnitine reductase, tryptophanase and hydroxyphenylacetate; and
expressing at least one bacterial enzyme selected from the group consisting of α-galactosidase, β-galactosidase, glucan 1,4-β-glucosidase, cellulase, β-fructofuranosidase and Licheninase;
(b) a prebiotic that is a carbohydrate or fiber wherein the carbohydrate or fiber can be hydrolyzed by at least one bacterial enzyme selected from the group consisting of α-galactosidase, β-galactosidase, glucan 1,4-β-glucosidase, cellulase, β-fructofuranosidase and Licheninase; and
a lipid selected from the group consisting of a triglyceride, a short chain fatty acid, a medium chain fatty acid and any mixture thereof, wherein the triglyceride comprises short chain fatty acids, medium chain fatty acids, or a mixture thereof.

22-36. (canceled)
